(19) United States
(12) Patent Application Publication
(10) Pub. No.: US 2025/0059290 A1
(43) Pub. Date: Feb. 20, 2025

(54) ANTI-OX40 ANTAGONISTIC ANTIBODIES FOR THE TREATMENT OF AUTOIMMUNE DISEASES

(71) Applicant: Ichnos Sciences SA, La Chaux-de-Fonds (CH)

(72) Inventors: Jonathan Back, La Chaux-de-Fonds (CH); Ernst Kriehuber, La Chaux-de-Fonds (CH)

(21) Appl. No.: 18/619,805

(22) Filed: Mar. 28, 2024

Related U.S. Application Data
(63) Continuation of application No. 17/056,168, filed on Nov. 17, 2020, now abandoned.

(30) Foreign Application Priority Data
May 18, 2018 (EP) 18173318.9

(51) Int. Cl. C07K 16/28; A61K 39/00; A61P 37/02

(52) U.S. Cl. CPC C07K 16/2878; A61P 37/02; A61K 2039/505

(57) ABSTRACT

The present invention relates to the use GBR830 for the treatment of OX40 mediated disorders and in particular to the modulation of Th1 and/or Th2 and/or Th17/Th22 markers.

### TECHNICAL FIELD

The present invention relates to the use of GBRS30 for the treatment of OX40 mediated disorders and in particular to the modulation of Th1 and/or Th2 and/or Th17/Th22 markers.

### BACKGROUND

OX40 (CD134), a co-stimulatory molecule of the tumor necrosis factor receptor/tumor necrosis factor superfamily (TNFRSF/TNFSF), is predominantly expressed on T-cells, including effector cells and Foxp3⁺ regulatory T-cells (Treg), 24 to 72 hours following activation. Its ligand, OX40L (CD252), is expressed on activated antigen presenting cells (APCs), including dendritic cells (DCs) and endothelial cells. OX40/OX40L engagement is key to potentiating T-cell responses triggered through the T-cell receptor (TCR), including: (I) expansion of effector T-cells and prolongation of their survival by suppressing apoptosis; (II) promoting and sustaining CD4+ T-cell memory; (III) facilitating adhesion and migration; and (IV) enhancing T-cell effector functions, such as cytokine production.

In autoimmunity, upregulation of OX40/OX40L plays a critical multi-functional role in disruption of T-cell tolerance by increasing cell survival and suppressing apoptosis, increasing cell proliferation, and amplifying cytokine production. The OX40/OX40L interaction bridges Th2 and Th1 pathways by inducing interferon gamma secretion and causing otherwise harmless autoreactive T-cells to acquire effector T-cell function. In atopic dermatitis (AD), one of the most common inflammatory skin disorders, for instance, OX40L+DCs are highly increased compared with psoriatic and normal skin, with greater expression of OX40 in AD lesions. Moreover, OX40 is usually upregulated at sites of inflammation, especially on infiltrating lymphocytes and on peripheral circulating lymphocytes. In human autoimmune diseases, OX40 and OX40L expression is consistently associated with inflamed tissues and often correlates with disease severity. Systemic lupus erythematous (SLE) is an example where the pathogenesis is believed to involve genetic factors, environmental triggers and immunological abnormalities of both innate and adaptive immunity including antibody responses. SLE patients display a clear infiltration of OX40L and OX40 expressing cells in affected skin or kidney biopsies.

Blockade of OX40-OX40L interaction using monocional antibody, represents a promising immunotherapy to inhibit disease-responsible effector and helper T-cell function.

### SUMMARY OF THE INVENTION

The present invention provides an anti-OX40 antagonist antibody, GBR830, for use in the treatment or prevention of OX40-mediated disorders. GBR830 (CAS Registry Number 2126777-87-3) is an investigational, first-in-class, humanized monoclonal IgG1 antibody specific for inhibiting OX40 to treat autoimmune and chronic inflammatory disorders.

The present invention relates to an anti-OX40 antagonist antibody for use in the treatment or prevention of DX40-mediated disorders, wherein said antagonist antibody induces modulation of Th1 and/or Th2 and/or Th17/Th22 markers.

Also provided by the present disclosure is a method for treating an OX40 mediated disorder by administering to a patient a therapeutically effective amount of the disclosed anti-OX40 antagonist antibody, wherein said anti-OX40 antagonist antibody induces modulation of Th1 and/or Th2 and/or Th17/Th22 markers.

According to one aspect of the present invention, the Th1 markers which are modulated by the disclosed anti-OX40 antagonist antibody are selected from the group comprising IFNγ and CXCL10.

According to another aspect of the present invention, the Th2 markers which are modulated by the disclosed anti-OX40 antagonist antibody are selected from the group comprising IL-31, CCL11, CCL17, and TSLPR.

According to a further aspect of the present invention, the Th17/Th22 markers which are modulated by the disclosed anti-OX40 antagonist antibody are selected from the group comprising IL-23p19, IL-8 and S100As.

In accordance with a certain aspect of the present invention, Th1 and/or Th2 and/or Th17/Th22 markers are downregulated.

In one embodiment, the present invention discloses an anti-OX40 antagonist antibody used for the treatment or prevention of an OX40-mediate disorder, wherein the OX40-mediated disorder is atopic dermatitis.

Also provided by the present disclosure is a method for treating an OX40 mediated disorder by administering to a patient therapeutically effective amount of the disclosed anti-OX40 antagonist antibody, wherein the OX40-mediated disorder is atopic dermatitis.

In a more particular embodiment of the present invention, the OX40-mediated disorder is moderate-to-severe atopic dermatitis.

In a preferred embodiment, the anti-OX40 antagonist antibody used for the treatment or prevention of atopic dermatitis, including moderate-to-severe atopic dermatitis, is administrated intravenously at two doses of about 10 mg/Kg of the patient body weight, around four weeks apart.

The present invention also provides a method for treating an OX40-mediated disorder, wherein the OX40-mediated disorder is atopic dermatitis, including moderate-to-severe atopic dermatitis, by intravenously administering to a patient the disclosed anti-OX40 antagonist antibody at two doses of about 10 mg/Kg of the patient body weight, around four weeks apart.

In accordance to another aspect of the present invention, the anti-OX40 antagonist antibody is used for the treatment or prevention of an OX40-mediated disorder selected from the group comprising rheumatoid arthritis, autoimmune uveitis, multiple sclerosis, lupus (such as systemic lupus erythematosus) and graft-versus-host disease (GVHD), scleroderma, hidradenitis, and ulcerative colitis.

The present invention also provides a method for treating an OX40-mediated disorder, wherein the OX40-mediated disorder is selected from the group comprising rheumatoid arthritis, autoimmune uveitis, multiple sclerosis, lupus (such as systemic lupus erythematosus) and graft-versus-host disease (GVHD), scleroderma, hidradenitis, and ulcerative colitis by administering to a therapeutically effective amount of the disclosed patient the disclosed anti-OX40antagonist antibody.

The term “human OX40” as used herein includes variants, isoforms, and species homologs of human OX40. Accordingly, antibodies of this disclosure may, in certain cases, cross-react with OX40 from species other than human. In certain embodiments, the antibodies may be completely specific for one or more human

OX40 proteins and may not exhibit species or other types of non-human cross-reactivity. The complete amino acid sequence of an exemplary human OX40 has Swiss-Prot accession number P43489. OX40 is also known as CD134, TNFRSF4, ACT35 or TXGP1 L. Human OX40 is designated GeneID: 7293 by Entrez Gene, and HGNC: 11918 by HGNC. OX40 has also been designated CD 134 (cluster of differentiation 134). OX40 can be encoded by the gene designated TNFRSF4/OX40.

The use of “human OX40” herein encompasses all known or as yet undiscovered alleles and polymorphic forms of human OX40. The terms “human OX40”, “OX40” or “OX40 Receptor” are used herein equivalently and mean “human OX40” if not otherwise specifically indicated.

The term “OX40 ligand” or “OX40L” are used herein equivalently and include OX40 ligand, specifically human OX40 ligand. OX40L is a member of the TNF superfamily and is also known as gp34 or CD252. OX40L has also been designated CD252 (cluster of differentiation 252) and has the sequence database accession number P23510 (Swiss-Prot) or Q6FGS4 (Uniprot). OX40L is expressed on the surface of activated B cells, T cells, dendritic cells and endothelial cells.

The term “antibody or fragment thereof that binds to human OX40” as used herein includes antibodies or a fragment thereof that binds to human OX40 e.g. human OX40 in isolated form, with an affinity (KD) of 500 nM or less, preferably 200 nM or less, more preferably 150 nM or less, more preferably 120 nM or less, even more preferably 1 10 nM or less. The term “antibody or fragment thereof that binds to human OX40” includes antibodies or antigenic binding fragments thereof.

The terms “antagonistic antibody” or “antagonist antibody” are used herein equivalently and include an antibody that is capable of inhibiting and/or neutralising the biological signalling activity of OX40, for example by blocking binding or substantially reducing binding of OX40 to OX40 ligand and thus inhibiting or reducing the signalisation pathway triggered by OX40 and/or inhibiting or reducing an OX40-mediated cell response like lymphocyte proliferation, cytokine expression, or lymphocyte survival. The term “antibody” as referred to herein includes whole antibodies and any antigen binding fragments or single chains thereof.

An “antibody” refers to a glycoprotein comprising at least two heavy (H) chains and two light (L) chains inter-connected by disulfide bonds, or an antigen binding fragment thereof. Each heavy chain is comprised of a heavy chain variable region (abbreviated herein as VH) and a heavy chain constant region. The heavy chain constant region is comprised of three domains, CHI, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR) with are hypervariable in sequence and/or involved in antigen recognition and/or usually form structurally defined loops, interspersed with regions that are more conserved, termed framework regions (FR or FW). Each VH and VL is composed of three CDRs and four FWs, arranged from amino-terminus to carboxy-terminus in the following order: FW1, CDR1, FW2, CDR2, FW3, CDR3, FW4. The amino acid sequences of FW1, FW2, FW3, and FW4 all together constitute the “non-CDR region” or “non-extended CDR region” of VH or VL as referred to herein.

The term “heavy chain variable framework region” as referred herein may comprise one or more (e.g., one, two, three and/or four) heavy chain framework region sequences (e.g., framework 1 (FW1), framework 2 (FW2), framework 3 (FW3) and/or framework 4 (FW4)). Preferably the heavy chain variable region framework comprises FW1, FW2 and/or FW3, more preferably FW1, FW2 and FW3. The term “light chain variable framework region” as referred herein may comprise one or more (e.g., one, two, three and/or four) light chain framework region sequences (e.g., framework 1 (FW1), framework 2 (FW2), framework 3 (FW3) and/or framework 4 (FW4)). Preferably the light chain variable region framework comprises FW1, FW2 and/or FW3, more preferably FW1, FW2 and FW3.

The variable regions of the heavy and light chains contain a binding domain that interacts with an antigen. The constant regions of the antibodies may mediate the binding of the immunoglobulin to host tissues or factors, including various cells of the immune system (e.g., effector cells) and the First component (Cl q) of the classical complement system.

Antibodies are grouped into classes, also referred to as isotypes, as determined genetically by the constant region. Human constant light chains are classified as kappa (CK) and lambda (CX) light chains. Heavy chains are classified as mu (μ), delta (δ), gamma (γ), alpha (α), or epsilon (ε), and define the antibody's isotype as IgM, IgD, IgG, IgA, and IgE, respectively. Thus, “isotype” as used herein is meant any of the classes and/or subclasses of immunoglobulins defined by the chemical and antigenic characteristics of their constant regions. The known human immunoglobulin isotypes are IgGI (IGHG1), IgG2 (IGHG2), IgG3 (IGHG3), IgG4 (IGHG4), IgAI (IGHAI), IgA2 (IGHA2), IgM (IGHM), IgD (IGHD), and IgE (IGHE). The so-called human immunoglobulin pseudo-gamma IGHGP gene represents an additional human immunoglobulin heavy constant region gene which has been sequenced but does not encode a protein due to an altered switch region (Bensmana M et al., (1988) Nucleic Acids Res. 16(7): 3108). In spite of having an altered switch region, the human immunoglobulin pseudo-gamma IGHGP gene has open reading frames for all heavy constant domains (CH1-CH3) and hinge. All open reading frames for its heavy constant domains encode protein domains which align well with all human immunoglobulin constant domains with the predicted structural features. This additional pseudo-gamma isotype is referred herein as IgGP or IGHGP. Other pseudo immunoglobulin genes have been reported such as the human immunoglobulin heavy constant domain epsilon PI and P2 pseudo-genes (IGHEP1 and IGHEP2). The IgG class is the most commonly used for therapeutic purposes. In humans this class comprises subclasses IgG1, IgG2, IgG3 and IgG4. In mice this class comprises subclasses IgG1, IgG2a, IgG2b, IgG2c and IgG3.

The present invention relates to an anti-OX40 antagonist antibody for use in the treatment of subjects suffering of an OX40-mediated disorders. Also provided by the present disclosure is a method for treating an OX40 mediated disorder by administering to a subject a therapeutically effective amount of the disclosed anti-OX40 antagonist antibody.

The present invention relates to an anti-OX40 antagonist antibody for use in the treatment of patients suffering of an OX40-mediated disorders. Also provided by the present disclosure is a method for treating an OX40 mediated disorder by administering to a patient a therapeutically effective amount of the disclosed anti-OX40 antagonist antibody.

As used herein, the term “subject” includes any human or nonhuman animal. The term “nonhuman animal” includes all vertebrates, e.g., mammals and non-mammals, such as nonhuman primates, sheep, dogs, cats, horses, cows, chickens, amphibians, reptiles, etc. Preferably the subject is human.

A “patient” for the purposes of the present invention includes both humans and other animals, preferably mammals and most preferably humans. Thus the antibodies of the present invention have both human therapy and veterinary applications. The term “treatment” or “treating” in the present invention is meant to include therapeutic treatment, as well as prophylactic, or suppressive measures for a disease or disorder. Thus, for example, successful administration of an antibody prior to onset of the disease results in treatment of the disease. As another example, successful administration of an antibody after clinical manifestation of the disease to combat the symptoms of the disease comprises treatment of the disease.

“Treatment” and “treating” also encompasses administration of an antibody after the appearance of the disease in order to eradicate the disease. Successful administration of an antibody after onset and after clinical symptoms have developed, with possible abatement of clinical symptoms and perhaps amelioration of the disease, comprises treatment of the disease. Those “in need of treatment” include mammals already having the disease or disorder, as well as those prone to having the disease or disorder, including those in which the disease or disorder is to be prevented.

The antibody or of the present invention can be administered via one or more routes of administration using one or more of a variety of methods known in the art. As will be appreciated by the skilled artisan, the route and/or mode of administration will vary depending upon the desired results. Preferred routes of administration include intravenous, intramuscular, intradermal, intraperitoneal, subcutaneous, spinal or other parenteral routes of administration, for example by injection or infusion. More preferred routes of administration are intravenous or subcutaneous. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, an antibody of the invention can be administered via a non-parenteral route, such as a topical, epidermal or mucosal route of administration, for example, intranasally, orally, vaginally, rectally, sublingually or topically. Preferably the anti-OX40 antagonist antibody is administered intravenously or subcutaneously.

In a certain embodiments, the anti-OX40 antagonist antibody is administrated intravenously at at least one dose of about 10 mg/Kg of the patient body weight. In a more specific embodiment, the anti-OX40 antagonist antibody is administrated intravenously at two doses of about 10 mg/Kg of the patient body weight, around four weeks apart.

Provided by the present invention is an anti-OX40 antagonist antibody for use in the treatment of OX40-mediated disorders, wherein the administration of said anti-OX40 antibody includes a loading dose on Day 1, followed by at least one maintenance dose.

The terms “maintenance dose” or “maintenance dosing” as used herein are interchangeable, and refer to a dose administered to a patient subsequently to a first dose, referred herein as loading 20 dose, one time or multiple times.

Also provided by the present disclosure is a method for treating an OX40 mediated disorder by administering to a patient a loading dose of said anti-OX40 antibody on Day 1, followed by at least one maintenance dose.

In a further embodiment of the present invention, the disclosed antibody is administered subcutaneously at loading dose comprised between about 50 mg and about 2 g on Day 1, followed by at least one maintenance dose comprised between about 20 mg and about 1 g, starting on a day comprised between Day 10 and Day 40.

In another embodiment, the antibody of the present invention is administered subcutaneously at a dose comprised between about 50 mg and about 2 g and/or at a dose comprised between about 20 mg and about 1 g.

According to one aspect of the present invention, the antibody of the present invention is administered subcutaneously the loading dose comprised between about 50 mg and about 2 g, or between about 100 mg and about 1.5 g, or between about 150 mg and about 1.2 g, or between about 150 mg and about 600 g. More specifically the loading dose is at least 50 mg, or at least 60 mg, or at least 70 mg, or at least 80 mg, or at least 90 mg, or at least 100 mg, or at least 150 mg, or at least 200 mg, or at least 250 mg, or at least 300 mg, or at least 350 mg, or at least 400 mg, or at least 450 mg, or at least 500 mg, or at least 550 mg, or at least 600 mg, or at least 650 mg, or at least 700 mg, or at least 750 mg, or at least 800 mg, or at least 850 mg, or at least 900 mg, or at least 950 mg, or at least 1 g, or at least 1.2 g, or at least 1.5 g. Even more specifically the loading dose is selected from the group comprising about 50 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1 g, 1.2 g, 1.5 g and 2g. The present invention also includes loading doses at any intermediate value between the above stated doses.

According to another aspect of the present invention, the maintenance dose is comprised between about 20 mg and about 1 g, or between about 50 mg and about 800 mg, or between about 70 mg and about 600 mg, or between about 70 mg and about 300 mg. More specifically the loading dose is at least 20 mg, or at least 30 mg, or at least 40 mg, or at least 50 mg, or at least 60 mg, or at least 70 mg, or at least 80 mg, or at least 90 mg, or at least 100 mg, or at least 150 mg, or at least 200 mg, or at least 250 mg, or at least 300 mg, or at least 350 mg, or at least 400 mg, or at least 450 mg, or at least 500 mg, or at least 550 mg, or at least 600 mg, or at least 700 mg, or at least 750 mg, or at least 800 mg, or at least 850 mg, or at least 900 mg, or at least 950 mg, or at least 1 g. Even more specifically the loading dose is selected from the group comprising about 20 mg, 30 mg, 40 mg, 50 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1 g. The present invention also includes loading and maintenance doses at intervals of 1, 5, and 10 mg between the above stated doses. The present invention also includes loading doses at any intermediate value between the above stated doses.

According to one aspect of the present invention, the loading dose is administered at Day 1.

According to another aspect of the present invention, the maintenance dose is administered starting on a day subsequent to Day 1. In certain embodiments, the maintenance dose is administered starting on a day comprised between about Day 2 and about Day 90. in a more preferred embodiment the maintenance dose Is administered starting on a day comprised between about Day 10 and about Day 40. In particular the maintenance dose is administered starting on a day selected from the group comprising Day 2, Day 8, Day 15, Day 22, Day 29, Day 36, Day 43, Day 50, Day 57, Day 64, Day 71, Day 78, Day 85, Day 92. In a specific embodiment the maintenance dose is administered starting on Day 15, or on Day 29. The present invention also includes that the maintenance dose is administered starting on 1, 2, 3, 4, 5, 6, 7, 8, 9, 10 day(s) subsequent to the above stated starting days.

According to another aspect of the present invention, the maintenance dose is administered every n days after the starting day, wherein n is equal to or greater than about 1 day and equal to or less than about 90 days. More preferably n is equal to or greater than about 10 day and equal to or less than about 40 days. In particular n is at least 1 day, at least 7 days, at least 14 days, at least 21 days, at least 28 days, at least 35 days, at least 42 days, at least 49 days, at least 56 days, at least 63 days, at least 70 days, at least 77 days, at least 84 days, at least 91 days. More specifically, n is selected from the group comprising 1 day, 7 days, 14 days, 21 days, 28 days, 35 days, 42 days, 49 days, 56 days, 63 days, 70 days, 77 days, 84 days, 91 days. In a preferred embodiment n is selected from the group comprising 15 days and 30 days. The present invention also includes that n at intervals of 1, 2, 3, 4, 5, 6, 7, 8, 9, 10 day(s) subsequent to the above stated n days.

In particular, the present invention provides an anti-OX40 antagonist antibody for use in the treatment of OX40-mediated disorders, wherein said antibody is administrated
(i) at loading dose equal to or greater than about 300 mg and equal to or less than about 1 g on Day 1, followed by at least one maintenance dose equal to or greater than about 100 mg and equal to or less than about 600 mg, starting on a day comprised between Day 10 and Day 20
(ii) at loading dose equal to or greater than about 300 mg and equal to or less than about 1 g on Day 1, followed by at least one maintenance dose equal to or greater than about 100 mg and equal to or less than about 600 mg, starting on a day comprised between Day 20 and Day 40
(iii) at loading dose equal to or greater than about 50 mg and equal to or less than about 300 mg on Day 1, followed by at least one maintenance dose equal to or greater than about 20 mg and equal to or less than about 150 mg, starting on a day comprised between Day 20 and Day 40
(iv) at loading dose equal to or greater than about 800 mg and equal to or less than about 1.5 g on Day 1, followed by at least one maintenance dose equal to or greater than about 300 mg and equal to or less than about 800 mg, starting on a day comprised between Day 10 and Day 20

In a particular aspect, the maintenance dose is administrated every n days after the loading dose, wherein n is: equal to or greater than 10 days and equal to or less than 20 days; or equal to or greater than 20 days and equal to or less than 40 days.

More specifically, the present invention provides an anti-OX40 antagonist antibody for use in the treatment of OX40-mediated disorders, wherein said antibody is administrated
(i) at loading dose of about 600 mg on Day 1, followed by at least one maintenance a dose of about 300 mg; or
(ii) at loading dose of about 600 mg on Day 1, followed by at least one maintenance a dose of about 300 mg; or
(iii) at loading dose of about 150 mg on Day 1, followed by at least one maintenance a dose of about 75 mg.
(iv) at loading dose of about 1.2 g on Day 1, followed by at least one maintenance a dose of about 600 mg.

Even more specifically, the present invention provides an anti-OX40 antagonist antibody for use in the treatment of OX40-mediated disorders, wherein said antibody is administrated
(i) at loading dose of about 600 mg on Day 1, followed by a maintenance dose of about 300 mg starting at Day 15 every 2 weeks; or
(ii) at loading dose of about 600 mg on Day 1, followed by a maintenance dose of about 300 mg starting at Day 29 every 4 weeks; or
(iii) at loading dose of about 150 mg on Day 1, followed by a maintenance dose of about 75 mg starting at Day 29 every 4 weeks.
(iv) at loading dose of about 1.2 g on Day 1, followed by a maintenance dose of about 600 mg starting at Day 15 every 2 weeks; or

Also provided by the present disclosure is a method for treating an OX40 mediated disorder by administering to a patient
(i) at loading dose equal to or greater than about 300 mg and equal to or less than about 1 g on Day 1, followed by at least one maintenance dose equal to or greater than about 100 mg and equal to or less than about 600 mg, starting on a day comprised between Day 10 and Day 20
(ii) at loading dose equal to or greater than about 300 mg and equal to or less than about 1 g on Day 1, followed by at least one maintenance dose equal to or greater than about 100 mg and equal to or less than about 600 mg, starting on a day comprised between Day 20 and Day 40
(iii) at loading dose equal to or greater than about 50 mg and equal to or less than about 300 mg on Day 1, followed by at least one maintenance dose equal to or greater than about 20 mg and equal to or less than about 150 mg, starting on a day comprised between Day 20 and Day 40
(iv) at loading dose equal to or greater than about 800 mg and equal to or less than about 1.5 g on Day 1, followed by at least one maintenance dose equal to or greater than about 300 mg and equal to or less than about 800 mg, starting on a day comprised between Day 10 and Day 20
   wherein the maintenance dose is administrated every n days after the loading dose, wherein n is equal to or greater than 10 days and equal to or less than 20 days; or equal to or greater than 20 days and equal to or less than 40 days.

More specifically, also provided by the present disclosure is a method for treating an OX40 mediated disorder by administering to a patient
(i) at loading dose of about 600 mg on Day 1, followed by at least one maintenance a dose of about 300 mg; or
(ii) at loading dose of about 600 mg on Day 1, followed by at least one maintenance a dose of about 300 mg; or
(iii) at loading dose of about 150 mg on Day 1, followed by at least one maintenance a dose of about 75 mg.
(iv) at loading dose of about 1.2 g on Day 1, followed by at least one maintenance a dose of about 600 mg;

Even more specifically, the present disclosure also provides a method for treating an OX40 mediated disorder by administering to a patient
(i) at loading dose of about 600 mg on Day 1, followed by a maintenance dose of about 300 mg starting at Day 15 every 2 weeks; or
(ii) at loading dose of about 600 mg on Day 1, followed by a maintenance dose of about 300 mg starting at Day 29 every 4 weeks; or
(iii) at loading dose of about 150 mg on Day 1, followed by a maintenance dose of about 75 mg starting at Day 29 every 4 weeks.
(iv) at loading dose of about 1.2 g on Day 1, followed by a maintenance dose of about 600 mg starting at Day 15 every 2 weeks.

As used herein, the term “OX40-mediated disorder” includes conditions such as allergy, asthma, COPD, rheumatoid arthritis, psoriasis and diseases associated with autoimmunity and inflammation. In particular, according to the present invention, exemplary OX40 mediated disorders include infections (viral, bacterial, fungal and parasitic), endotoxic shock associated with infection, arthritis, rheumatoid arthritis, asthma, chronic obstructive pulmonary disease (COPD), pelvic inflammatory disease, Alzheimer's Disease, inflammatory bowel disease, Crohn's disease, ulcerative colitis, Peyronie's Disease, coeliac disease, gallbladder disease, Pilonidal disease, peritonitis, psoriasis, vasculitis, surgical adhesions, stroke, Type I Diabetes, lyme disease, arthritis, meningoencephalitis, autoimmune uveitis, immune mediated inflammatory disorders of the central and peripheral nervous system such as multiple sclerosis, lupus (such as systemic lupus erythematosus) and Guillain-Barr syndrome, Atopic dermatitis, autoimmune hepatitis, fibrosing alveolitis, Grave's disease, IgA nephropathy, idiopathic thrombocytopenia purpura, Meniere's disease, pemphigus, primary biliary cirrhosis, sarcoidosis, scleroderma, Wegener's granulomatosis, pancreatitis, trauma (surgery), graft-versus-host disease (GVHD), transplant rejection, cardiovascular disease including ischaemic diseases such as myocardial infarction as well as atherosclerosis, intravascular coagulation, bone resorption, osteoporosis, osteoarthritis, periodontitis, hypochlorhydia and neuromyelitis optica, hidradenitis.

Other exemplary OX40 mediated disorder include infections (viral, bacterial, fungal and parasitic), endotoxic shock associated with infection, arthritis, rheumatoid arthritis, asthma, bronchitis, influenza, respiratory syncytial virus, pneumonia, chronic obstructive pulmonary disease (COPD), idiopathic pulmonary fibrosis (IPF), cryptogenic fibrosing alveolitis (CFA), idiopathic fibrosing interstitial pneumonia, emphysema, pelvic inflammatory disease, Alzheimer's Disease, inflammatory bowel disease, Crohn's disease, ulcerative colitis, Peyronie's Disease, coeliac disease, gallbladder disease, Pilonidal disease, peritonitis, psoriasis, vasculitis, surgical adhesions, stroke, Type I Diabetes, lyme disease, arthritis, meningoencephalitis, autoimmune uveitis, immune mediated inflammatory disorders of the central and peripheral nervous system such as multiple sclerosis, lupus (such as systemic lupus erythematosus) and Guillain-Barr syndrome, Atopic dermatitis, autoimmune hepatitis, fibrosing alveolitis, Grave's disease, IgA nephropathy, idiopathic thrombocytopenia purpura, Meniere's disease, pemphigus, primary biliary cirrhosis, sarcoidosis, scleroderma, Wegener's granulomatosis, pancreatitis, trauma (surgery), graft-versus-host disease (GVHD), transplant rejection, cardiovascular disease including ischaemic diseases such as myocardial infarction as well as atherosclerosis, intravascular coagulation, bone resorption, osteoporosis, osteoarthritis, periodontitis, hypochlorhydia and neuromyelitis optica, hidradenitis.

In accordance to a preferred aspect of the present invention, the anti-OX40 antagonist antibody is used for the treatment or prevention of an OX40-mediated disorder selected from the group comprising atopic dermatitis, rheumatoid arthritis, autoimmune uveitis, multiple sclerosis, lupus (such as systemic lupus erythematosus), ulcerative colitis, scleroderma, hidradenitis and graft-versus-host disease (GVHD).

In one embodiment of the present invention, the anti-OX40 antagonist antibody is GBR 830 (CAS Registry Number 2126777-87-3).

In a more specific embodiment of the present invention, the OX40-mediate disorder is atopic dermatitis, wherein atopic dermatitis is mild, or mild-to-moderate, or moderate, or moderate-to-severe, or severe. In an even more specific embodiment, OX40-mediate disorder is moderate-to-severe atopic dermatitis.

Atopic dermatitis, Atopic dermatitis” (AD), as used herein, means an inflammatory skin disease characterized by intense pruritus (e.g., severe itch) and by scaly and dry eczematous lesions. The term “atopic dermatitis” includes, but is not limited to, AD caused by or associated with epidermal barrier dysfunction, allergy (e.g., allergy to certain foods, pollen, mold, dust mite, animals, etc.), radiation exposure, and/or asthma. The present invention encompasses methods to treat patients with mild, moderate-to-severe or severe AD. As used herein, “moderate-to-severe AD”, is characterized by intensely pruritic, widespread skin lesions that are often complicated by persistent bacterial, viral or fungal infections, Moderate-to-severe AD also includes chronic AD in patients. In many cases, the chronic lesions include thickened plaques of skin, lichenification and fibrous papules. Patients affected by moderate-to-severe AD also, in general, have more than 10% of the body's skin affected, or 10% of skin area in addition to involvement of the eyes, hands and body folds. Moderate-to-severe AD is also considered to be present in patients who require frequent treatment with topical corticosteroids. A patient may also be said to have moderate-to-severe AD when the patient is resistant or refractory to treatment by either a topical corticosteroid or a calcineurin inhibitor or any other commonly used therapeutic agent known in the art.

The present invention provides materials and methods for improving one or more Atopic dermatitis efficacy parameter(s) in a subject. Examples of “AD related efficacy parameters” include: (a) Scoring of Atopic Dermatitis-SCORAD (b) Investigators Global Assessment (IGA); (c) Pruritus Numerical rating scale (NRS) (d) Dermatology Life Quality Index-DLQI (e) Body Surface Area (BSA); (f) Eczema Area and Severity Index (EASI); (h) and trans-epidermal water loss (TEWL). An “improvement in an AD related efficacy parameters” means a decrease from baseline of one or more of IGA, BSA, EASI, SCORAD, TEWL, DLQI or NRS. As used herein, the term “baseline,” with regard to an AD-related efficacy parameters, means the numerical value of the AD-related efficacy parameters for a subject prior to or at the time of administration of a pharmaceutical composition of the present invention SCORAD. The SCORAD is a validated tool used in clinical research and clinical practice that was developed to standardize the evaluation of the extent and severity of AD (Dermatology 1993). The extent of AD is assessed as a percentage of each defined body area and reported as the sum of all areas, with a maximum score of 100% (assigned as “A” in the overall SCORAD calculation). The severity of 6 specific symptoms of AD is assessed using the following scale: none (0), mild (1), moderate (2), or severe (3) (for a maximum of 18 total points, assigned as “B” in the overall SCORAD calculation). Subjective assessment of itch and sleeplessness is recorded for each symptom by the patient or relative on a visual analogue scale (VAS), where 0 is no itch (or sleeplessness) and 10 is the worst imaginable itch (or sleeplessness), with a maximum possible score of 20. This parameter is assigned as “C” in the overall SCORAD calculation. The SCORAD is calculated as: A 5+7B/2+C (Kunz et al, 1997). Investigators Global Assessment (IGA). The IGA is an assessment scale used in clinical studies to determine severity of AD and clinical response to treatment based on a 5-point scale ranging from 0 (clear) to 4 (severe/very severe).

Pruritus Numerical rating scale (NRS): The Pruritus NRS is a single-question assessment tool that is used to assess a subject's worst itch, on a scale of 1 to 10, as a result of AD in the previous 12 hours. Patients will record once daily and respond to the following question, “On a scale of 0-10, with 0 being no itch and 10 being the worst itch imaginable, how would you rate your worst degree of itch during the previous 24 hours?” Patient compliance on the pruritus NRS will be followed at each clinic visit.

Dermatology Life Quality Index (DLQI): The DLQI is a simple, patient-administered, 10-question, validated, quality-of-life questionnaire that covers 6 domains including symptoms and feelings, daily activities, leisure, work and school, personal relationships, and treatment. Response categories include “a little,” “a lot,” and “very much” with corresponding scores of 1, 2, and 3 respectively and “not at all”, “not relevant” responses scored as “0.” Totals range from 0 to 30 (less to more impairment) and a 5-point change from baseline is considered clinically relevant (Basra et al, 2008; Finlay et al, 1994).

Body Surface Area (BSA) BSA is assessed for each major section of the body (head, trunk, arms and legs) and is reported as a percentage of all major body sections combined.

Eczema Area and Severity Index (EASI), The EASI is a validated measure used in clinical practice and clinical trials to assess the severity and extent of AD. Four AD disease characteristics will be assessed for severity by the investigator or designee on a scale of “0” (absent) through “3” (severe). In addition, the area of AD involvement will be assessed as a percentage by body area of head, trunk, arms, and legs and converted to a score of 0 to 6 (Hanifin, 2001).

The present invention also includes methods involving the use, quantification, and analysis of Atopic dermatitis biomarker parameters. As used herein, the term “Atopic dermatitis biomarker parameters” means any biological response, cell type, parameter, protein, polypeptide, enzyme, enzyme activity, metabolite, nucleic acid, carbohydrate, or other biomolecule which is present or detectable in an AD patient at a level or amount that is different from (e.g., greater than or less than) the level or amount of the marker present or detectable in a non-AD patient. In some embodiments, the term “Atopic dermatitis biomarker parameters” includes a biomarker associated with Type 2 helper T-cell Th2)-driven inflammation. In order to evaluate for the drug effect or how much of the disease profile has been reversed by treatment as measured changes in the AD transcriptome using gene arrays consisting of differentially expressed genes between lesional and non lesional AD skin as defined by fold changes (typically a fold change of more than 2).

The AD disease phenotype is the integration of cellular and molecular markers that define the epidermal pathology (hyperplasia, differentiation abnormalities), and Th2, and Th22 immune activation. The changes or reversal of these immune and barrier defects is assessed by IHC and RT-PCR.

Other exemplary AD-associated biomarkers include a panel of Th1, Th2, Th17, Th22 cytokines, chemokines and related protein that are shown as elevated in AD blood and to decrease with treatment. Exemplary AD-associated biomarkers include but are not limited to, e.g., MMP12, IL17A, IL22, IL23p40, IL13, IL5, IFNγ, CXCL10, IL-31, CCL11, CCL17, CCL18, CCL26, OX40L, TSLPR, FOXP3, IL-23p19, IL-18, S100As, Serum Thymus and activation-regulated chemokine (TARC/CCL17), eotaxin-3, total 5 Immunoglobulin E (IgE), Thymus and activation-regulated chemokine is a chemokine, shown to be strongly associated with disease severity in AD, and may be involved in pathogenesis of the disease. Baseline TARC levels will be assessed for potential predictive value for treatment response. Eotaxin-3 (CCL26), Eotaxin-3 is a chemokine, shown to be associated with disease severity in AD, and may be involved in pathogenesis of the disease. Baseline eotaxin-3 levels will be assessed for potential 10 predictive value for treatment response. Post-treatment samples will be evaluated for effects of anti OX40 antagonist antibody on eotaxin-3. Total Immunoglobulin E (IgE), Patients with AD often have elevated IgE. Total IgE levels have been found to modestly correlate with AD severity and may be involved in the pathogenesis of the disease. Changes in total IgE reflects not only on AD, but atopy in general. Baseline IgE levels will be assessed for potential predictive value for treatment response. 15 Trans-epidermal water loss (TEWL). Transepidermal water loss is a skin barrier function test that measures perspiration or water loss through the skin. This procedure involves the non-invasive application of a probe on the surface of the skin on the arm or leg. Affected and non-affected areas of skin will be tested.

Specifically with respect to SLE, the pathogenesis of SLE is believed to involve genetic factors, environmental triggers and immunological abnormalities of both innate and adaptive immunity including antibody responses. T helper cells are central players orchestrating the Interplay between innate antigen presenting cells and autoimmune B cell responses and a high dependency on co-stimulation pathways to provide essential signals for the initiation, perpetuation and eventually attenuation of inflammatory responses. The relative contribution of these multiple co-stimulation pathways to autoimmune pathologies such as SLE is only partially understood. OX40 is predominantly expressed on activated T-cells, including effector cells and Foxp3+ regulatory T-cells (Tregs). OX40L is expressed on activated APCs including dendritic cells, monocytes, B lymphocytes and endothelial cells.

OX40/0X40L engagement potentiates T-cell responses by the: expansion of effector T cells and leading to a prolongation in their survival; enhancing T-cell effector functions, such as cytokine production; promoting CD4+ T cell memory formation and reactivation; facilitating adhesion and migration through inflamed endothelium; promoting conversion of regulatory T cells (Tregs) into non-suppressive cells.

Genetic evidence has highlighted that OX40L is a risk factor for human immune-mediated disorders. Single nucleotide polymorphisms (SNP) in the OX40L/TNFSF4 locus are found tightly associated with SLE in many studies and further genetic analyses support also a link of TNFSF4 with pSS and SSc.

While genetic evidence establishes a causal link between OX40L and human disease, and concomitant expression of OX40/OX40L in SLE points at pathway engagement, the relative contribution and requirement to the pathogenesis of e.g. SLE is not well understood.

In human autoimmune diseases, OX40 and OX40L expression is consistently associated with inflamed tissues and often correlates with disease severity. SLE patients display a clear infiltration of OX40L and OX40 expressing cells in affected skin or kidney biopsies. OX40L expression on myeloid cells and OX40 expression on T helper cells in peripheral blood correlate with SLEDAI score, suggesting that OX40 pathway engagement is potentially active in lupus. Recent literature investigates the role of OX40 in human SLE pathogenesis and points at OX40 dependent alteration of Tfh and Treg responses (Jacquemin 2015; Richez 2018).

These studies highlight in particular a potential perpetuation loop orchestrated by immune complexes-dependent OX40L induction on myeloid APCs, promoting naïve and memory T helper cells to adopt a Tfh phenotype, in turn supporting enhanced B cell activity. In addition, APCs from active SLE patients mediated Treg dysfunction in an OX40L-dependent manner.

It remains to be determined whether OX40 is important for SLE induction or chronicity and hence whether interventions blocking OX40/OX40L interactions can be efficacious in immune mediated disease such as SLE.

Dose regimen for SLE is based upon all available safety and PK data from clinical studies along with the in vitro receptor occupancy data of GBR 830 in human whole blood.

GBR 830 is safe and well tolerated up to 40 mg/kg SD and up to 20 mg/kg q1 week repeat dosing.

The receptor occupancy experiments suggest that around 25 μg/ml of GBR 830 will lead to maximum receptor occupancy (ROmax).

In a preferred embodiment a dosage regimen of 600 mg LD followed by 300 mg q2week is expected to give a mean Ctrough of ˜46 μg/mL. Majority of subjects will have Ctrough ≥25 μg/mL. Therefore this regimen was considered for the treatment of SLE.

Atopic dermatitis is considered a polar Th2 disease. Chronic AD lesions have been shown to have a marked increase in Th2 T cells and related cytokines. OX40 mediates signaling by thymic stromal lymphopoietin (TSLP)-activated dendritic cells (DCs) and is highly upregulated in atopic skin. TSLP-activated DCs have been shown to preferentially activate Th2 T-cell responses in autologous and allogeneic cultures in an OX40-dependent manner. Therefore, GBR 830 may hold the promise for a more targeted, effective and less toxic approach to systemic therapy in AD. Preclinical pharmacology studies demonstrated that GBR 830 is able to block the interaction between OX40 and OX40L and suppress T cell proliferation and allogeneic reactions, such as mixed lymphocyte reactions, with 50% effective concentrations ranging from 0.1 to 3 μg/ml. These studies also demonstrated that GBR 830 has antibody-dependent cellular cytotoxicity and complement-dependent cytotoxicity potential.

Secondary pharmacodynamics studies were conducted to support the safety of GBR 830 administration in humans. GBR 830 was devoid of agonistic potential and did not induce cytokine release in either human peripheral whole blood from healthy subjects or human peripheral blood mononuclear cell cultures at high density. Taken together, these studies suggest a low risk of inadvertent cytokine release in humans for GBR 830.

In vitro vaccine reactivation assays suggest that targeting pathological responses driven by memory T cell reactivation with GBR 830 is relevant and potentially more efficacious than blocking CD28 signals. In vivo studies, done to evaluate the effect of GBR 830 treatment on a T dependent antibody response to keyhole limpet hemocyanin in cynomolgus monkey, suggest that targeting OX40 has more profound effects on late and memory responses than primary responses. This feature highlights that an antagonistic OX40 treatment in the clinic may display a safer profile in terms of infection susceptibility compared with broader immunosuppressors.

Consistent with in vitro data, in vivo pharmacology studies demonstrated that GBR 830 could suppress a xenogeneic reaction in a human-mouse GvHD model (mainly prophylactic) at doses as low as 1 mg/kg. Studies using a human psoriatic skin transplant model demonstrated the potent therapeutic anti-psoriatic activity of GBR 830 at doses as low as 1 mg/kg. The efficacy in these studies was on par with or better than established drugs (efalizumab, etanercept, clobetasol propionate, cyclosporin). These data confirm the immunomodulatory capabilities of GBR 830 in T cell mediated autoimmune and inflammatory conditions.

GBR 830 was well tolerated without any toxicologically significant, treatment related adverse findings in repeat dose toxicity studies of 6 weeks and 26 weeks duration in cynomolgus monkeys up to the dose levels of 100 mg/kg/week intravenous (IV) and 100 mg/kg/week subcutaneously (SC). The no observed adverse effect level (NOAEL) was 100 mg/kg/week after IV or SC administration for 26 weeks.

No visible reactions or adverse histopathologic changes at the IV and SC injection sites were noted after repeated administrations in the 6-week or 26-week toxicity studies in monkeys or single SC injection in rabbits, indicating no issues with local tolerability of GBR 830.

Additionally, GBR 830 has been investigated after both IV and SC administration and included 122 healthy volunteers and 62 subjects with AD (GBR 830-201). GBR 830 was safe and well tolerated in healthy volunteers up to 10 mg/kg IV after single dose administration.

In healthy volunteers, 2 phase 1 studies have been completed: GBR 830-101, a single ascending dose study, and GBR 830-102, an absolute bioavailability study. Study conduct for a phase 2a study in subjects with moderate-to-severe AD (GBR 830-201) has also been completed. A phase 1 single and multiple ascending dose study in healthy adult volunteers (GBR 830-103) is ongoing.

In the first-in-human phase 1 study GBR 830-101, the safety, tolerability, PK and immunogenicity of GBR 830 were evaluated following a single IV infusion over a dose range of 0.3 mg/kg to 10 mg/kg. GBR 830 was safe and well tolerated across the tested dose range. The serum exposures (maximum observed serum concentration [Cₘₐₓ] and area under the curve (AUC) from time 0 to infinity [AUC_{0-∞}) increased in a dose proportional manner. Six out of 34 subjects who received GBR 830 showed a positive anti-drug antibody (ADA) response, of whom 2 subjects had neutralizing antibodies.

In study GBR 830-102, the pharmacokinetics, immunogenicity, safety and tolerability were evaluated following a single SC injection of GBR 830 at 75 mg and 600 mg, and following a single IV infusion of GBR 830 at 600 mg. After SC injection, GBR 830 showed an average absolute bioavailability of approximately 65%. GBR 830 concentrations in serum increased gradually; median time at which Cₘₐₓ is observed (tₘₐₓ) was approximately 4 to 5 days. When compared at the same dose level, the Cₘₐₓ after SC injection was approximately 3.2-fold lower than the IV infusion. A lower incidence of ADA was observed with higher doses (600 mg SC: 1 out of 15 subjects; 600 mg IV: 1 out of 10 subjects) compared to the lower dose (75 mg SC: 10 out of 15 subjects). GBR 830 was well-tolerated after IV and SC dosing, and fixed dosing by the SC route was determined to be an acceptable path forward.

In the phase 2a study (GBR 830-201), the safety, biological activity, pharmacokinetics and immunogenicity were evaluated in subjects with moderate-to-severe AD, following 2 consecutive IV infusions of GBR 830 (10 mg/kg) administered approximately 4 weeks apart. GBR 830 was found to be safe and well tolerated. With 2 doses, given 4 weeks apart, GBR 830 showed minimal accumulation (1.16 to 1.22-fold) in Cₘₐₓ, AUC over dosing interval (AUC₀₋ₜₐᵤ), and serum concentration at end of dosing interval (C_{trough}). Anti-drug antibodies were detected in 6 out of 46 subjects.

Preliminary analysis of GBR 830-201 safety data showed that in AD patients, GBR 830 was safe and well tolerated after 2 repeated dose administrations of 10 mg/kg IV 4 weeks apart. Out of 62 subjects, 39 (63%) experienced at least 1 treatment-emergent adverse event (TEAE); there was an equal proportion of subjects with at least 1 TEAE in both the GBR 830 and placebo treatment groups. In this study, 1 GBR 830 patient experienced a serious adverse event (SAE) (coronary artery occlusion: left anterior descending coronary artery blockage was due to pre-existing cardiovascular disease), which was assessed by the Investigator as not related to study drug.

Preliminary analysis of clinical efficacy of the non-powered, randomized, placebo-controlled GBR 830-201 study suggests positive results in GBR 830-treated subjects compared to baseline. Starting on Day 15 after the first 2 infusions, GBR 830-treated subjects showed a persistent and increased improvement le, lowering in mean SCORing Atopic Dermatitis (SCORAD) and Eczema Area and Severity Index EASI clinical scoring vs placebo. Over the duration of the study, a mean reduction in EASI scoring was noted with GBR 830 treatment compared to placebo on Days 57 and 71. Persistent improvement in clinical outcome with GBR 830 treatment can be tracked through EASI scoring from the end of the treatment period to the end of study follow-up. Clinical improvement was associated with a decline in messenger RNA (mRNA) biomarkers for disease activity, including Th1 and Th22 pathways, indicating an effect on both acute and chronic stages of AD. Overall, across the studies GBR 830 showed favorable linear pharmacokinetic (PK) profile with slow clearance and long elimination half-life (t½ approximately 10 to 15 days) after IV infusion and SC injection.

### EXAMPLES

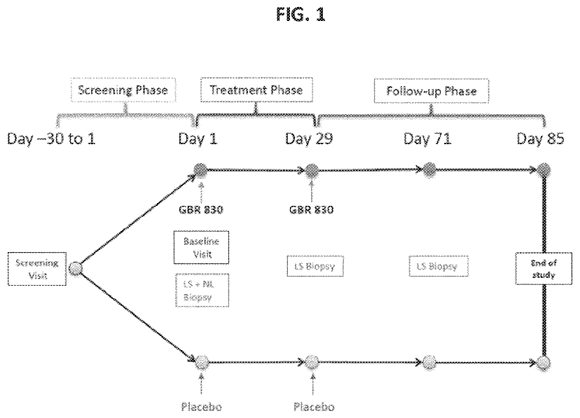

FIG. 1. Study design

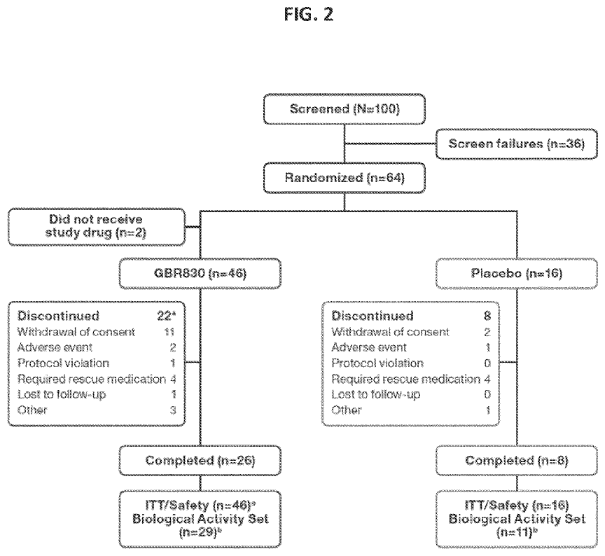

FIG. 2 Subject disposition. * Includes subjects who did not receive GBR830 (n=2). ^{a} Excludes subjects from the randomized population (GBR830, n=2) who did not receive ≥1 partial or full dose of study treatment. ^{b} Excludes subjects from the ITT population (GBR830, n=17; placebo, n=5) who did not receive both doses of study drug and have 21 post-baseline skin biopsy (Day 29 or Day 71) ITT, intent-to-treat.

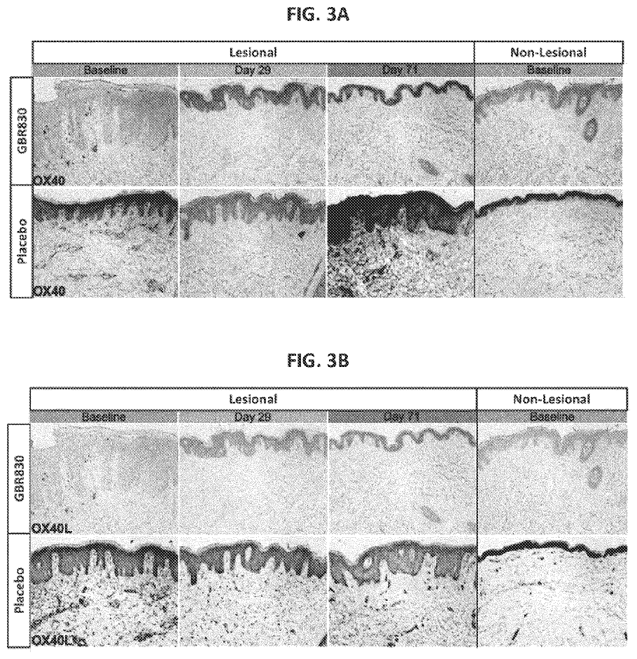

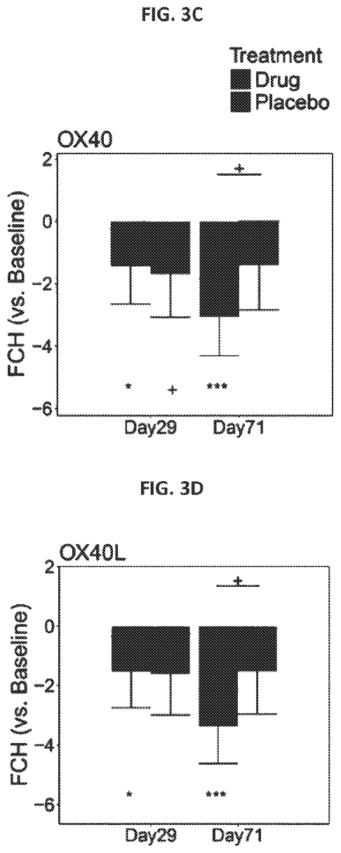

FIG. 3. Immunohistochemistry images of OX40 target from representative GBR830-and placebo-treated subjects Immunohistochemistry staining of OX40 (A) and OX40L (B) at baseline and after treatment (Day 29 and Day 71); protein expression is shown in red staining and the images in each line were taken from the same subject. Mean fold change (FCH) from baseline in OX40 expression (C) and OX40L expression (D). +P<0.1, *P<0.05, **P<0.01, ***P<0.001.

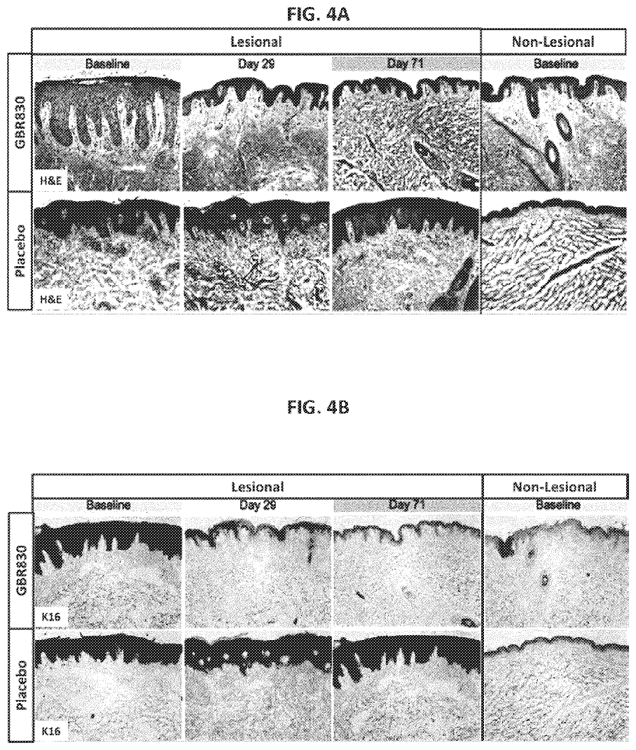

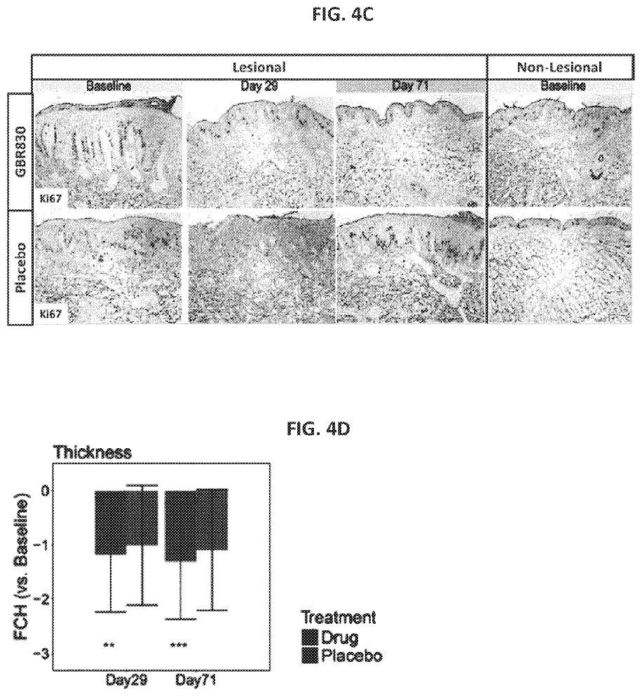

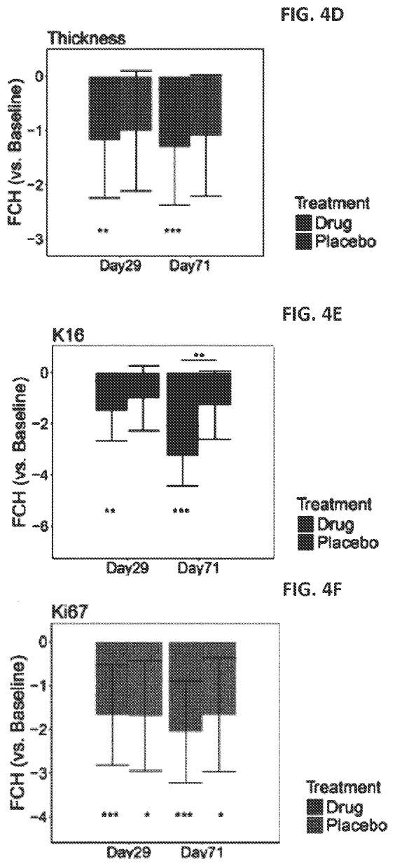

FIG. 4. Immunohistochemistry images for representative drug and placebo subjects, at baseline and after treatment (A-C), and quantification of epidermal proliferation markers (D-F). Panels show (A) H&E staining, (B) K16 staining, and (C) Ki67 staining showing epidermal hyperplasia at baseline lesions; the images for both drug and placebo panels were taken from a representative subject for each. Mean fold change (FCH) from baseline is shown for (D) epidermal thickness, (E) K16 mRNA expression measured by RT-PCR, and (F) Ki67 protein expression measured by immunohistochemistry. +P<0.1, *P<0.05, **P<0.01, ***P<0.001. H&E, hematoxylin and eosin; K16, keratin 16; RT-PCR, real-time polymerase chain reaction.

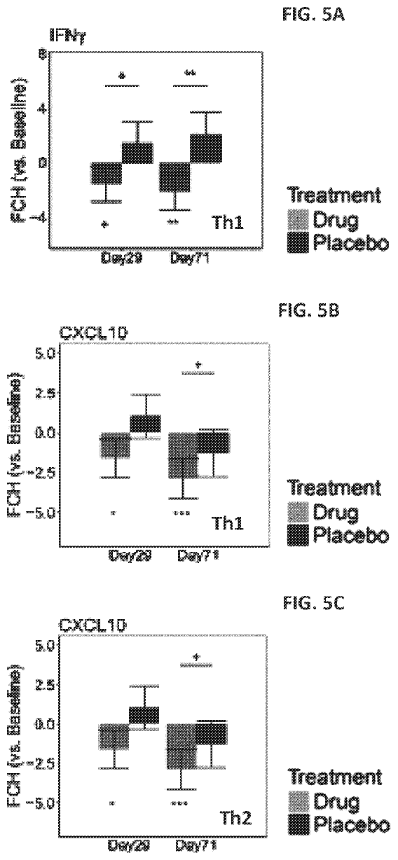

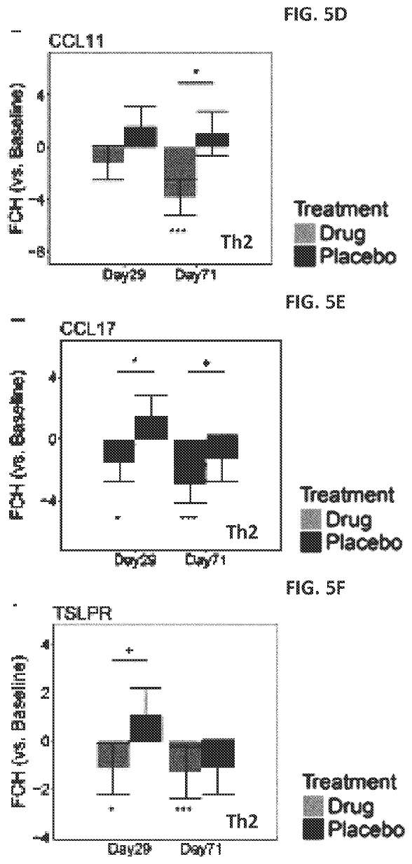

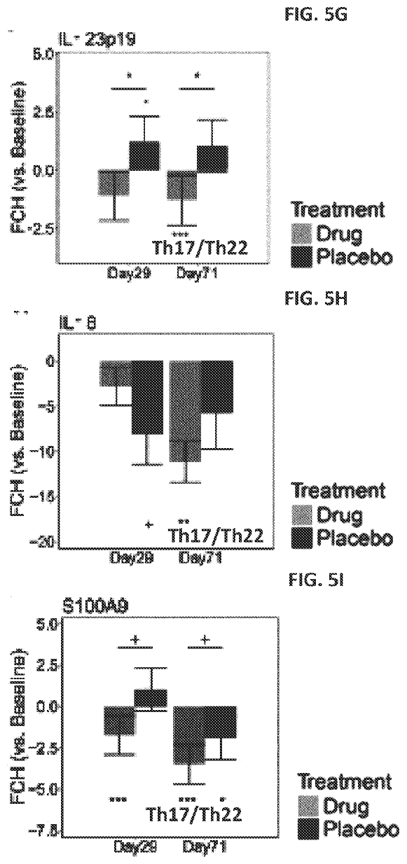

FIG. 5. Changes in quantitative RT-PCR mRNA expressions following treatment. Significant reductions in mRNA expressions of representative inflammatory markers of Th1 (A-B), Th2 (C-E), and Th17/Th22 (F-H) pathways were observed in subjects treated with GBR830, as compared to baseline and also to placebo. +P<0.1, *P<0.05, **P<0.01, ***P<0.001. mRNA, messenger ribonucleic acid; RT-PCR, real-time polymerase chain reaction.

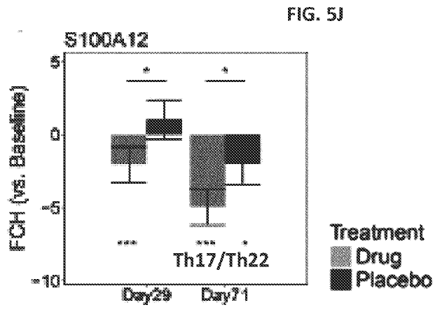

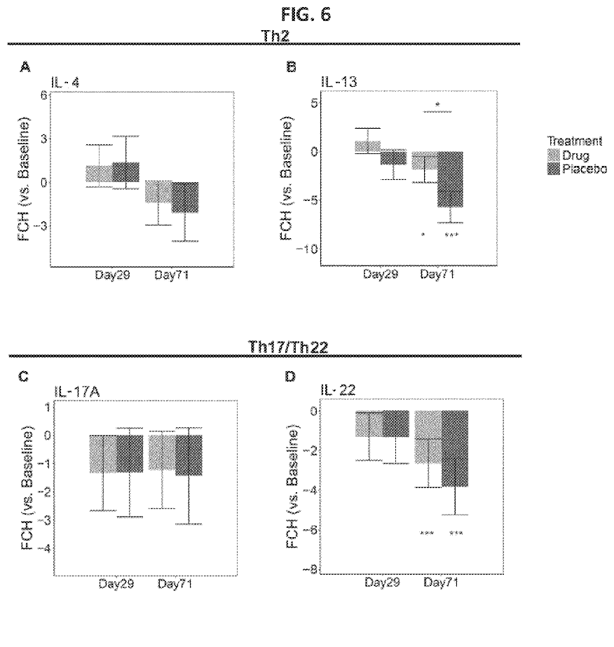

FIG. 6. Th2 and Th17/Th22 markers did not change in GBR830-treated subjects compared to placebo. *P<0.05, ***P<0.001. FCH, fold change.

FIG. 7. Percentage change in EASI from baseline through Day 85. Yellow stars indicate intravenous dose administration. Significance GBR830 vs Placebo: +P<0.1, *P<0.05, **P<0.01. EASI, Eczema Area and Severity Index; ITT, intent to treat; SCORAD, Scoring of Atopic Dermatitis. A. ITT population. B. Severe subjects, SCORAD>50.

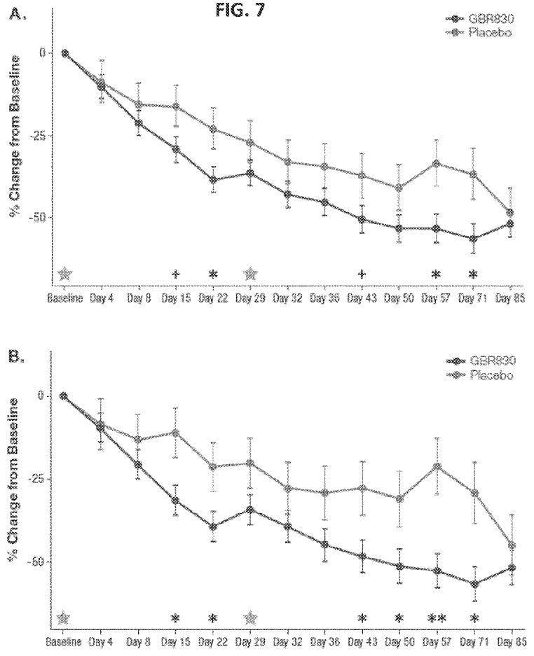

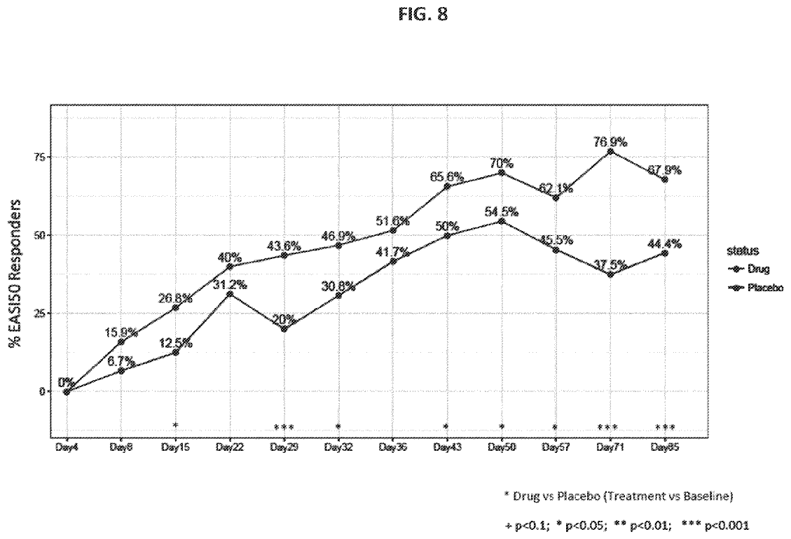

FIG. 8. EASI50 responders. The percentage of EASI50 responders was calculated at different time points for patients treated with the drug and for patients treated with placebo, and plotted. Significance Drug vs Placebo (Treatment vs Baseline): +P<0.1, *P<0.05, **P<0.01, ***P<0.001.

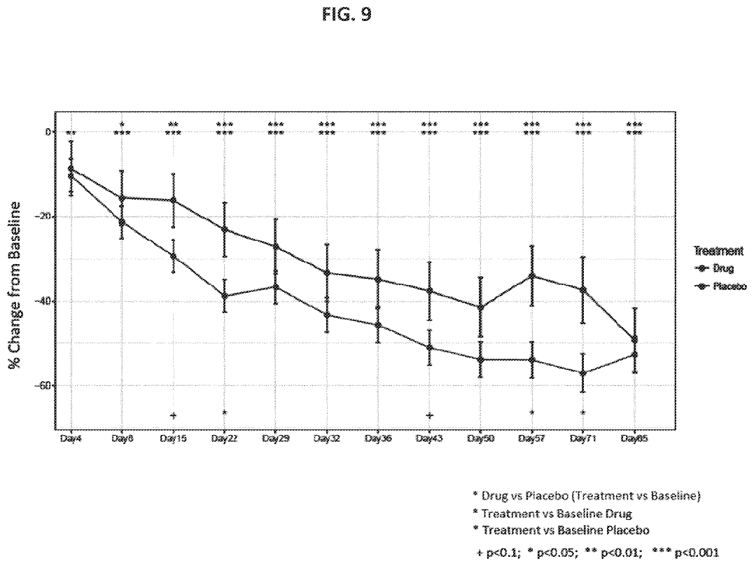

FIG. 9. Improvement EASI scores (%). The EASI score improvement was calculated at different time points for patients treated with the drug and for patients treated with placebo, and plotted. Significance Drug vs Placebo (Treatment vs Baseline): +P<0.1, *P<0.05, **P<0.01, ***P<0.001.

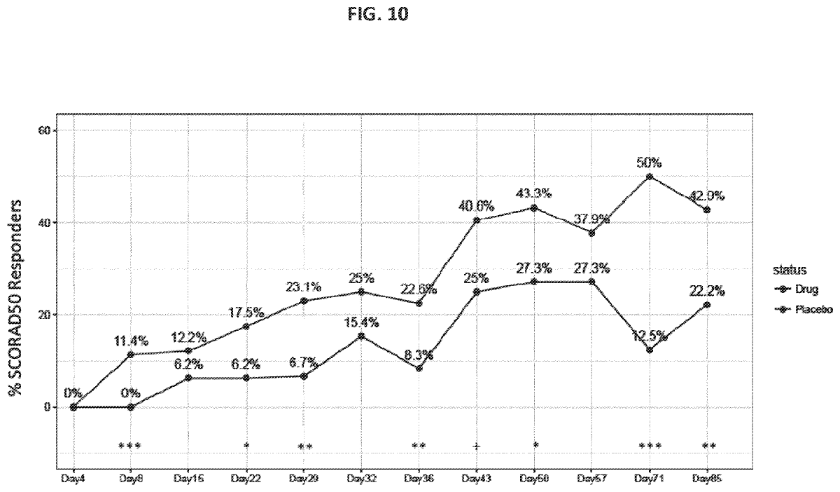

FIG. 10. SCORAD50 responders. The percentage of EASI score improvement was calculated at different time points for patients treated with the drug and for patients treated with placebo, and plotted.

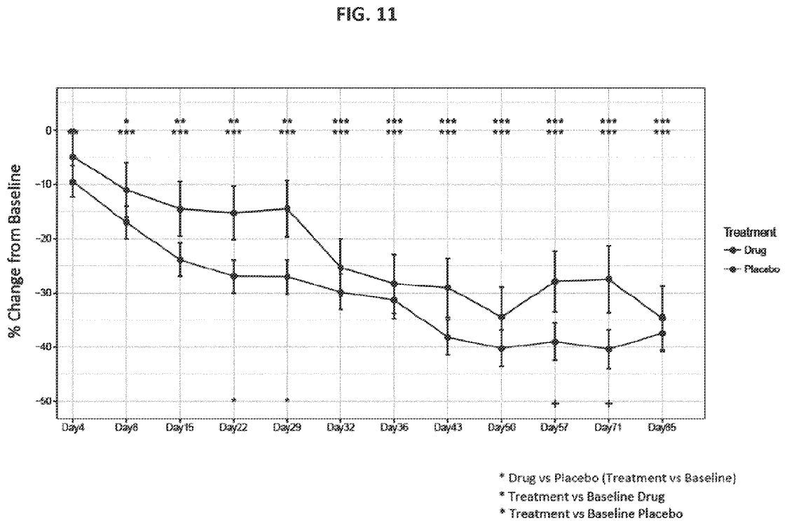

FIG. 11. Improvement SCORAD (%). The SCORAD improvement was calculated at different time points for patients treated with the drug and for patients treated with placebo, and plotted. Significance Drug vs Placebo (Treatment vs Baseline): +P<0.1, *P<0.05, **P<0.01, ***P<0.001.

FIG. 12. Change in Immune markers by RT-PCR. * Significant improvement/lesional characteristics similar to the non-lesional in GBR830 group versus Placebo.

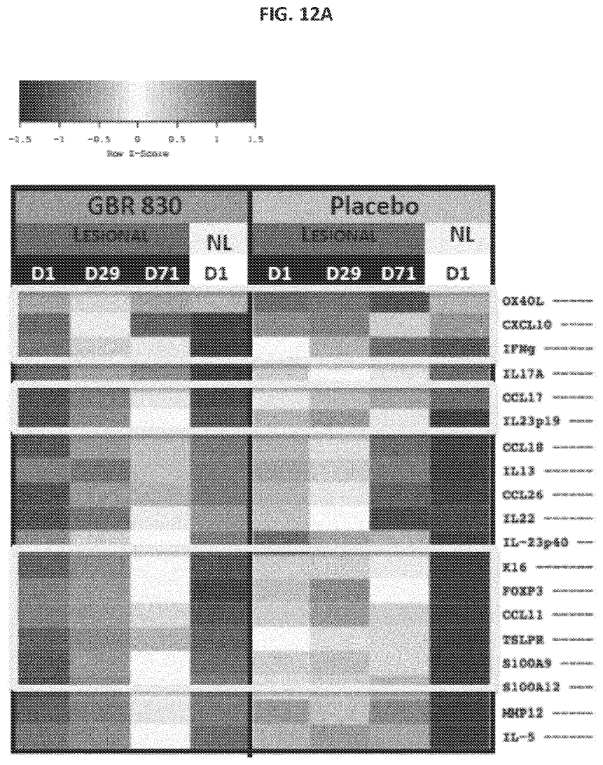

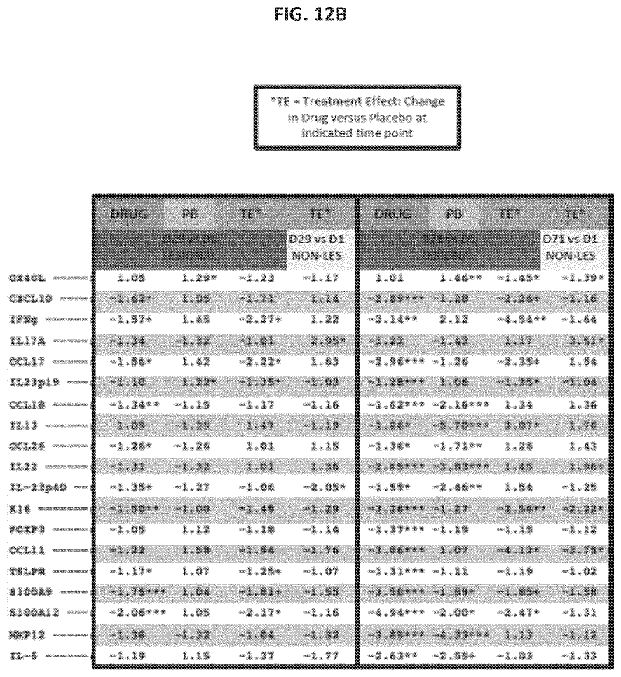

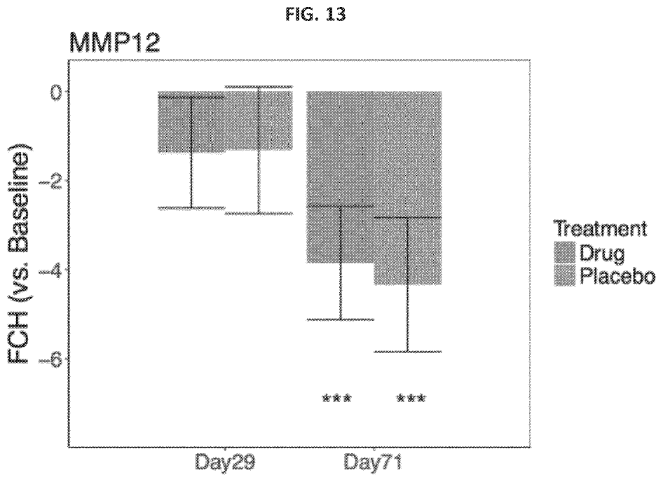

FIG. 13. Inflammatory marker. The inflammatory marker MMP12 was measured for patients treated with the drug and for patients treated with placebo at Day 29 and at Day 71.

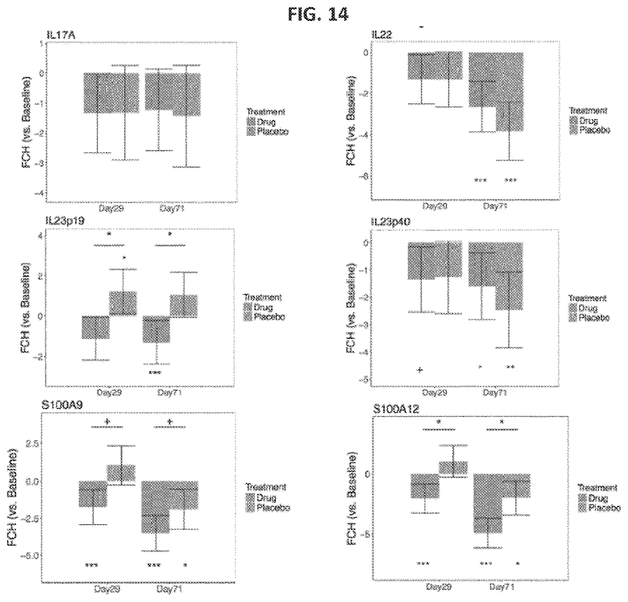

FIG. 14. Th17/Th22 related cytokines. Th17/Th22 related cytokines were measured for patients treated with the drug and for patients treated with placebo at Day 29 and at Day 71. Top-left panel: IL17A; top-right panel: IL22; middle-left panel: IL23p19; middle-right panel: IL23p40; bottom-left panel: S100A9; bottom-right panel: S100A12.

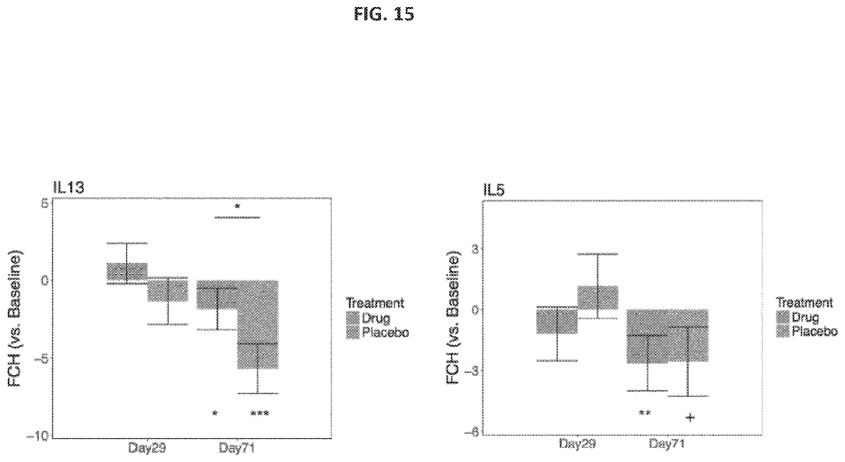

FIG. 15. Th2 specific cytokines. The Th2 specific cytokines IL13 (left panel) and IL5 (right panel) were measured for patients treated with the drug and for patients treated with placebo at Day 29 and at Day 71.

FIG. 16. Th2 specific chemokines. Th2 specific chemokines were measured for patients treated with the drug and for patients treated with placebo at Day 29 and at Day 71. Top-left panel: CCL11; top-right panel: CCL17; middle-left panel: CCL18; middle-right panel: CCL26; bottom-left panel: OX40L; bottom-right panel: TSLPR.

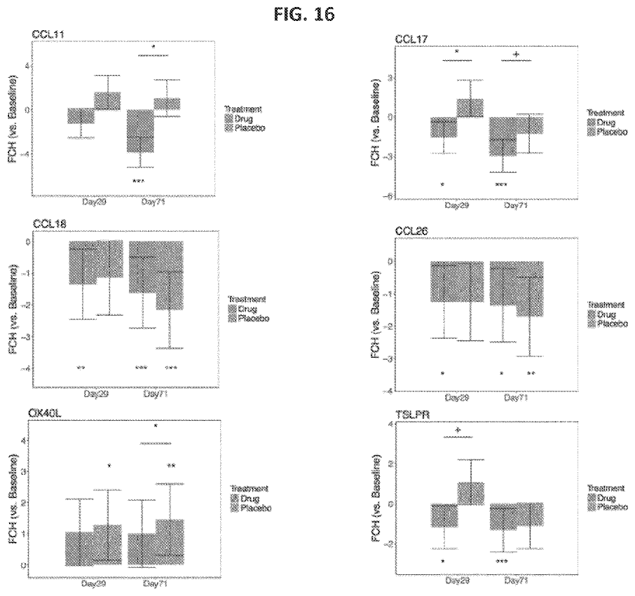

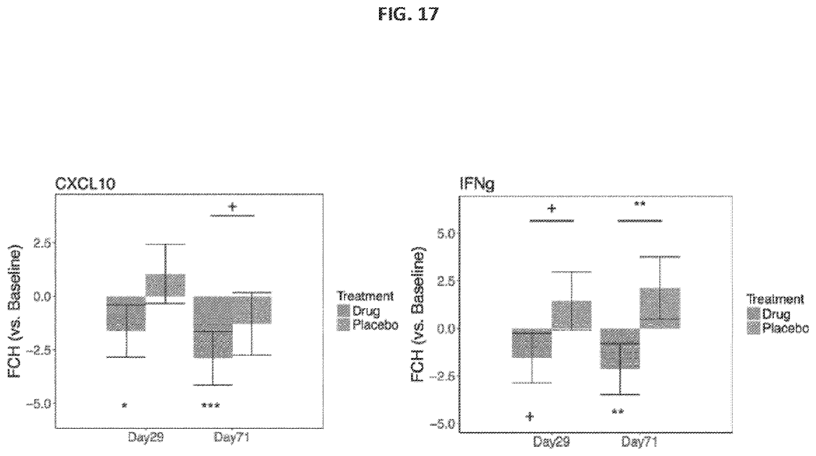

FIG. 17. Th1/IFN related Immune mediators. The Th1/IFN related Immune mediators CXCL10 (left panel) and IFNg (right panel) were measured for patients treated with the drug and for patients treated with placebo at Day 29 and at Day 71.

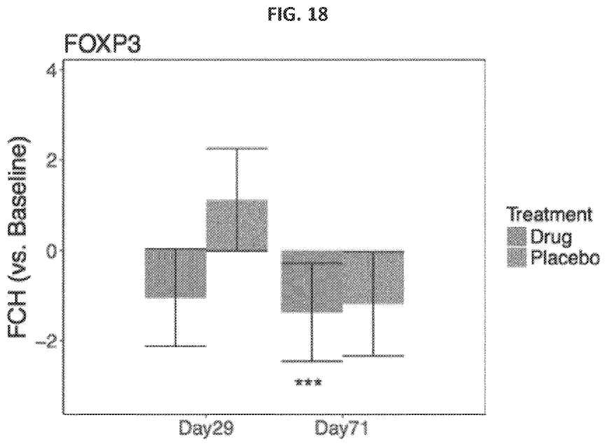

FIG. 18. Treg specific immune mediator. The Treg specific immune mediator FOXP3, was measured for patients treated with the drug and for patients treated with placebo at Day 29 and at Day 71.

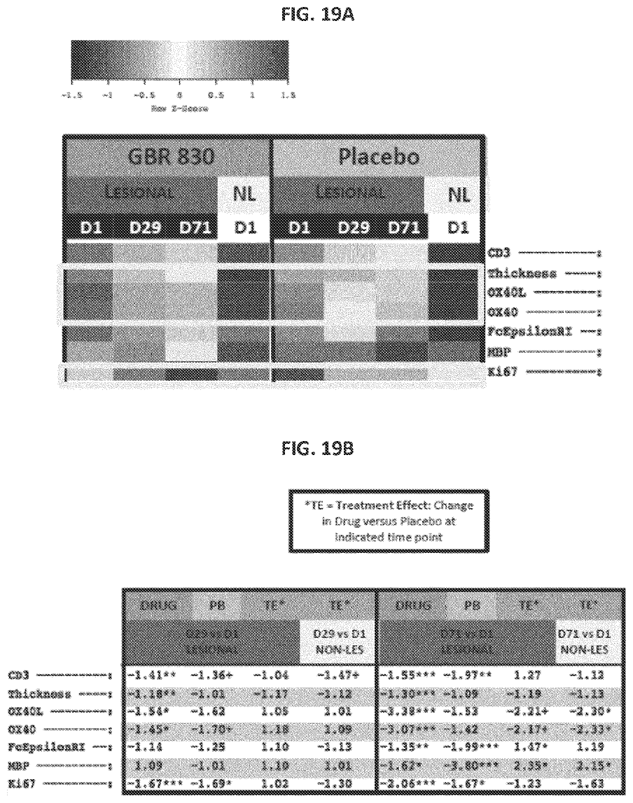

FIG. 19. Immune markers profiled by IHC. * Significant improvement in treatment group vs placebo group in OX40/OX40L and hyperplasia markers (Ki67, thickness)

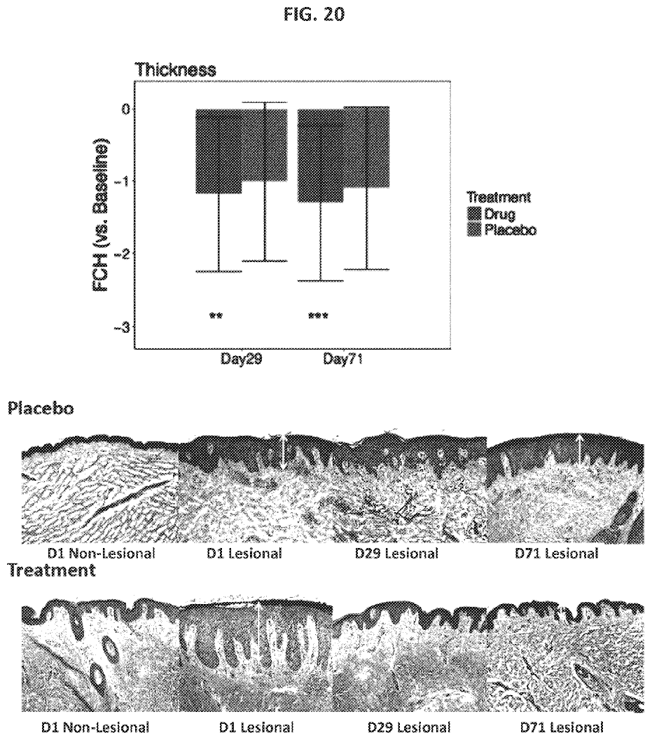

FIG. 20. Hyperplasia markers—H&E thickness. Immunohistochemistry images for representative drug and placebo subjects, at baseline and after treatment, and quantification of thickness (top panel)

FIG. 21. Hyperplasia markers—Ki67. Immunohistochemistry images for representative drug and placebo subjects, at baseline and after treatment, Ki67 staining and quantification (top panel).

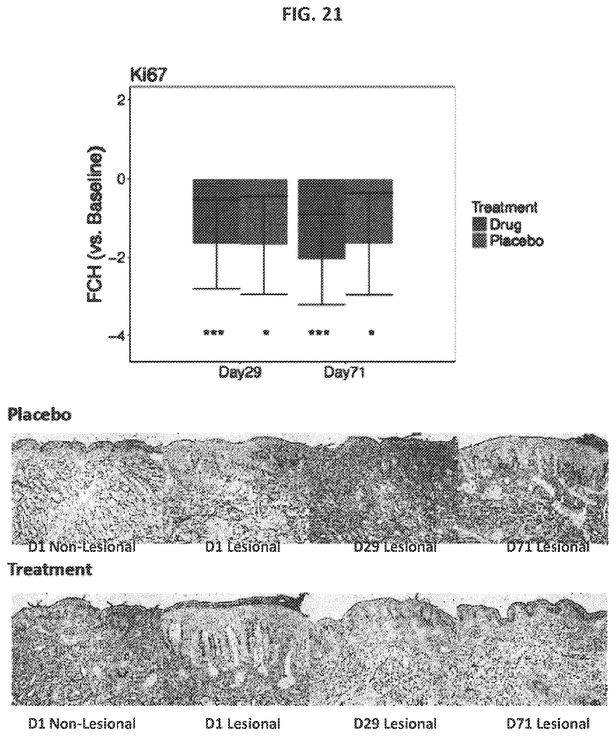

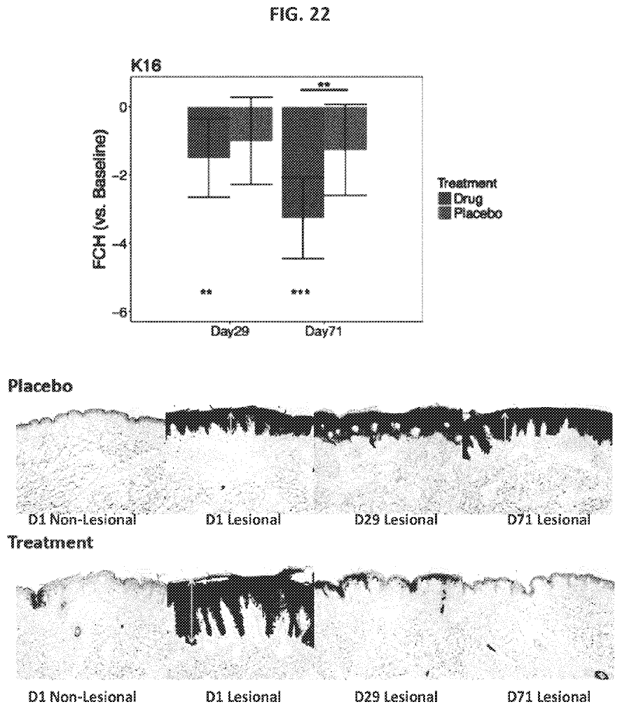

FIG. 22. Hyperplasia markers—K16. Immunohistochemistry images for representative drug and placebo subjects, at baseline and after treatment, K16 staining and quantification (top panel).

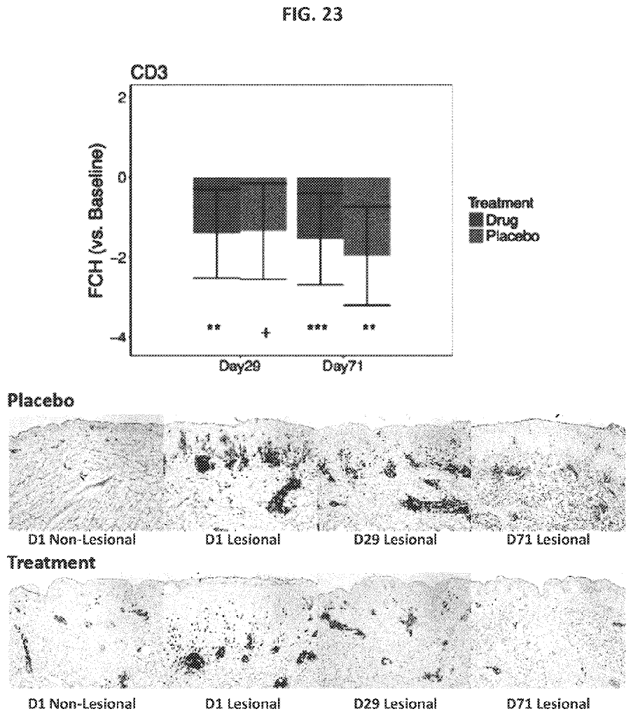

FIG. 23. Cellular infiltrate—T-cells (CD3). Immunohistochemistry images for representative drug and placebo subjects, at baseline and after treatment, CD3 staining and quantification (top panel).

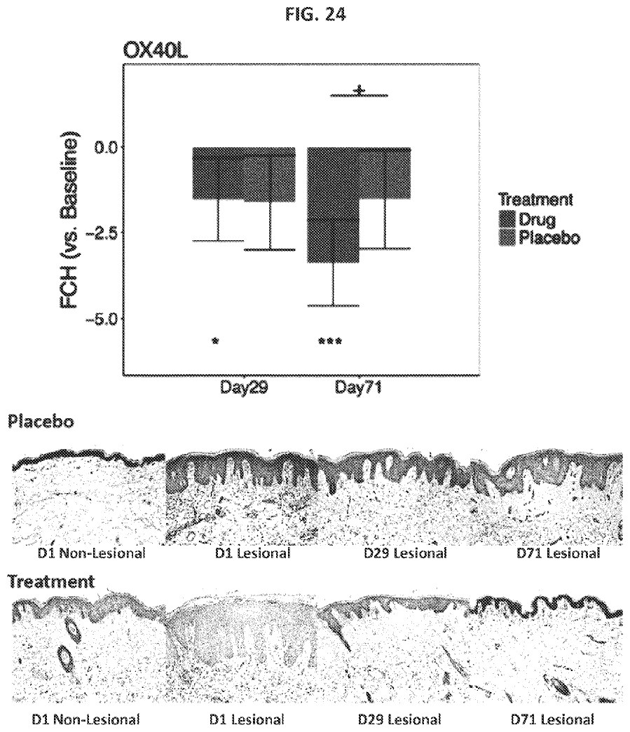

FIG. 24. Cellular infiltrate—Atopic dendritic cells (OX40L). Immunohistochemistry images for representative drug and placebo subjects, at baseline and after treatment, OX40L staining and quantification (top panel).

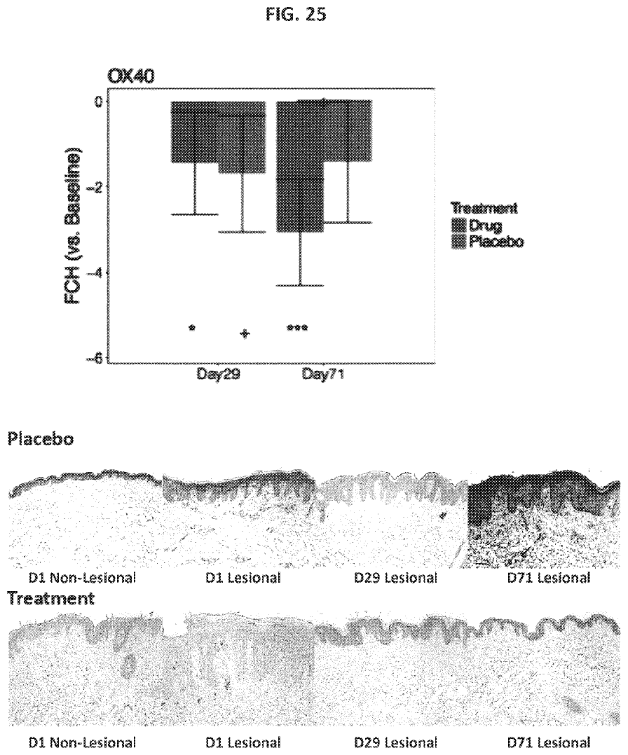

FIG. 25. Cellular infiltrate—OX40+ T cells. Immunohistochemistry images for representative drug and placebo subjects, at baseline and after treatment, OX40 staining and quantification (top panel).

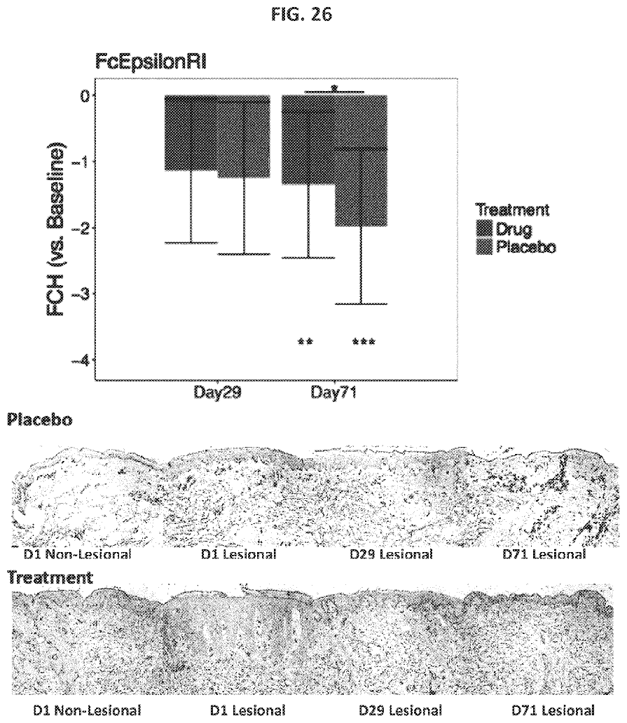

FIG. 26. Cellular infiltrate—Inflammatory Dendritic Epidermal Cells (IDECs). Immunohistochemistry images for representative drug and placebo subjects, at baseline and after treatment, and FcEpsilonRI quantification (top panel).

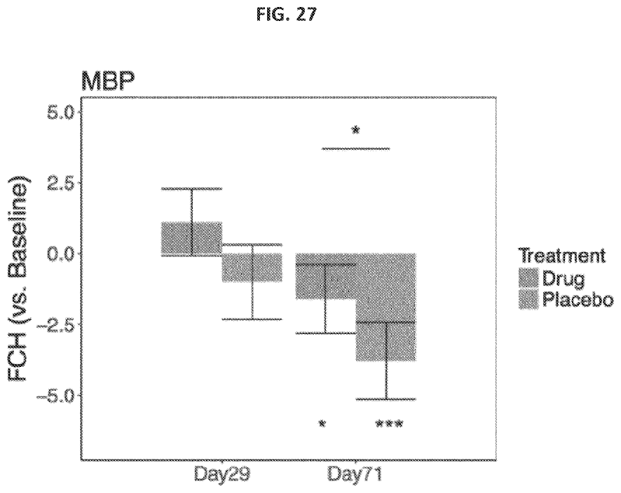

FIG. 27. Cellular infiltrate—Eosinophils (MBP). The eosinophils MBP was measured for patients treated with the drug and for patients treated with placebo at Day 29 and at Day 71.

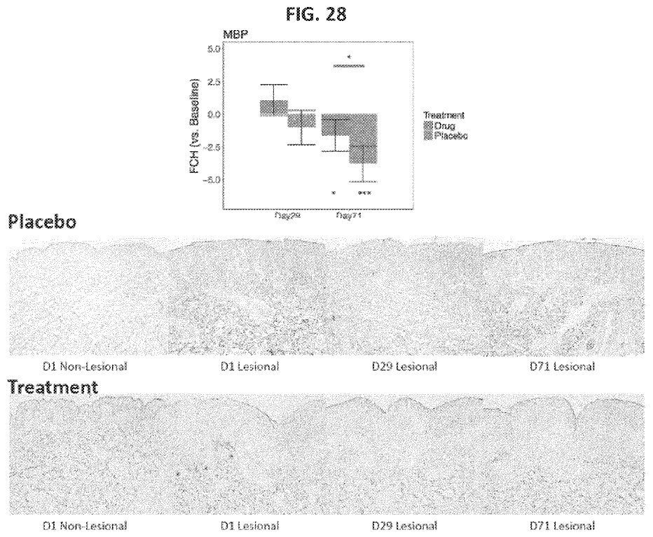

FIG. 28. Cellular infiltrate—Eosinophils (MBP). Immunohistochemistry images for representative drug and placebo subjects, at baseline and after treatment, and MBP quantification (top panel).

FIG. 29. IL17A. Responders subanalysis (RT-PCR data) for IL17A. Top-left panel: EASI50; top-right panel: EASI75; bottom-left panel: histological responders.

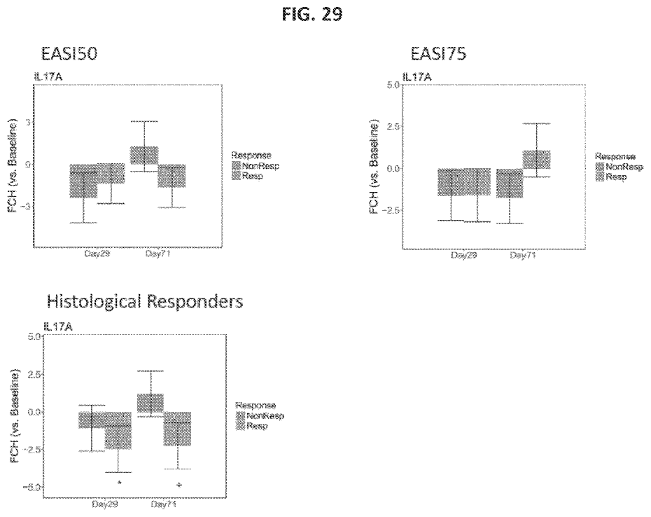

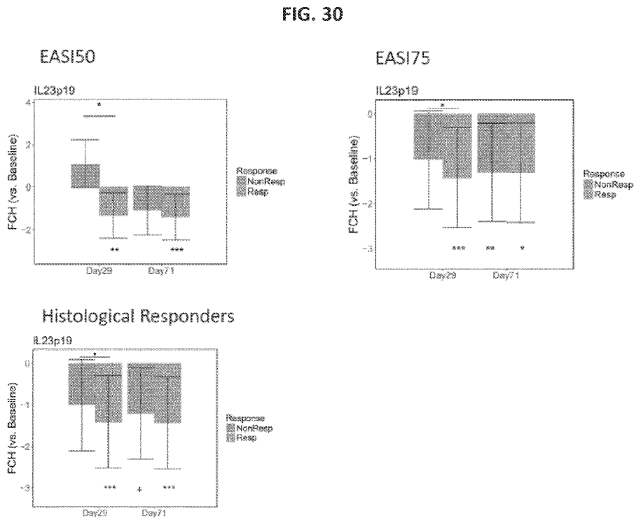

FIG. 30. IL23p19. Responders subanalysis (RT-PCR data) for IL23p19. Top-left panel: EASI50; top-right panel: EASI75; bottom-left panel: histological responders.

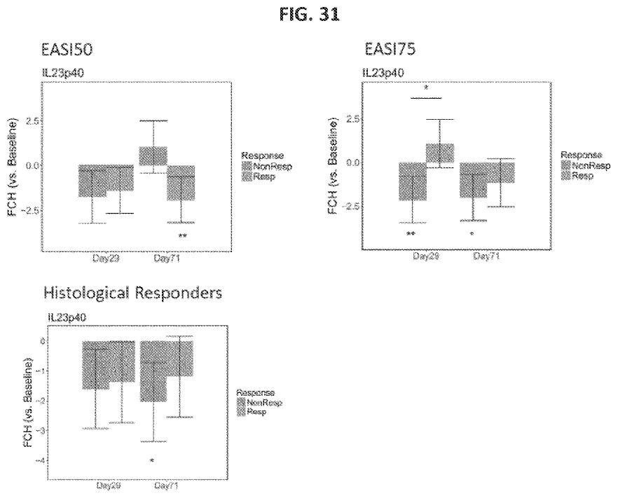

FIG. 31. IL23p40. Responders subanalysis (RT-PCR data) for IL23p40. Top-left panel: EASI50; top-right panel: EASI75; bottom-left panel: histological responders.

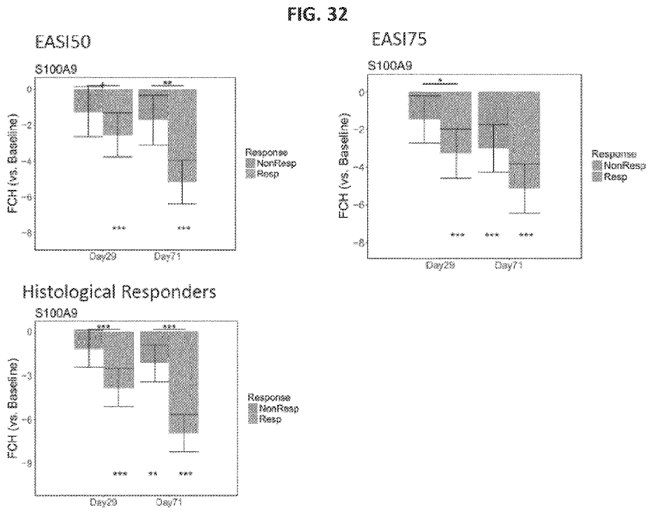

FIG. 32. S100A9. Responders subanalysis (RT-PCR data) for $100A9. Top-left panel: EASI50; top-right panel: EASI75; bottom-left panel: histological responders.

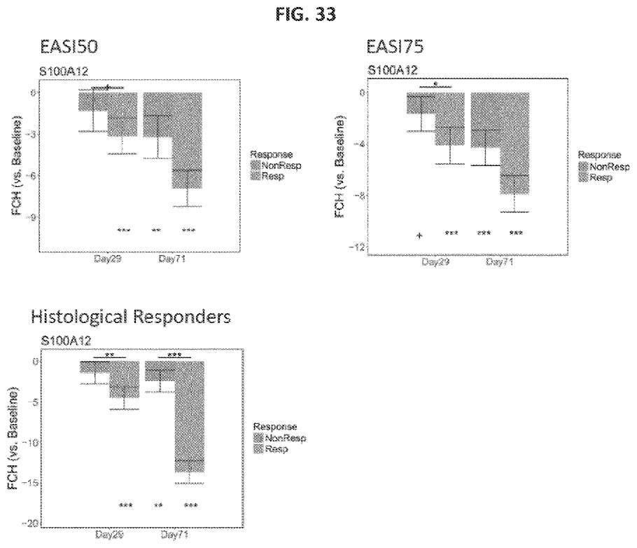

FIG. 33. S100A12. Responders subanalysis (RT-PCR data) for S100A12. Top-left panel: EASI50; top-right panel: EASI75; bottom-left panel: histological responders.

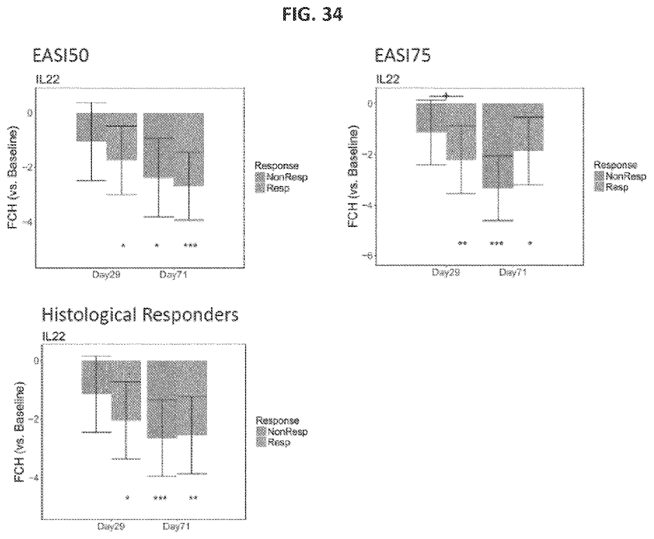

FIG. 34. IL22. Responders subanalysis (RT-PCR data) for IL22. Top-left panel: EASI50; top-right panel: EASI75; bottom-left panel: histological responders.

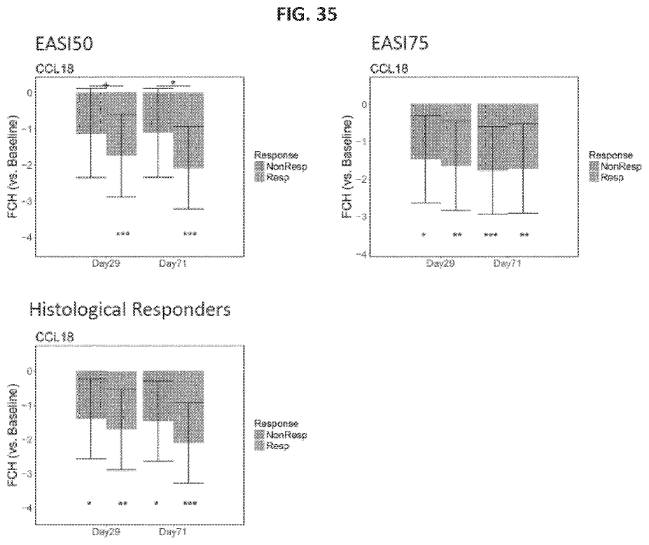

FIG. 35. CCL18. Responders subanalysis (RT-PCR data) for CCL18. Top-left panel: EASI50; top-right panel: EASI75; bottom-left panel: histological responders.

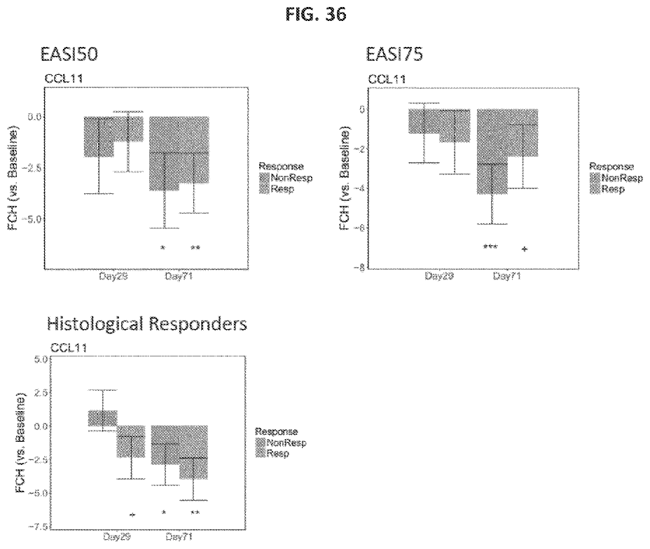

FIG. 36. CCL11. Responders subanalysis (RT-PCR data) for CCL11. Top-left panel: EASI50; top-right panel: EASI75; bottom-left panel: histological responders.

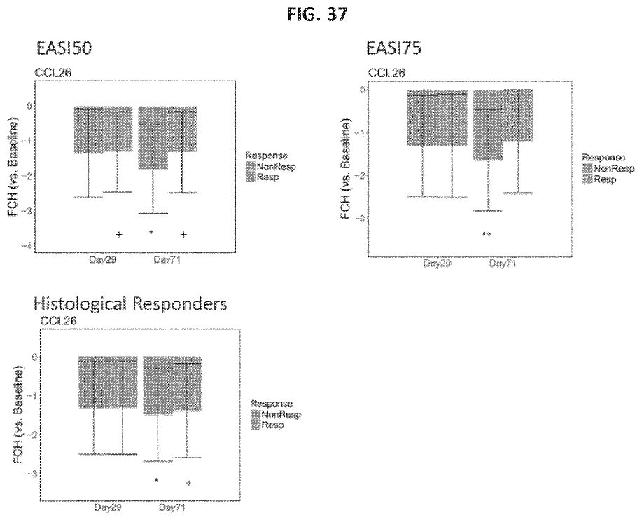

FIG. 37. CCL26. Responders subanalysis (RT-PCR data) for CCL26. Top-left panel: EASI50; top-right panel: EASI75; bottom-left panel: histological responders.

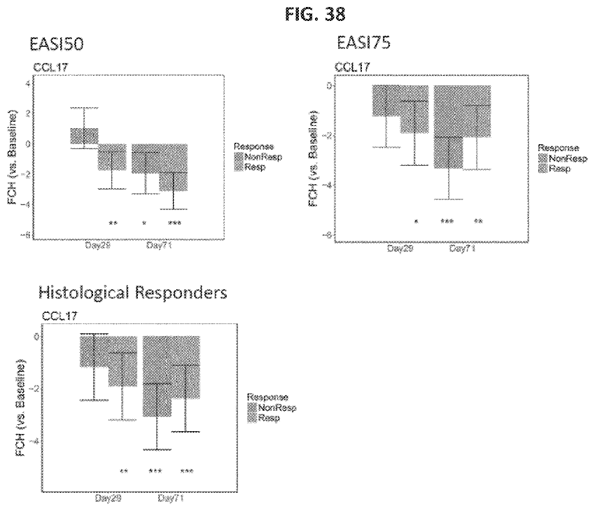

FIG. 38. CCL17. Responders subanalysis (RT-PCR data) for CCL17. Top-left panel: EASI50; top-right panel: EASI75; bottom-left panel: histological responders.

FIG. 39. TSLPR. Responders subanalysis (RT-PCR data) for TSLPR. Top-left panel: EASI50; top-right panel: EASI75; bottom-left panel: histological responders.

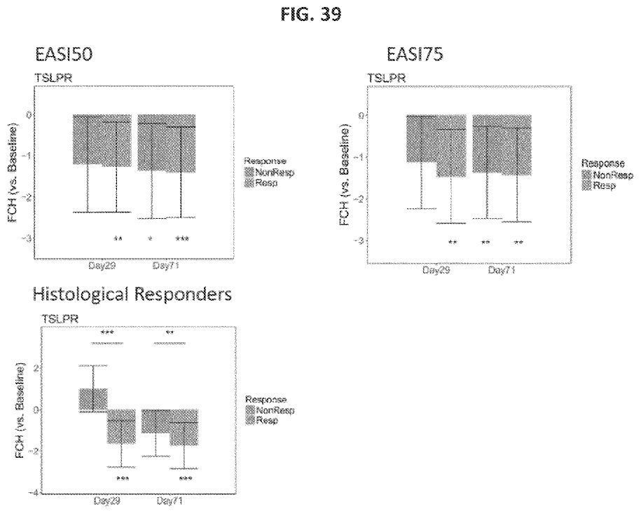

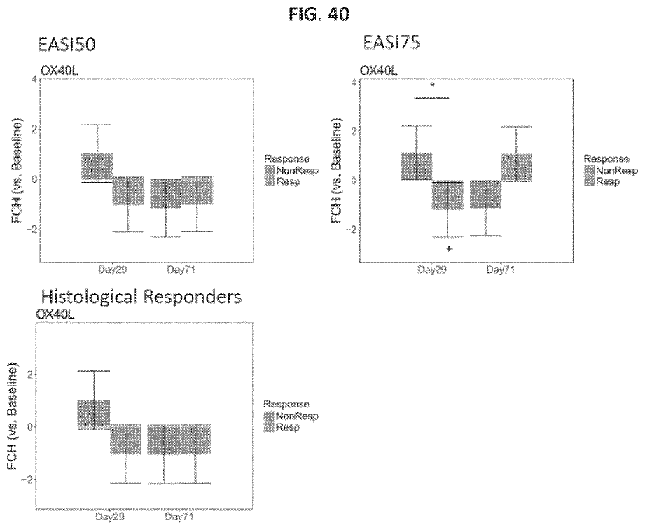

FIG. 40. OX40L. Responders subanalysis (RT-PCR data) for OX40L. Top-left panel: EASI50; top-right panel: EASI75; bottom-left panel: histological responders.

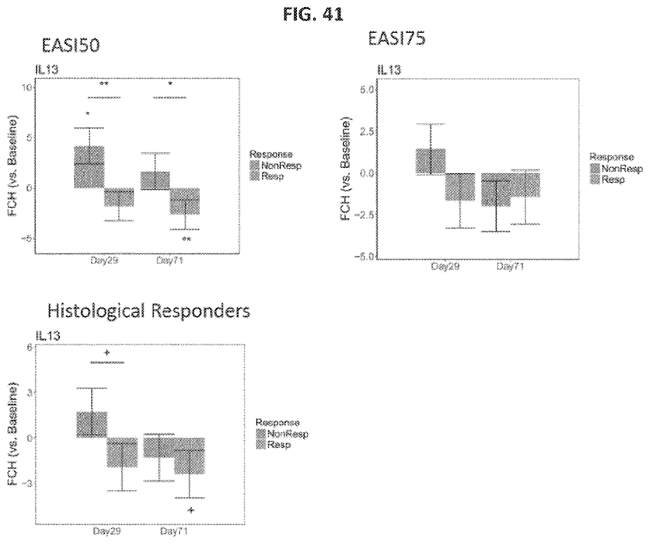

FIG. 41. IL.13. Responders subanalysis (RT-PCR data) for IL13. Top-left panel: EASI50; top-right panel: EASI75; bottom-left panel: histological responders.

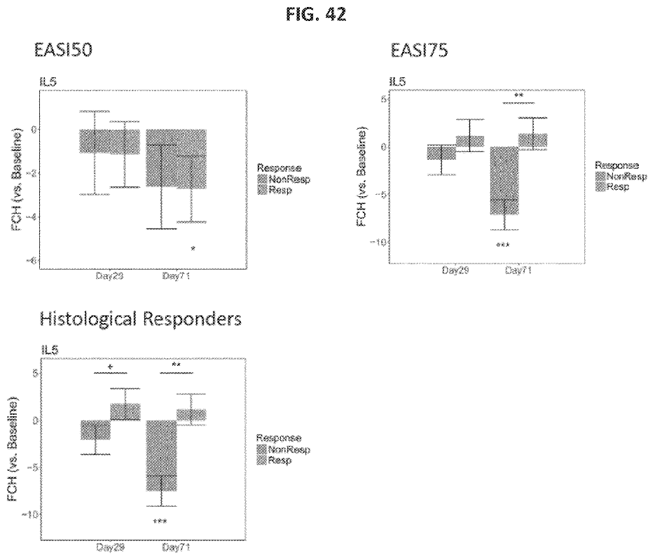

FIG. 42. IL5. Responders subanalysis (RT-PCR data) for ILS. Top-left panel: EASI50; top-right panel: EASI75; bottom-left panel: histological responders.

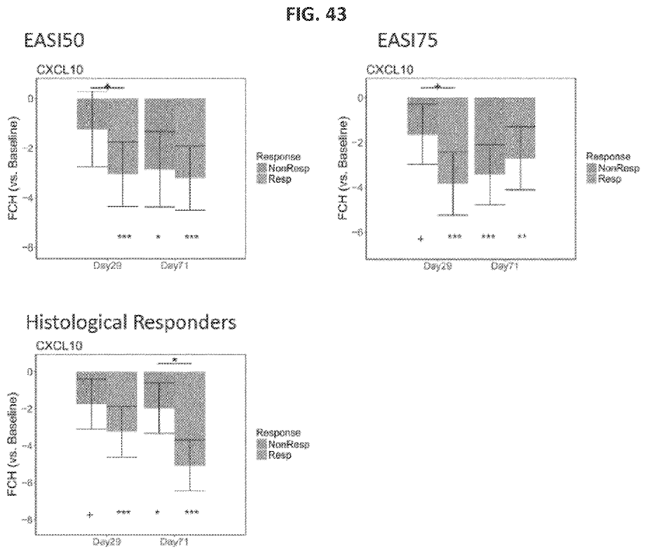

FIG. 43. CXCL10. Responders subanalysis (RT-PCR data) for CXCL10. Top-left panel: EASI50; top-right panel: EASI75; bottom-left panel: histological responders.

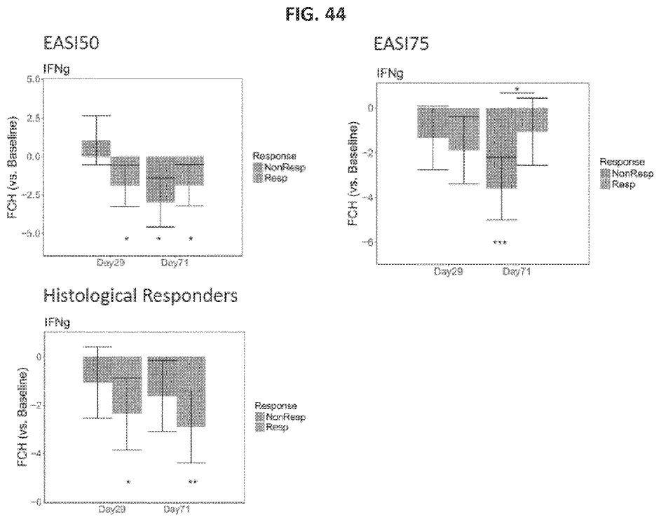

FIG. 44. IFNg. Responders subanalysis (RT-PCR data) for IFNg. Top-left panel: EASI50; top-right panel: EASI75; bottom-left panel: histological responders.

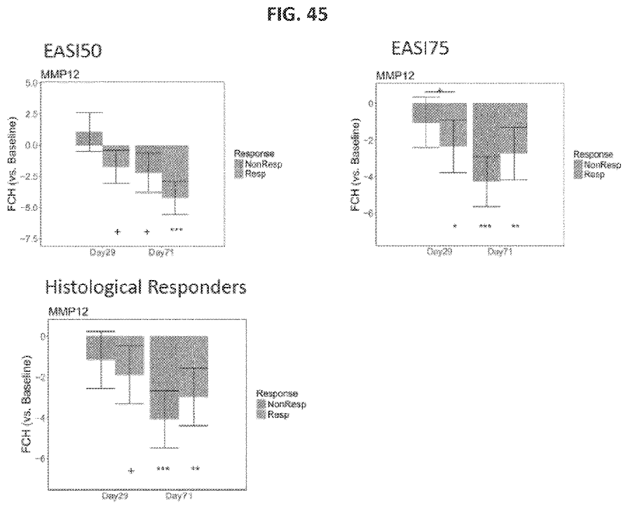

FIG. 45. Inflammatory marker MMP12. Responders subanalysis (RT-PCR data) for MMP12. Top-left panel: EASI50; top-right panel: EASI75; bottom-left panel: histological responders.

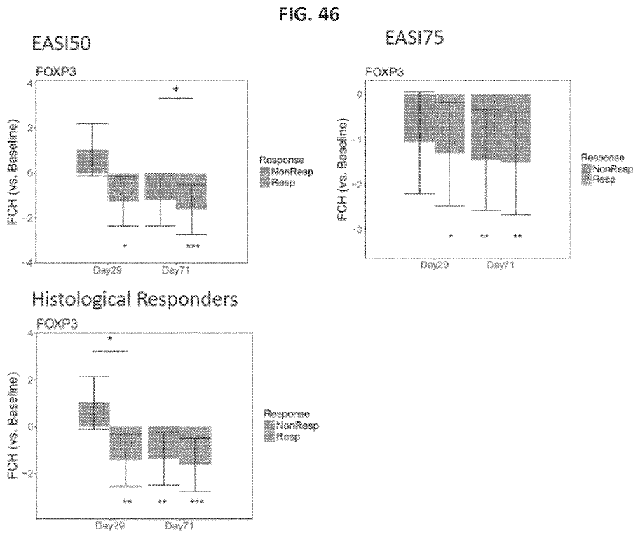

FIG. 46. Treg specific immune mediator FOXP3. Responders subanalysis (RT-PCR data) for FOXP3. Top-left panel: EASI50; top-right panel: EASI75; bottom-left panel: histological responders.

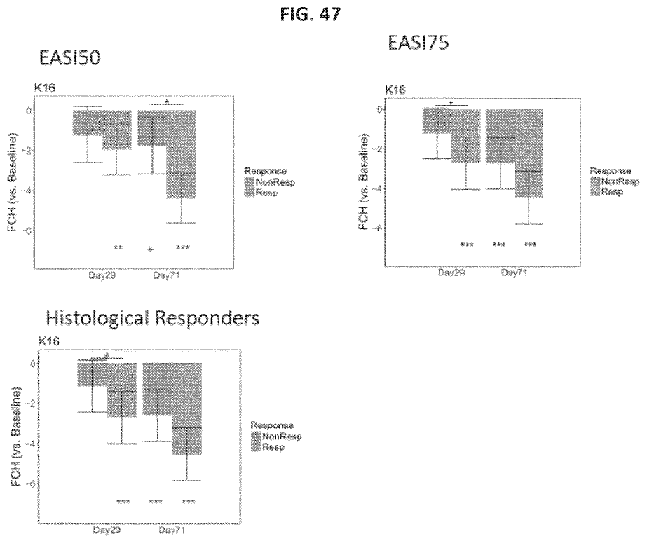

FIG. 47. Hyperplasia Marker K16. Responders subanalysis (RT-PCR data) for K16. Top-left panel: EASI50; top-right panel: EASI75; bottom-left panel: histological responders.

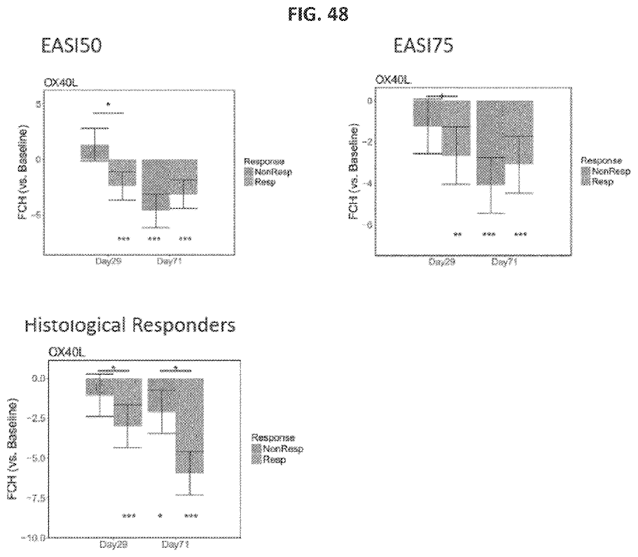

FIG. 48. Atopic dendritic cells OX40L. Responders subanalysis (RT-PCR data) for OX40L. Top-left panel: EASI50; top-right panel: EASI75; bottom-left panel: histological responders.

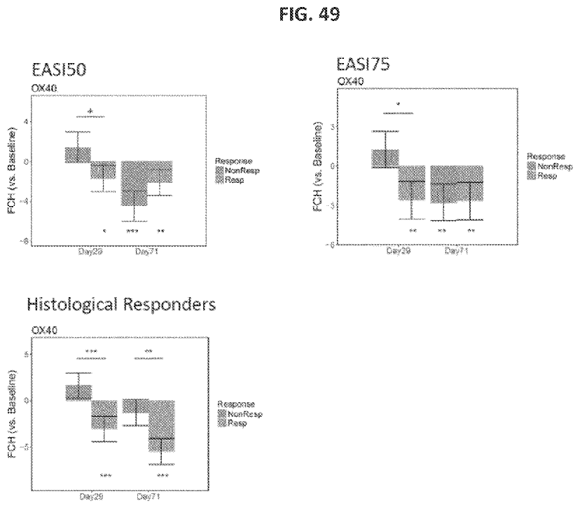

FIG. 49. OX40+ T cells OX40. Responders subanalysis (RT-PCR data) for OX40. Top-left panel: EASI50; top-right panel: EASI75; bottom-left panel: histological responders.

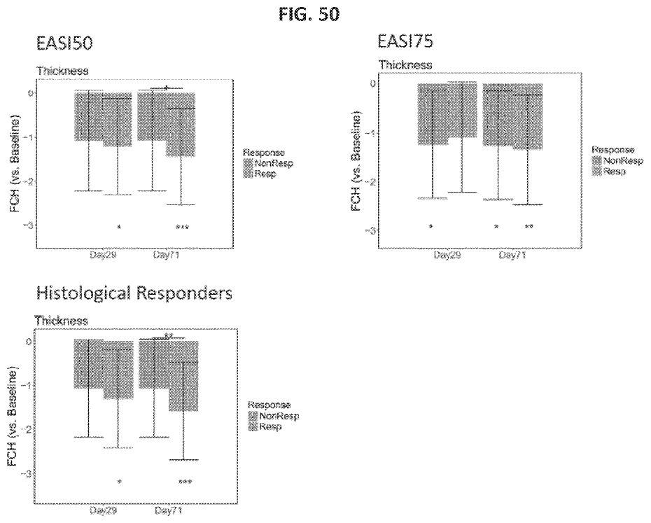

FIG. 50. Thickness. Responders subanalysis (RT-PCR data) for Thickness. Top-left panel: EASI50; top-right panel: EASI75; bottom-left panel: histological responders.

FIG. 51. Ki67. Responders subanalysis (RT-PCR data) for Ki67. Top-left panel: EASI50; top-right panel: EASI75; bottom-left panel: histological responders.

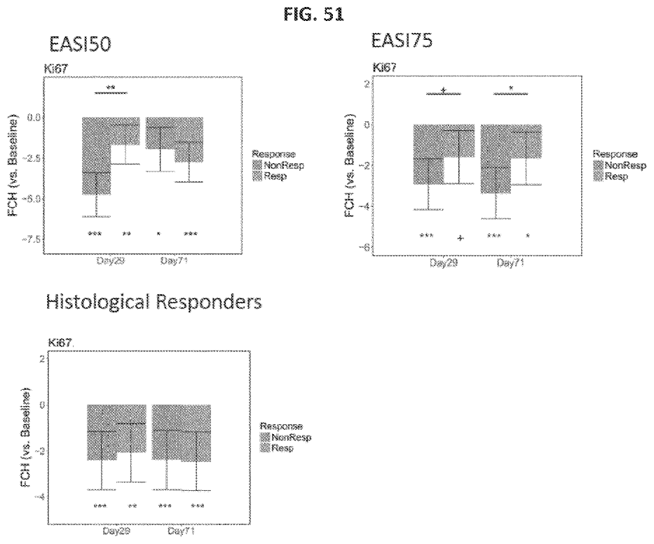

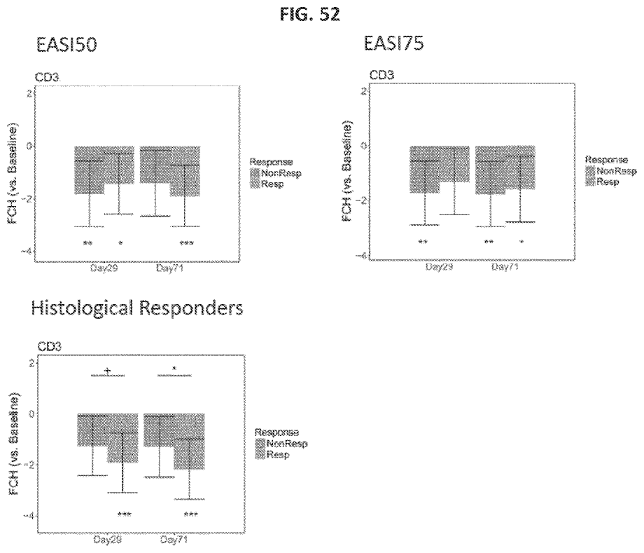

FIG. 52. T-cell marker CD3. Responders subanalysis (RT-PCR data) for CD3. Top-left panel: EASI50; top-right panel: EASI75; bottom-left panel: histological responders.

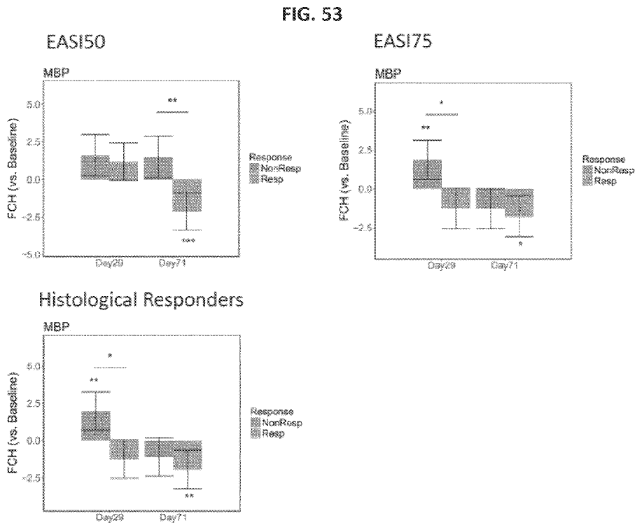

FIG. 53. Eosinophil marker MBP. Responders subanalysis (RT-PCR data) for MBP. Top-left panel: EASI50; top-right panel: EASI75; bottom-left panel: histological responders.

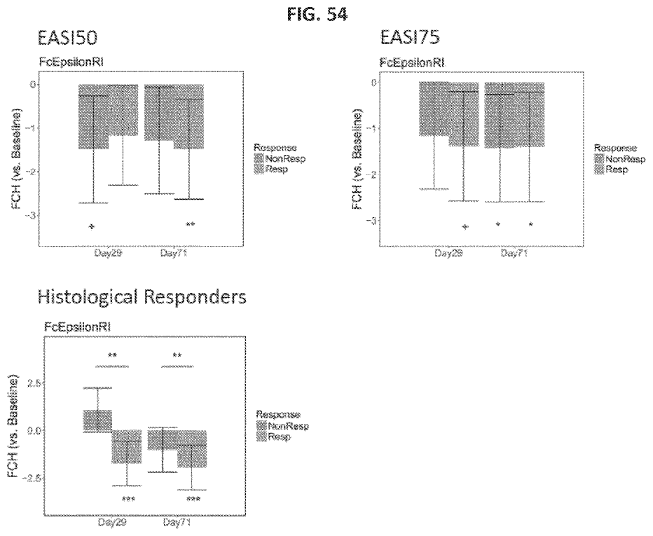

FIG. 54. Inflammatory dendritic epidermal cell (IDECs) FcEpsilonRI. Responders subanalysis (RT-PCR data) for FcEpsilonRI. Top-left panel: EASI50; top-right panel: EASI75; bottom-left panel: histological responders.

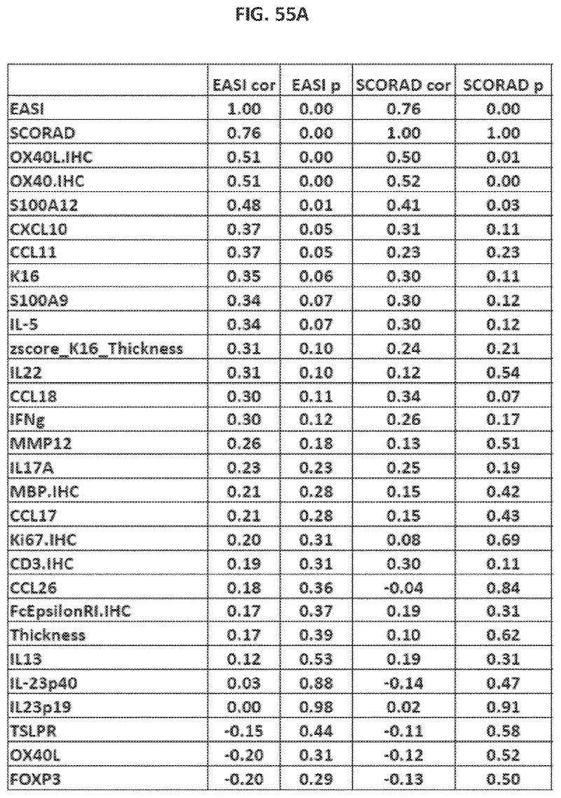

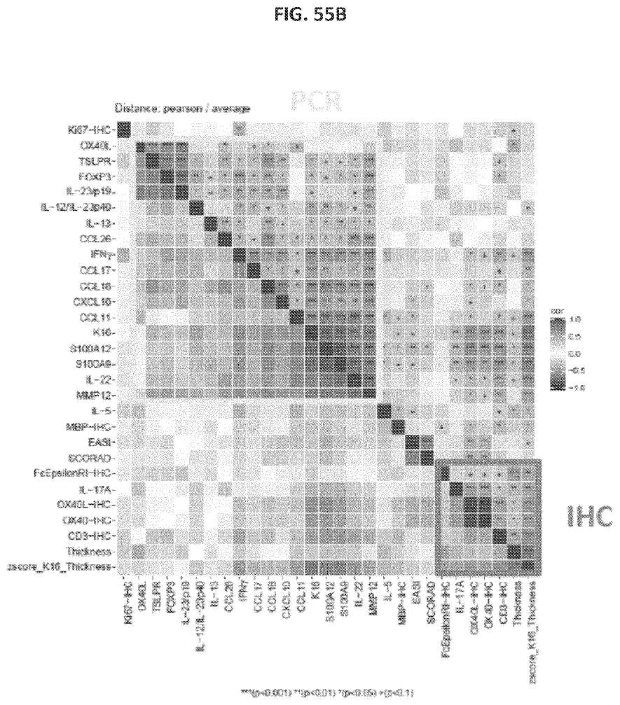

FIG. 55. Correlation at baseline. Correlation between biomarkers was analyzed at baseline.

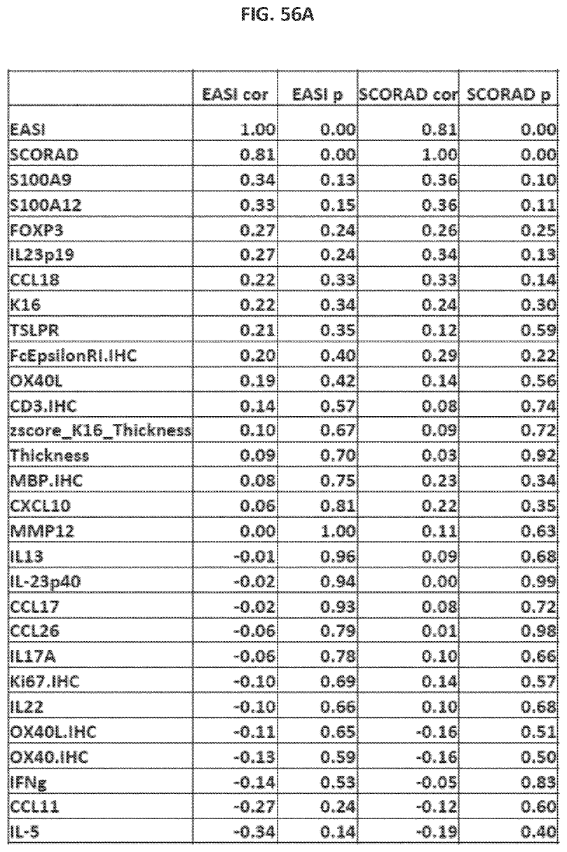

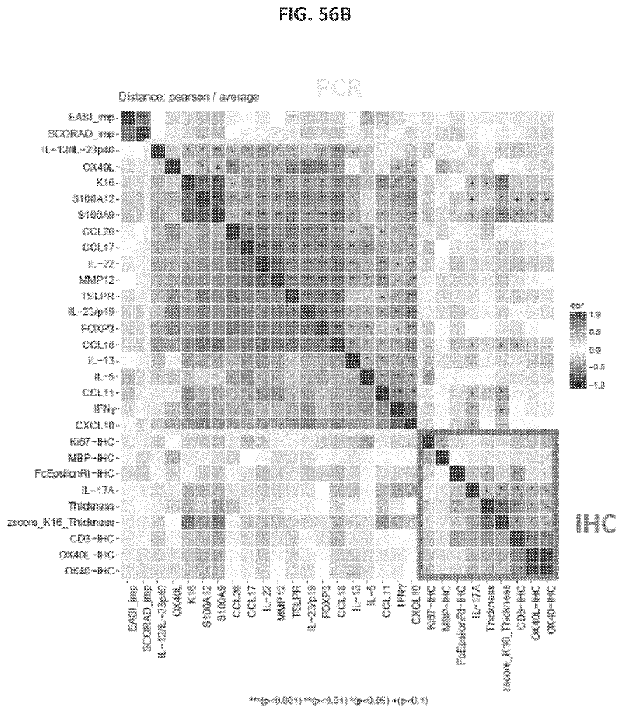

FIG. 56. Correlation of improvement at Day 71. Correlation between biomarkers was calculated at Day 71.

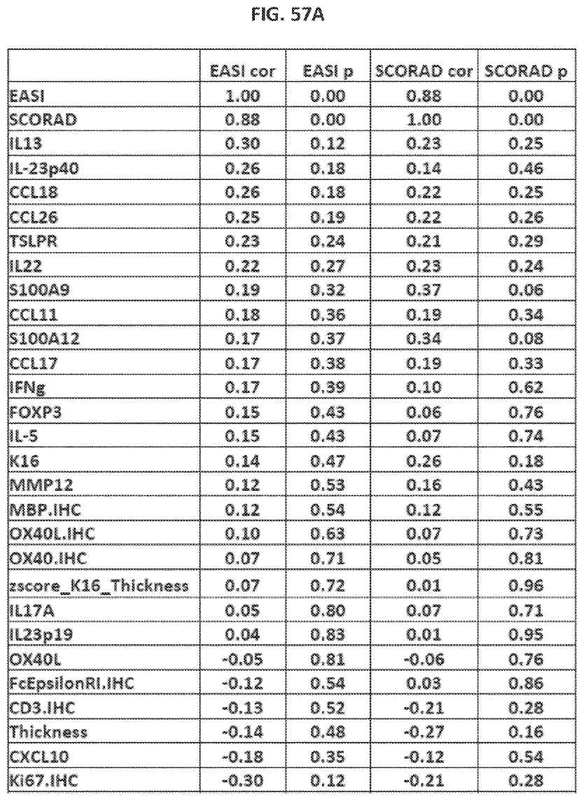

FIG. 57. Correlation of improvemtn at Day 29. Correlation between biomarkers was calculated at Day 29.

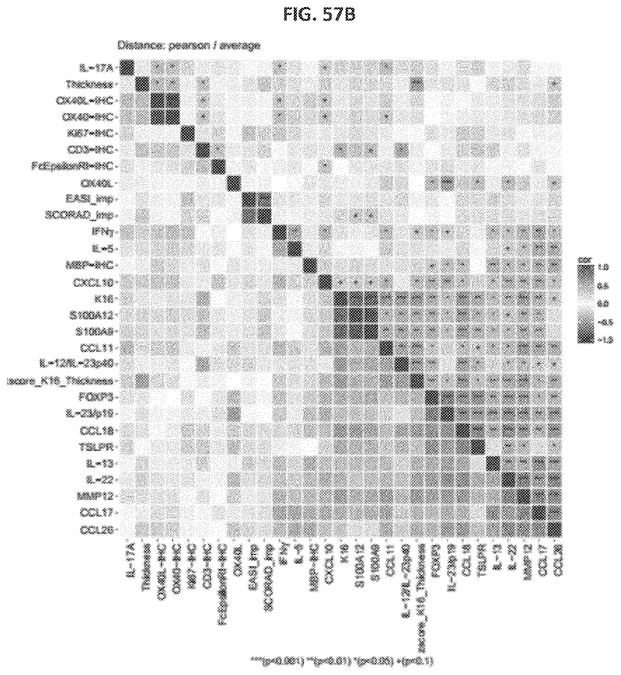

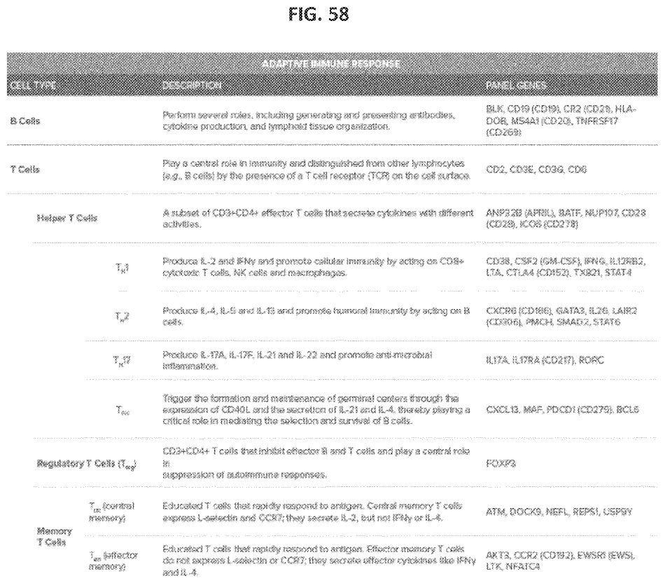

FIG. 58. Brief description of the immune cell types and selected genes included in the PanCancer Immune Profiling Panel

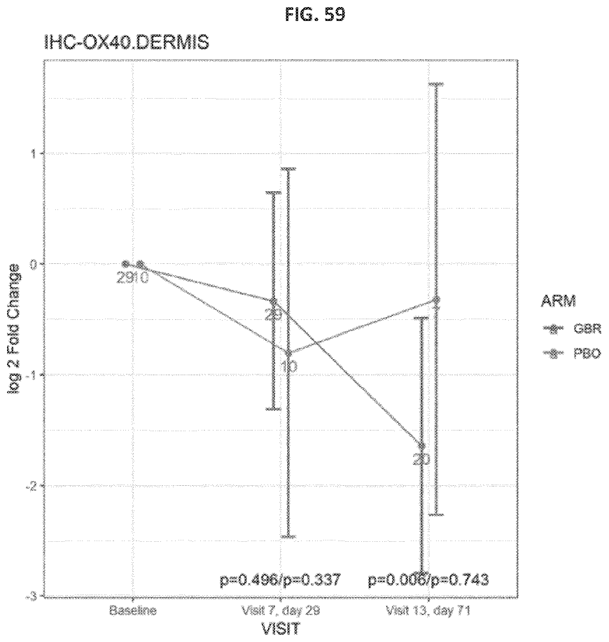

FIG. 59. Least squared means estimates for OX40. DERMIS

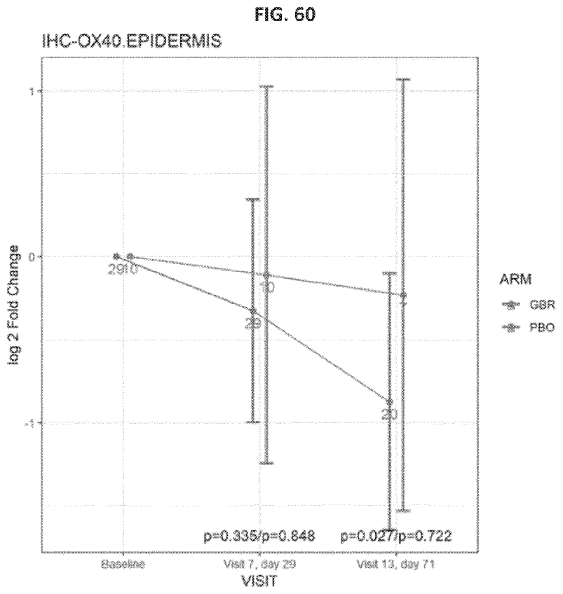

FIG. 60. Least squared means estimates for OX40. EPIDERMIS

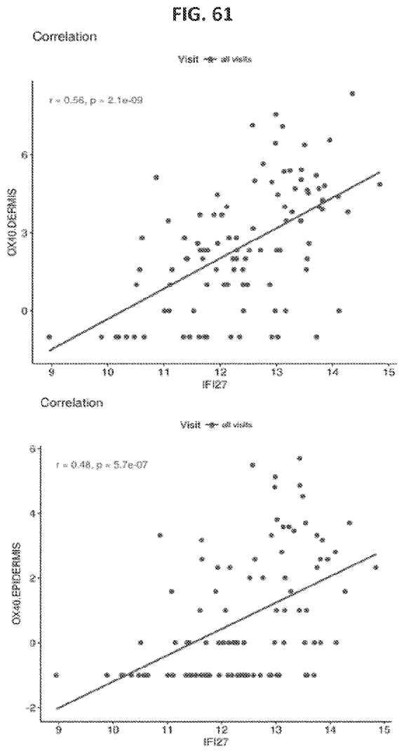

FIG. 61. Correlation between IFI27 and OX40 expression

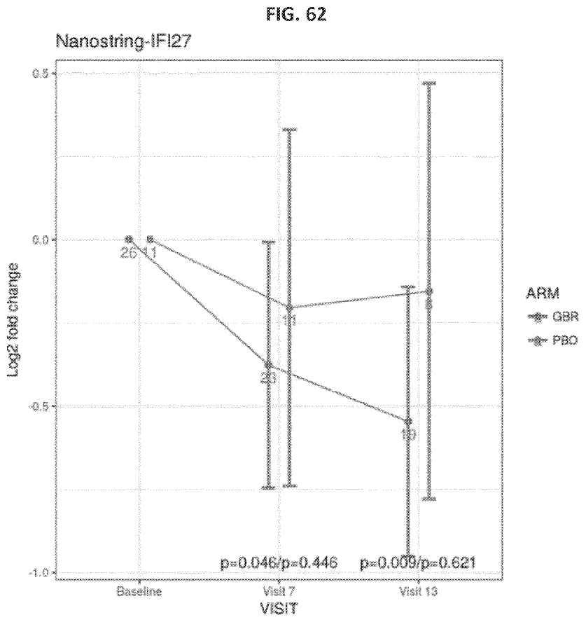

FIG. 62. Least squared means estimates for IFI27

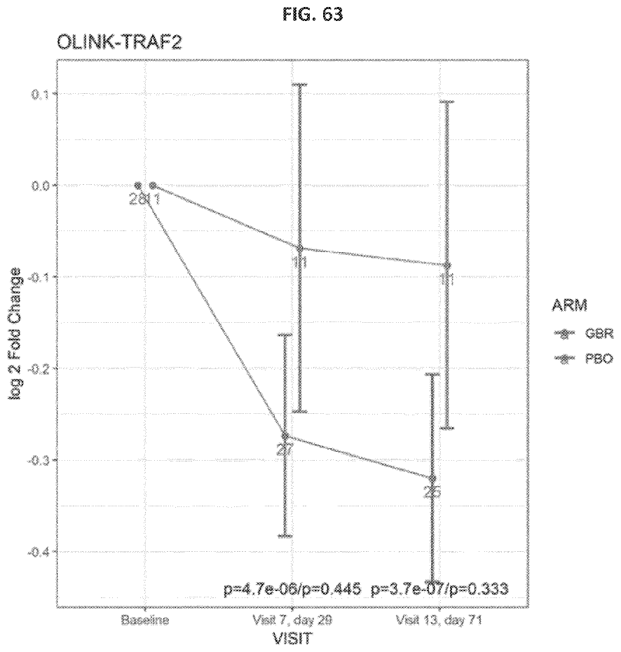

FIG. 63. Least squared means estimates for TRAF2 (OLINK)

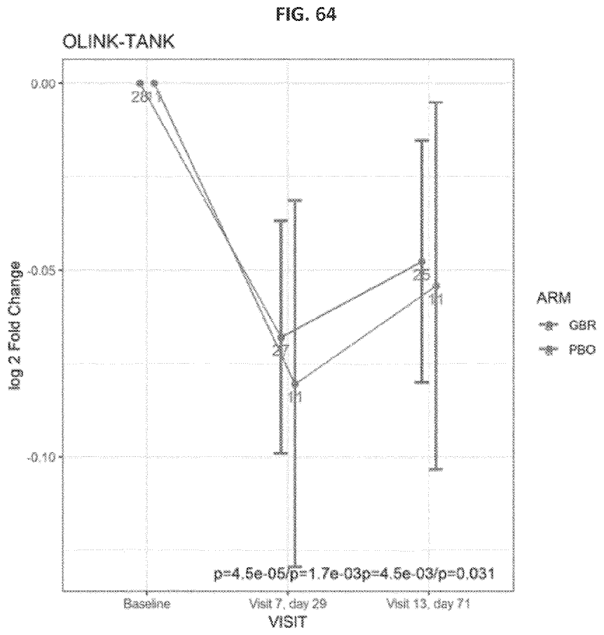

FIG. 64. Least squared means estimates for TANK (OLINK)

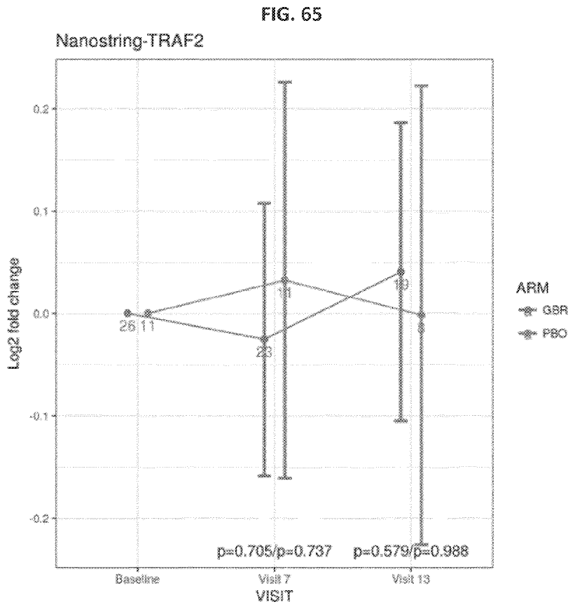

FIG. 65. Least squared means estimates for TRAF2 (NanoString)

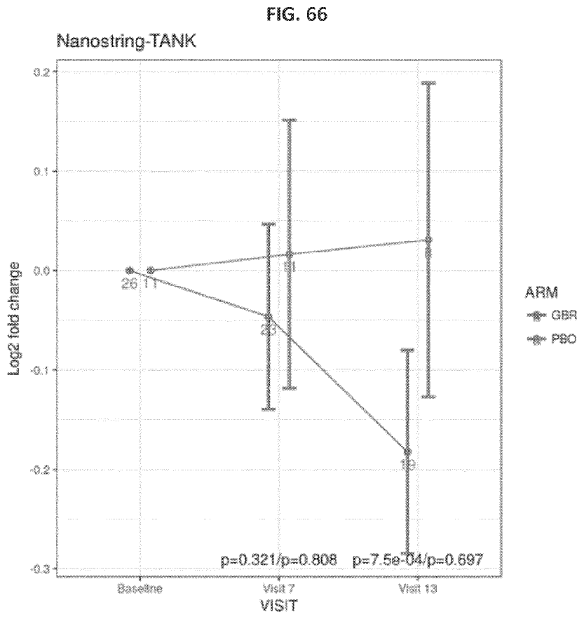

FIG. 66. Least squared means estimates for TANK (NanoString)

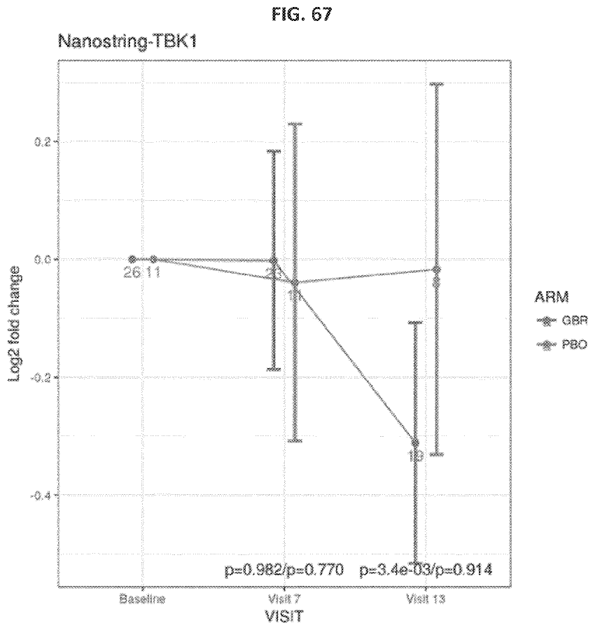

FIG. 67. Least squared means estimates for TBK1 (NanoString)

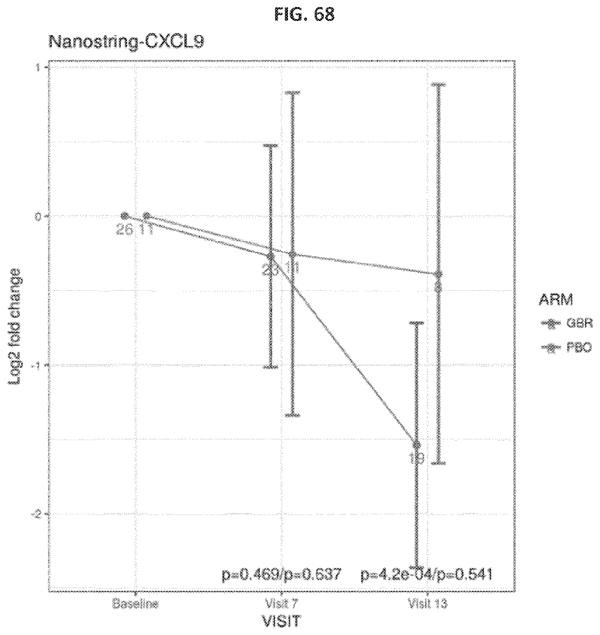

FIG. 68. Least squared means estimates for CXC1.9 (NanoString)

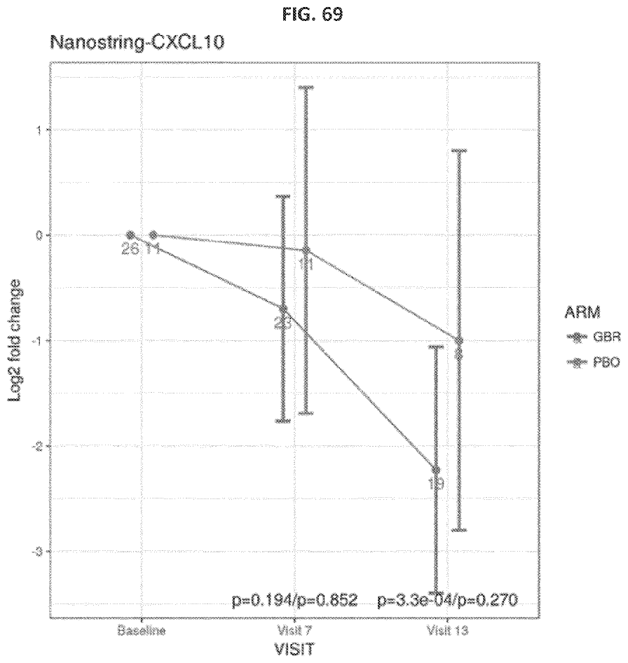

FIG. 69. Least squared means estimates for CXCL10 (NanoString)

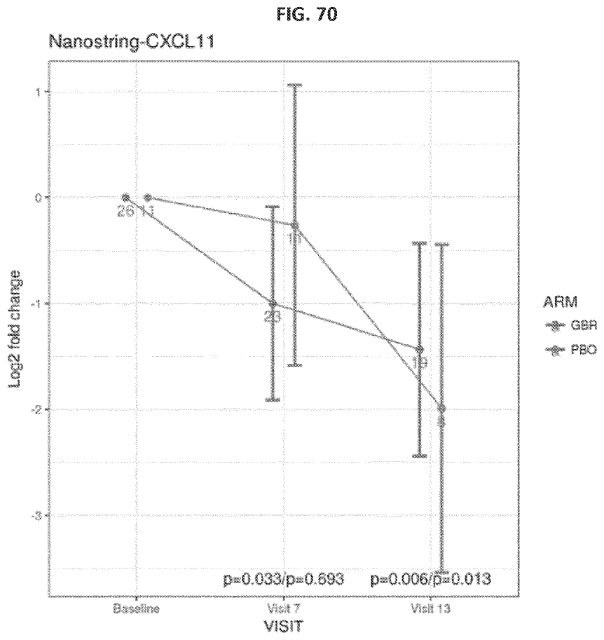

FIG. 70. Least squared means estimates for CXCL11 (NanoString)

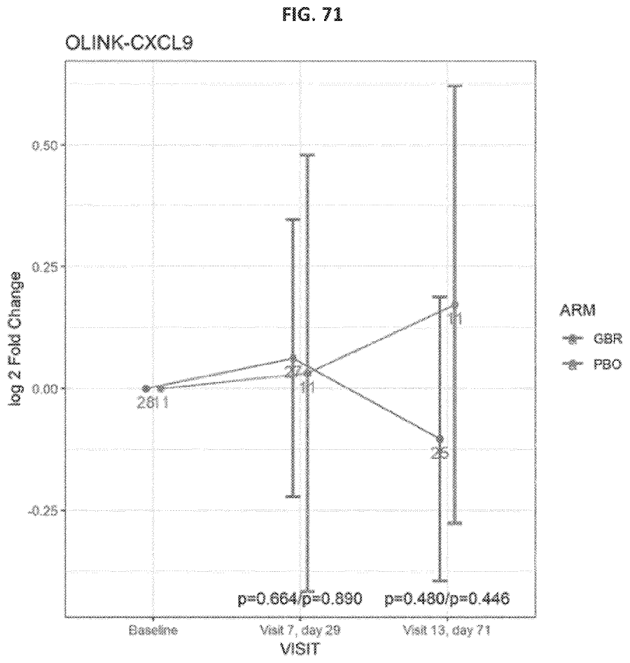

FIG. 71. Least squared means estimates for CXCL9 (OLINK)

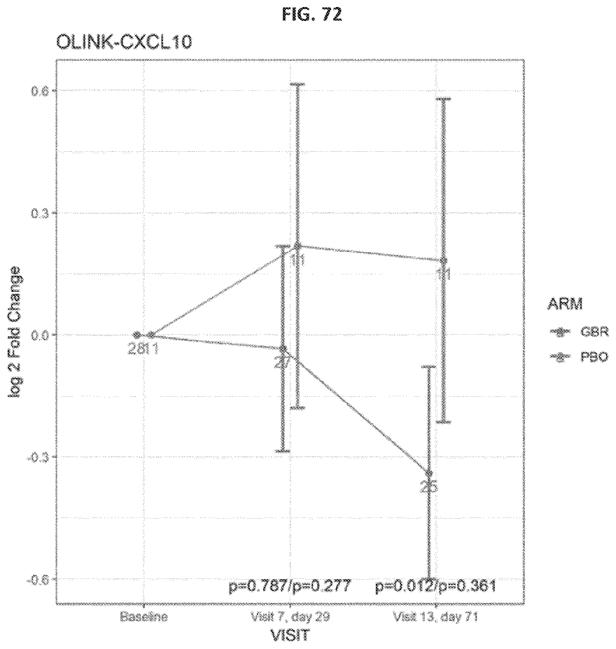

FIG. 72. Least squared means estimates for CXCL10 (OLINK)

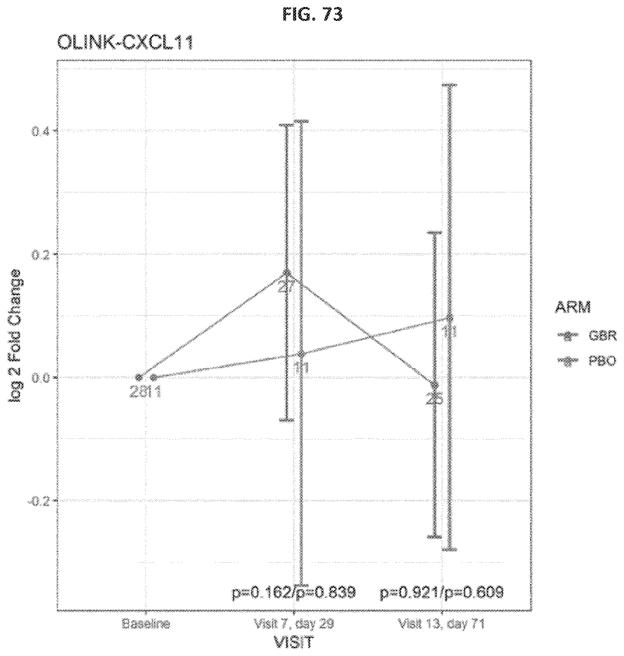

FIG. 73. Least squared means estimates for CXCL11 (OLINK)

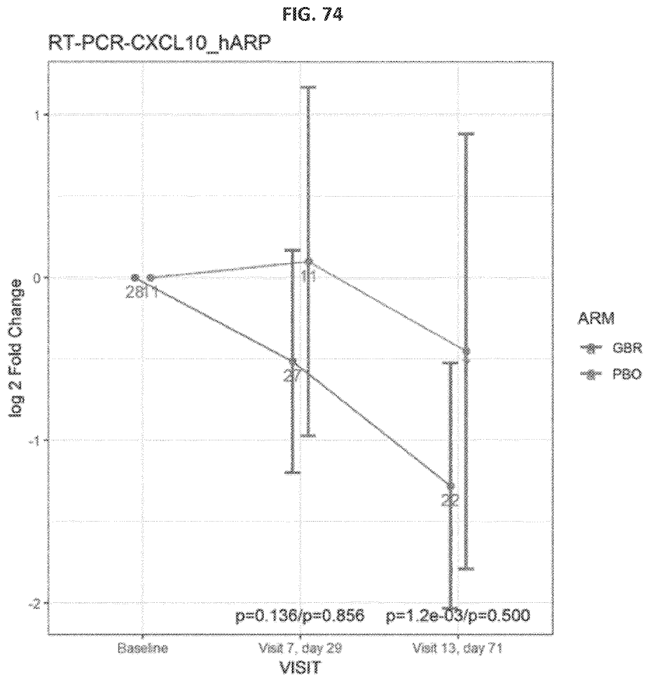

FIG. 74. Least squared means estimates for CXCL10 (RT-PCR)

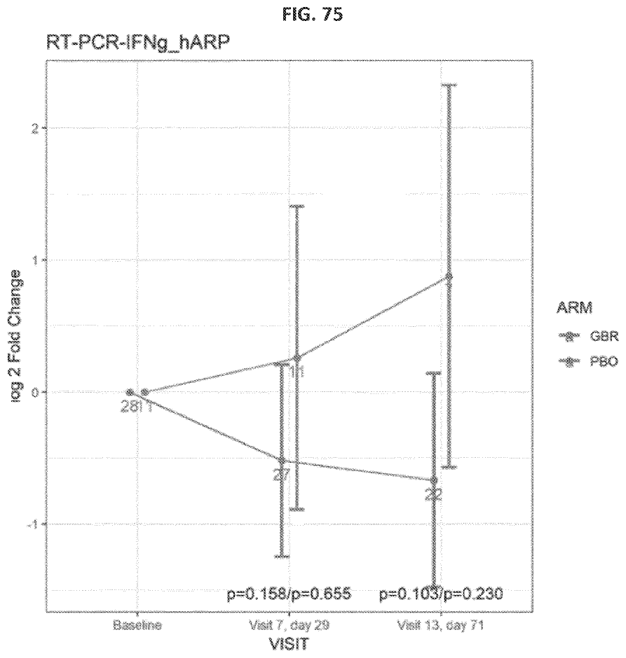

FIG. 75. Least squared means estimates for IFN gamma (RT-PCR)

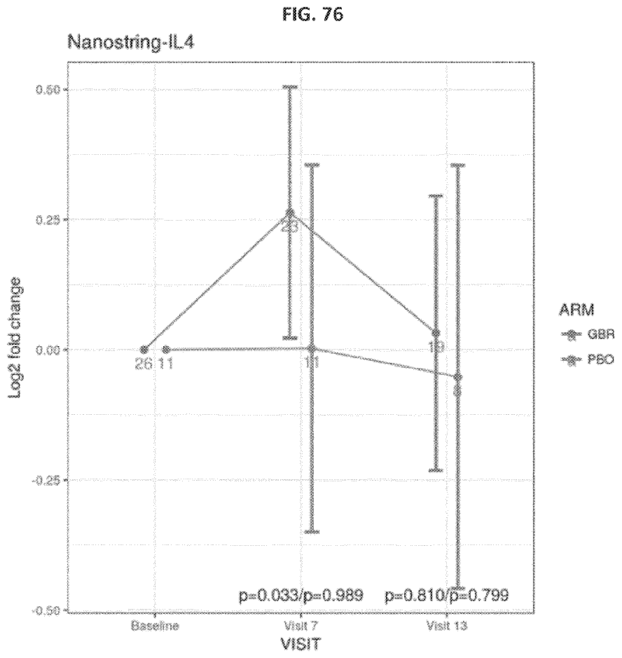

FIG. 76. Least squared means estimates for IL4 (NanoString)

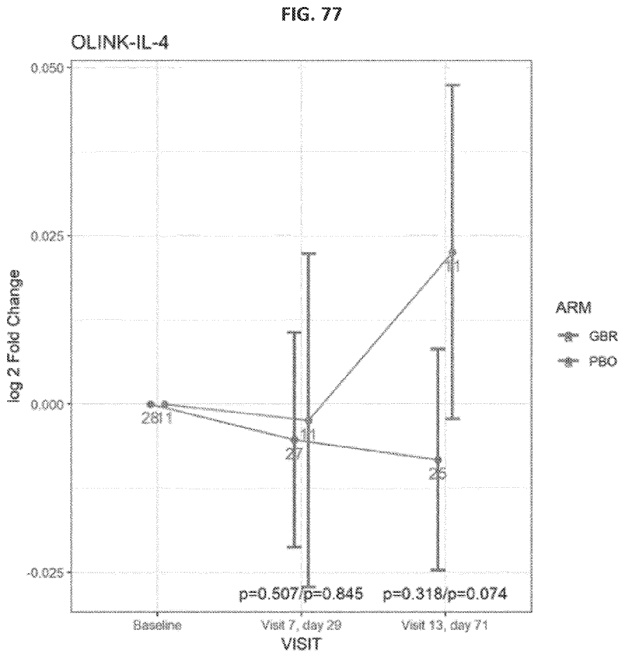

FIG. 77. Least squared means estimates for IL4 (OLINK)

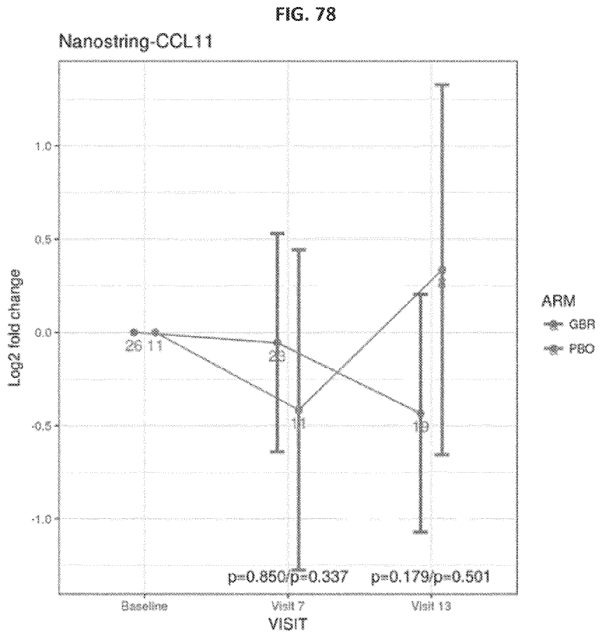

FIG. 78. Least squared means estimates for CCL11 (NanoString)

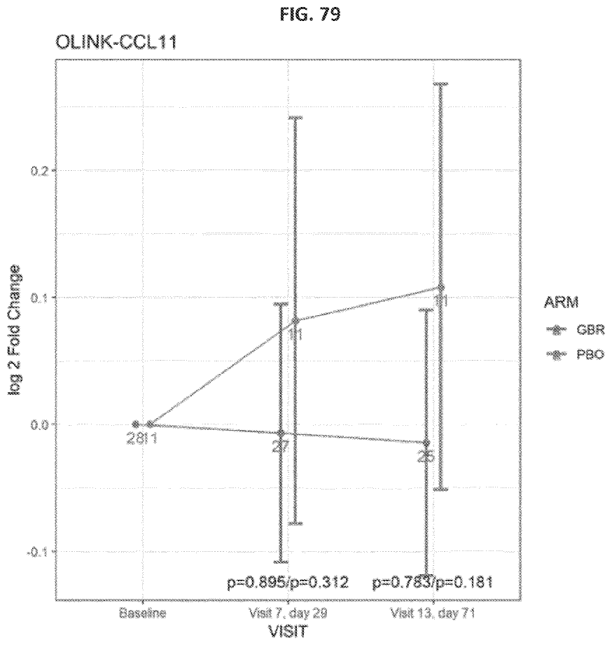

FIG. 79. Least squared means estimates for CCL11 (OLINK)

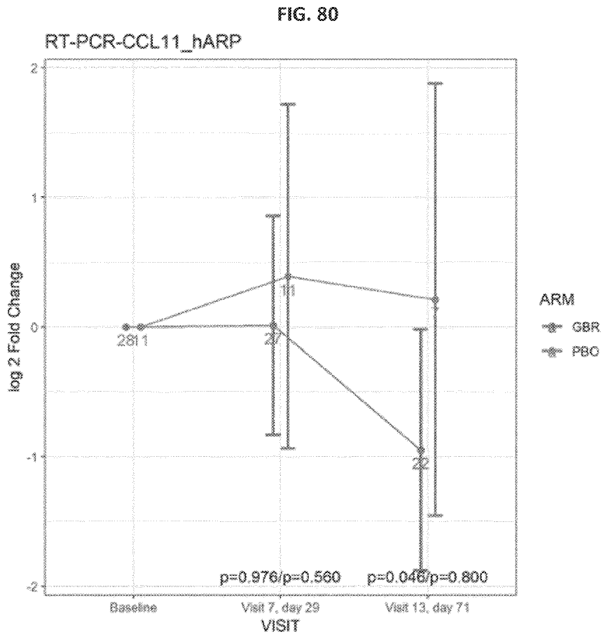

FIG. 80. Least squared means estimates for CCL11 (RT-PCR)

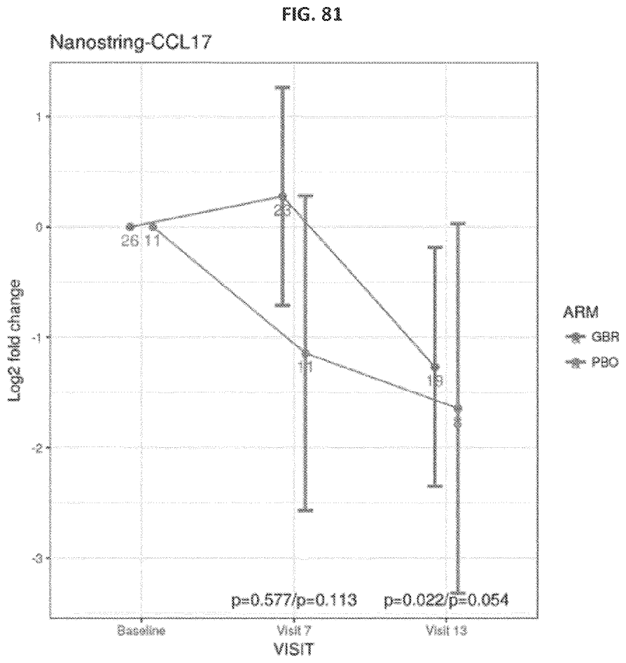

FIG. 81. Least squared means estimates for CCL17 (NanoString)

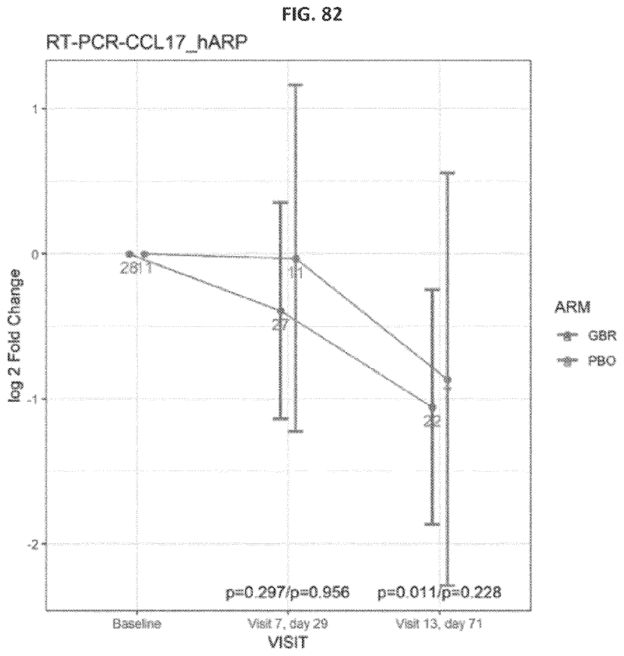

FIG. 82. Least squared means estimates for CCL17 (RT-PCR)

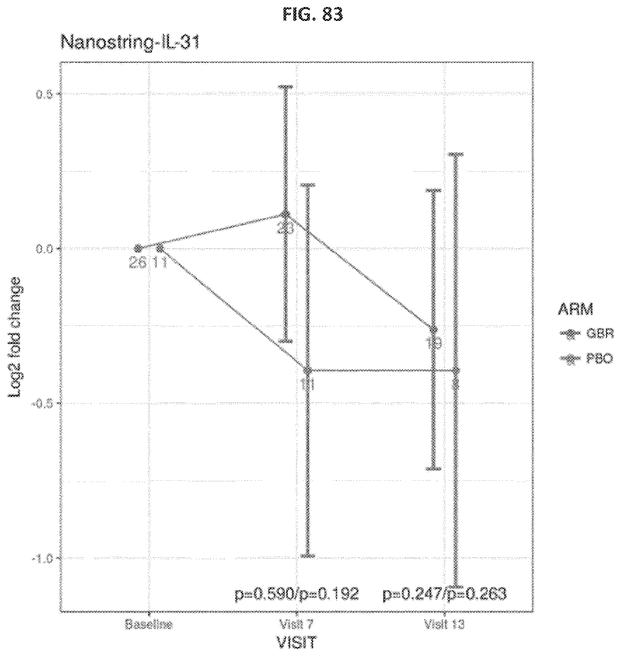

FIG. 83. Least squared means estimates for IL-31 (NanoString)

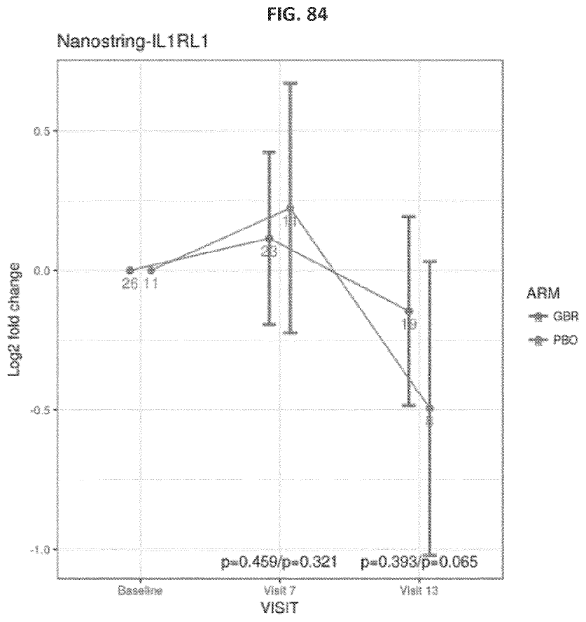

FIG. 84. Least squared means estimates for IL1RL1 (NanoString)

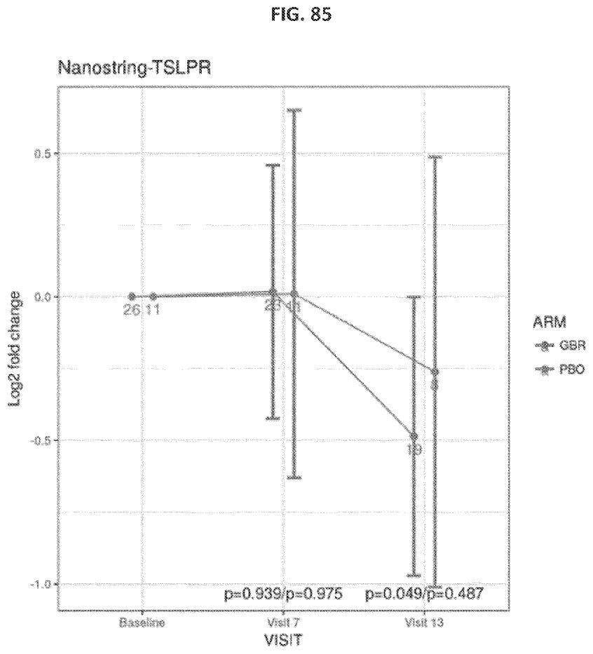

FIG. 85. Least squared means estimates for TSLPR (NanoString)

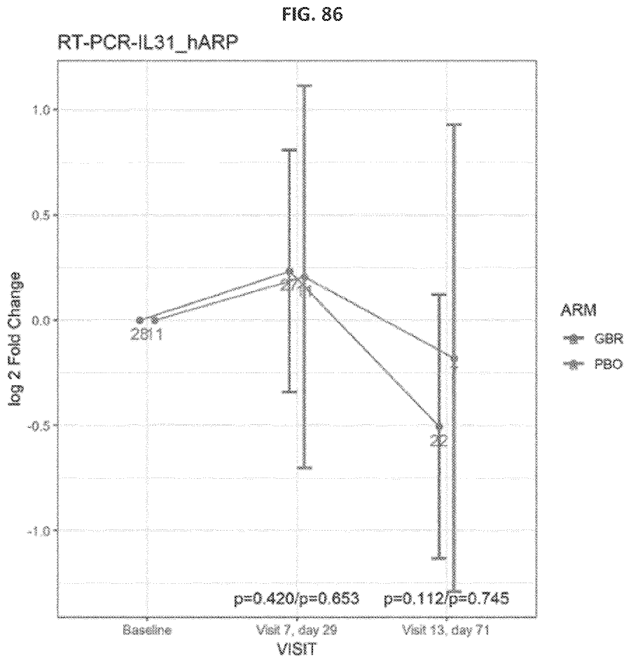

FIG. 86. Least squared means estimates for IL31 (RT-PCR)

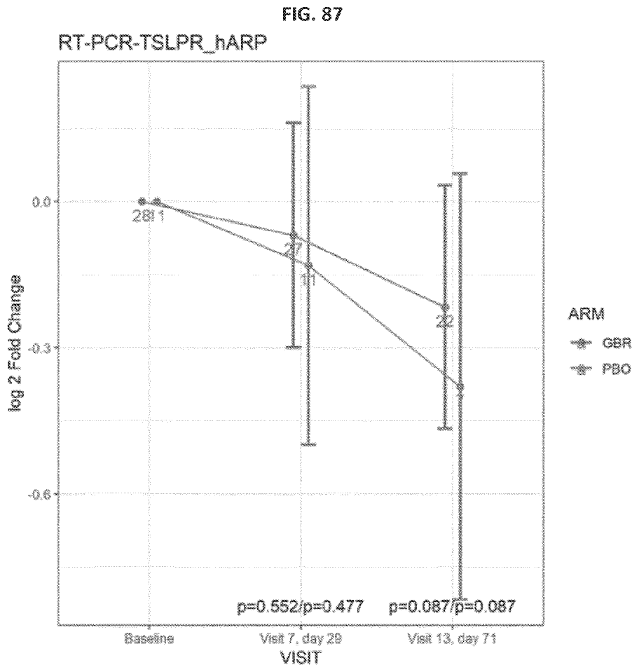

FIG. 87. Least squared means estimates for TSLPR (RT-PCR)

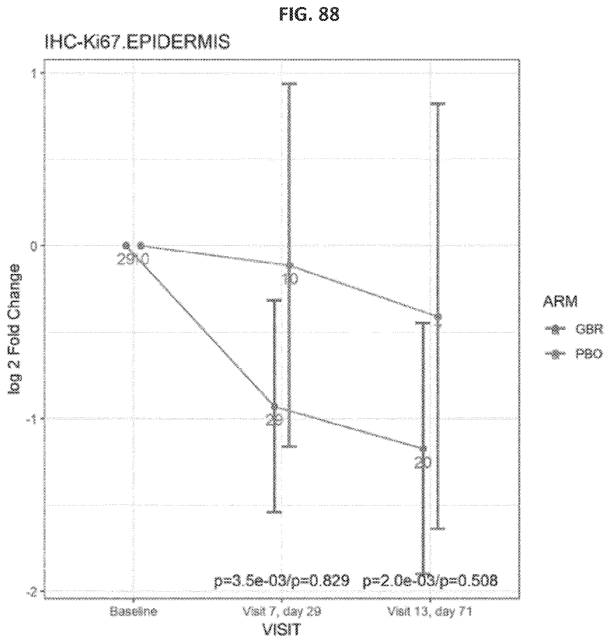

FIG. 88. Least squared means estimates for Ki67

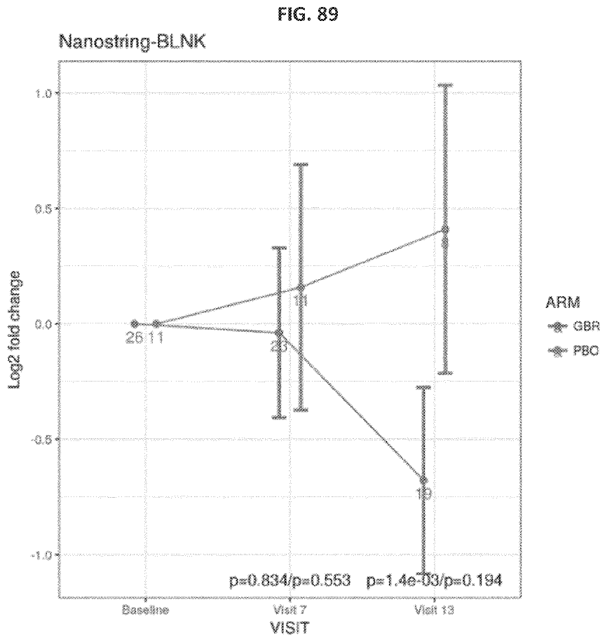

FIG. 89. Least squared means estimates for BLNK (NanoString)

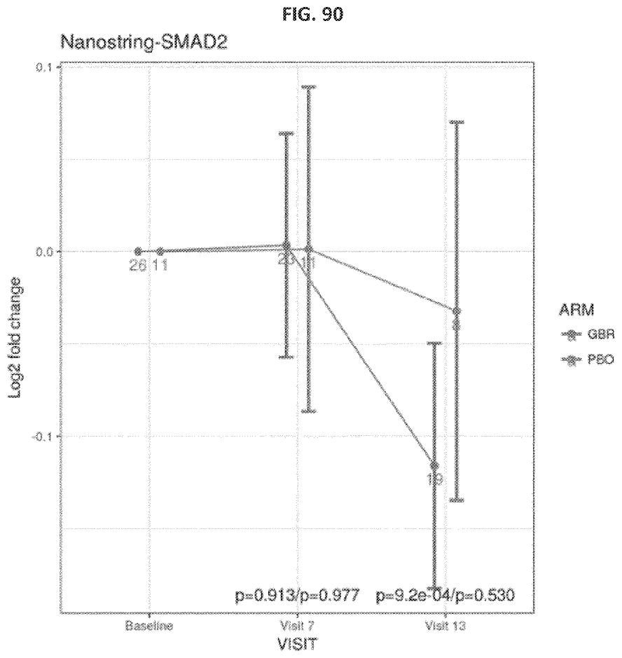

FIG. 90. Least squared means estimates for SMAD2 (NanoString)

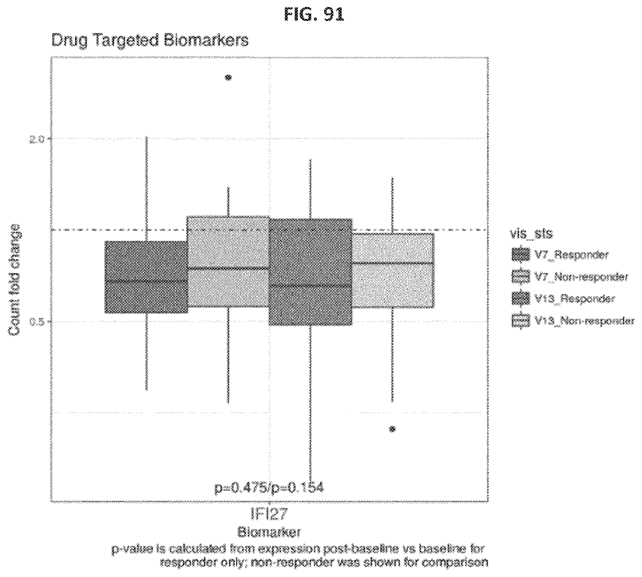

FIG. 91. Boxplot for IFI27 in GBR 830 arm only by response status (NanoString)

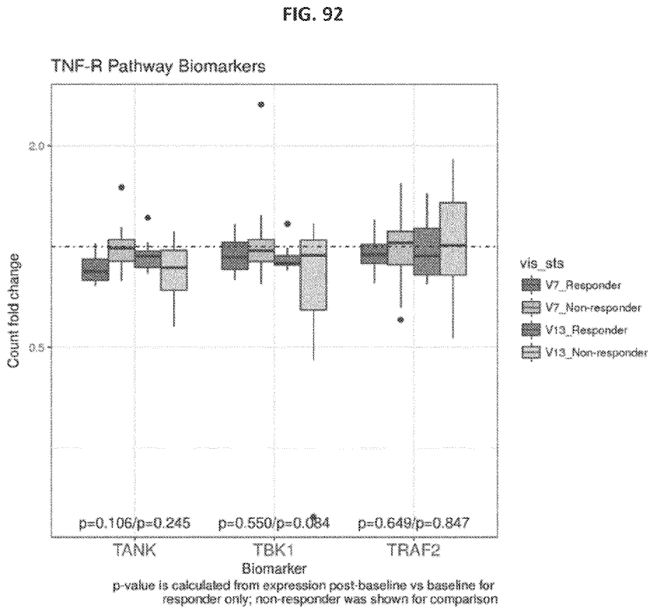

FIG. 92. Boxplot for TNF-R pathway in GBR 830 arm only by response status (NanoString)

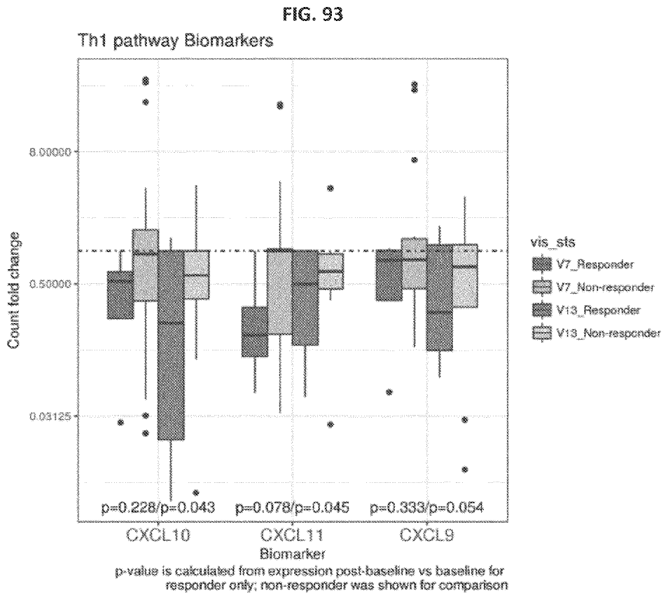

FIG. 93. Boxplot for Th1 pathway in GBR 830 arm only by response status (NanoString)

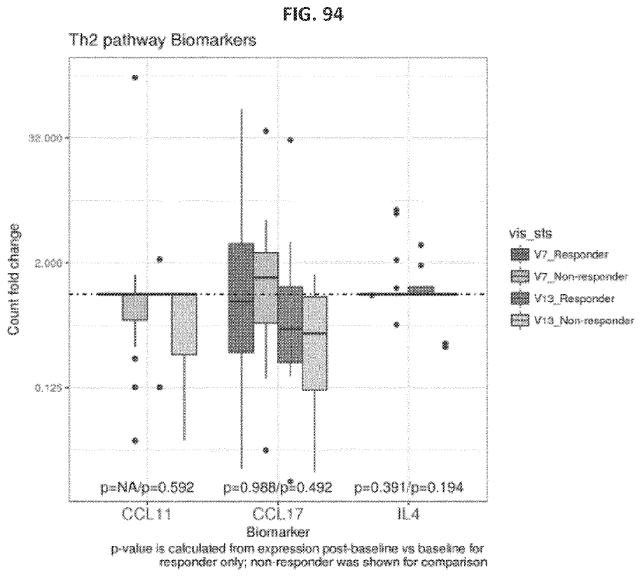

FIG. 94. Boxplot for Th2 pathway in GBR 830 arm only by response status (NanoString)

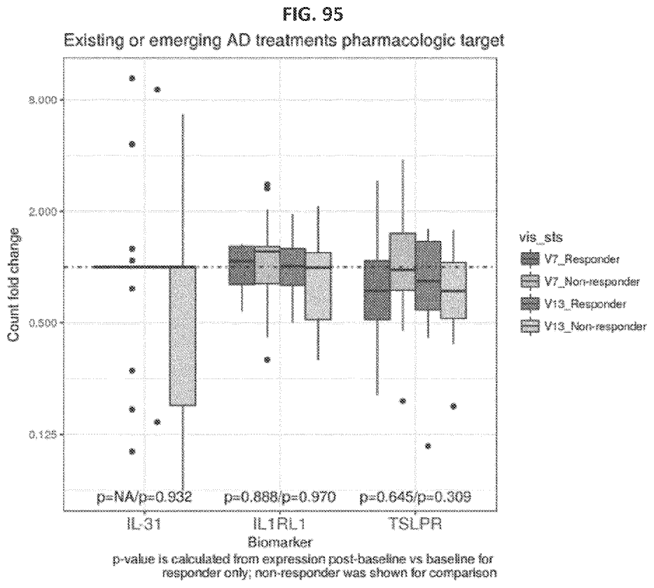

FIG. 95. Boxplot for existing or emerging AD treatments pharmacologic target in GBR 830 arm only by response status (NanoString)

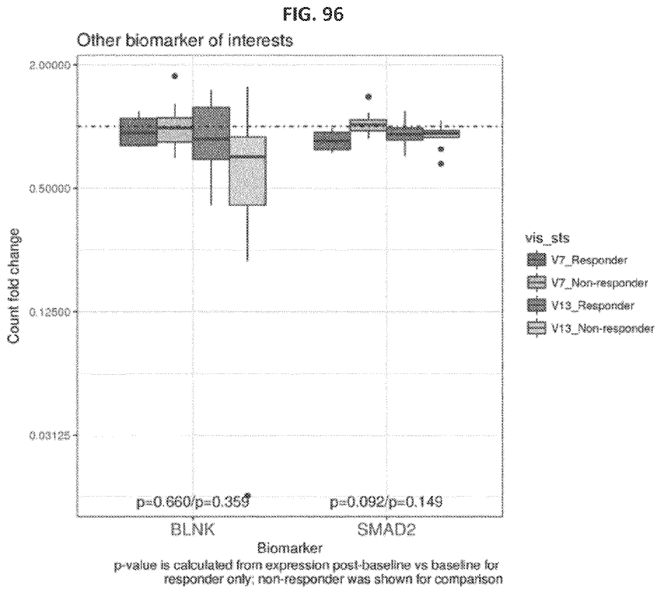

FIG. 96. Boxplot for other genes of interest in GBR 830 arm only by response status (NanoString)

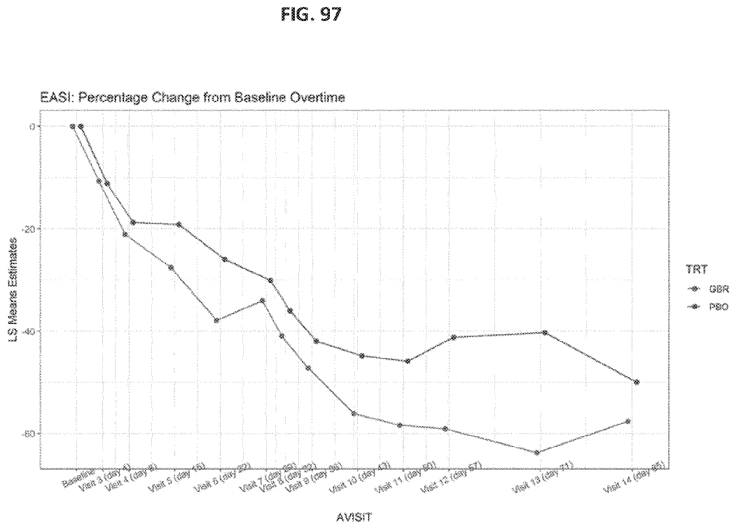

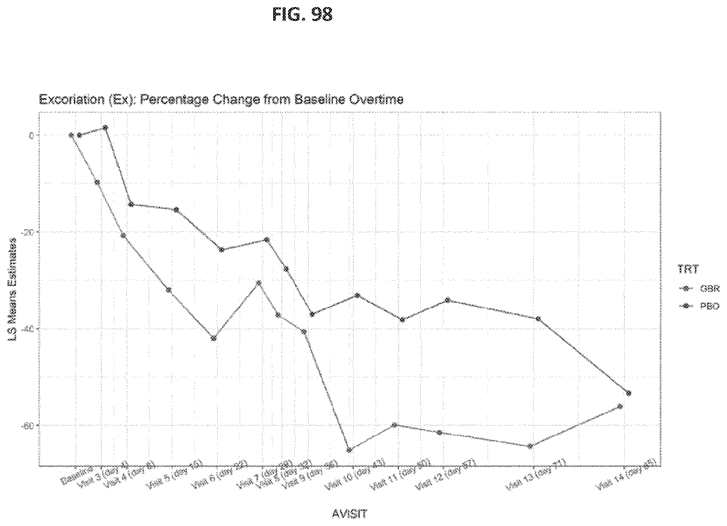

FIG. 97. EASI score change from baseline over time

FIG. 98 Excoriation score change from baseline over time

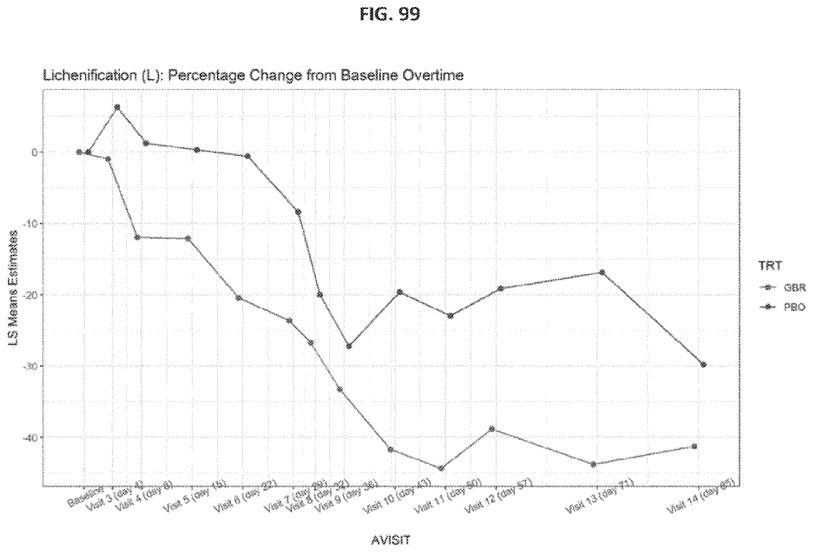

FIG. 99. Lichenification score change from baseline over time

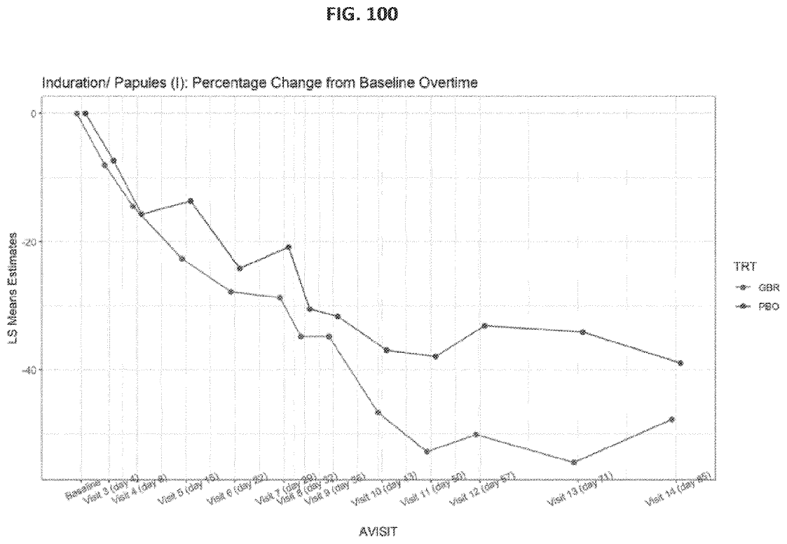

FIG. 100. Induration score change from baseline over time

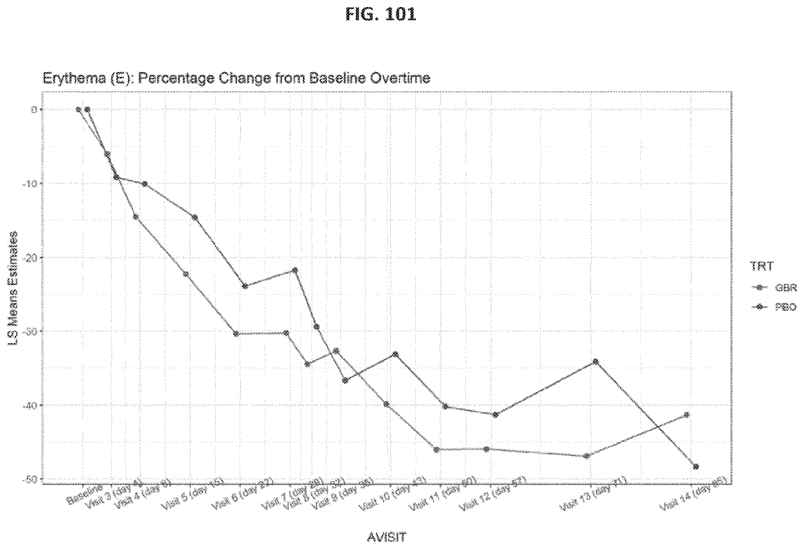

FIG. 101. Erythema score change from baseline over time

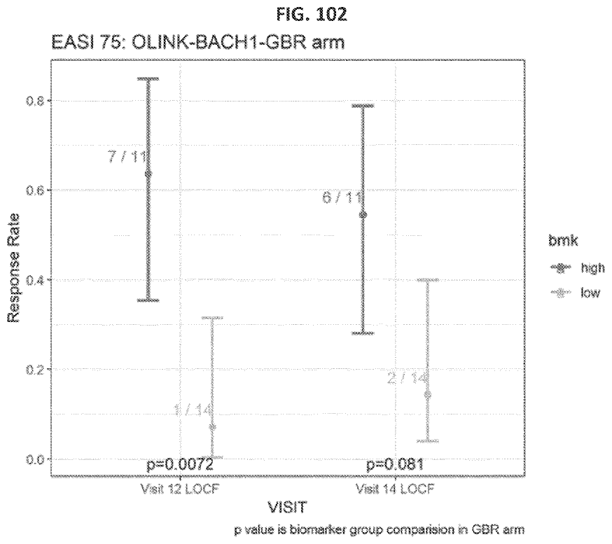

FIG. 102. Response estimates for BACH1 (OLINK)

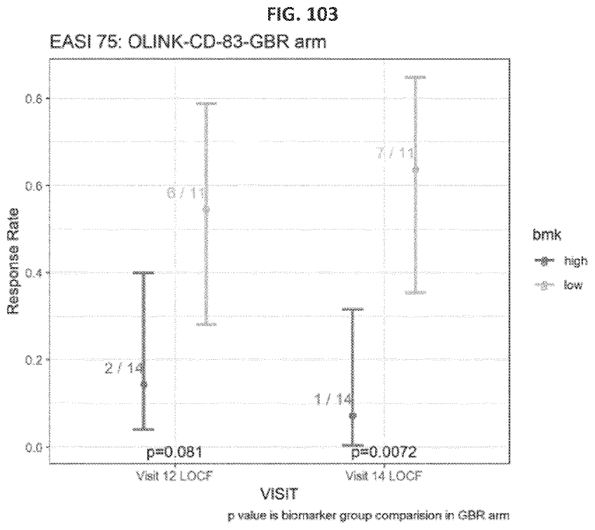

FIG. 103. Response estimates for CD-83 (OLINK)

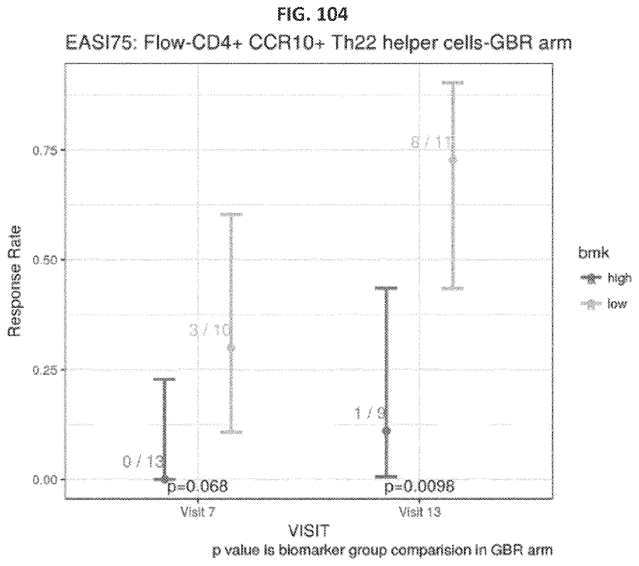

FIG. 104. Response estimate for CD4+CCR10+Th22 helper cells

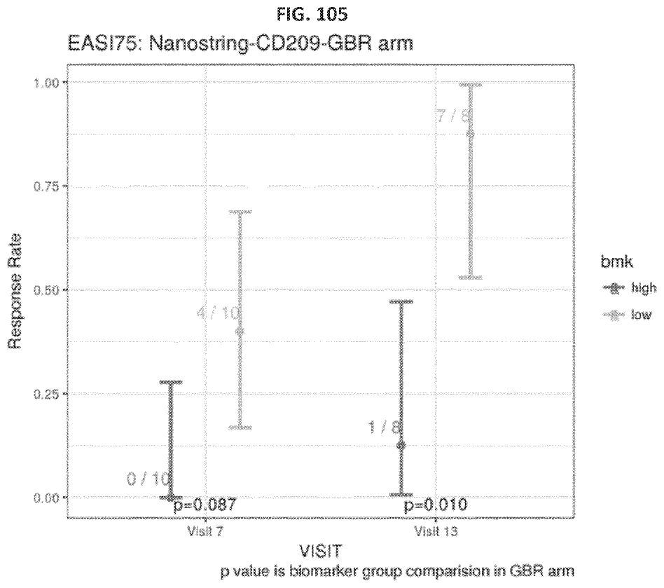

FIG. 105. Response estimates for CD209 (NanoString)

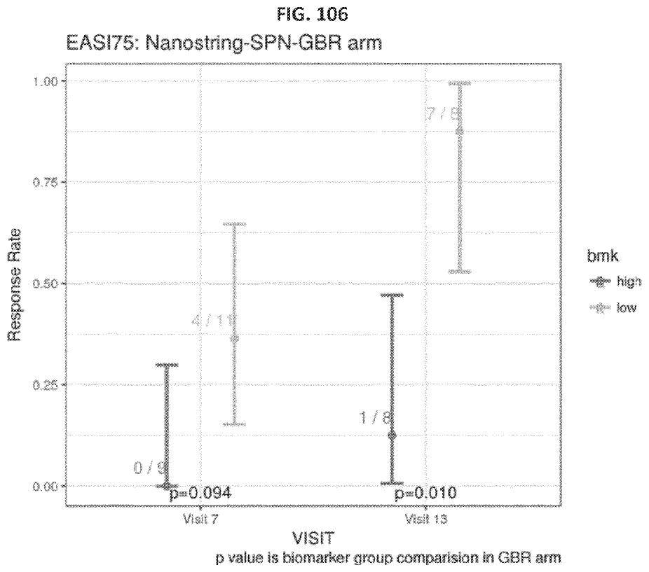

FIG. 106. Response estimates for SPN (NanoString)

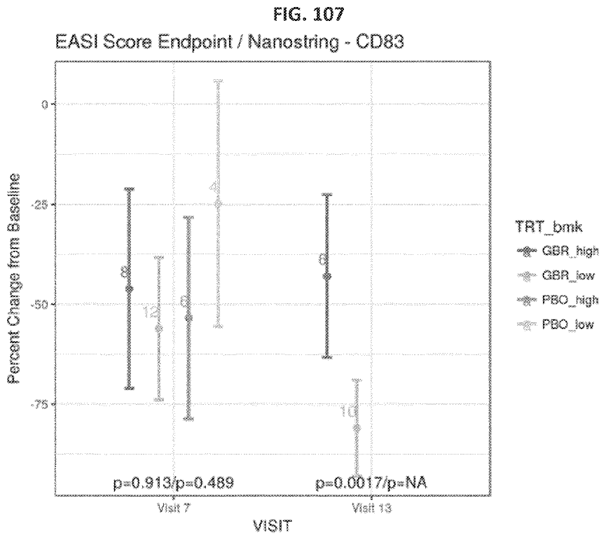

FIG. 107 Least squared means estimates for CD83 (NanoString)

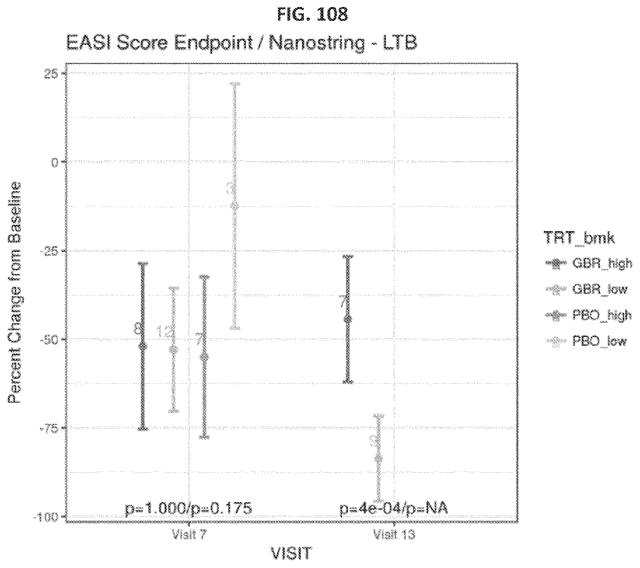

FIG. 108. Least squared means estimates for LTB (NanoString)

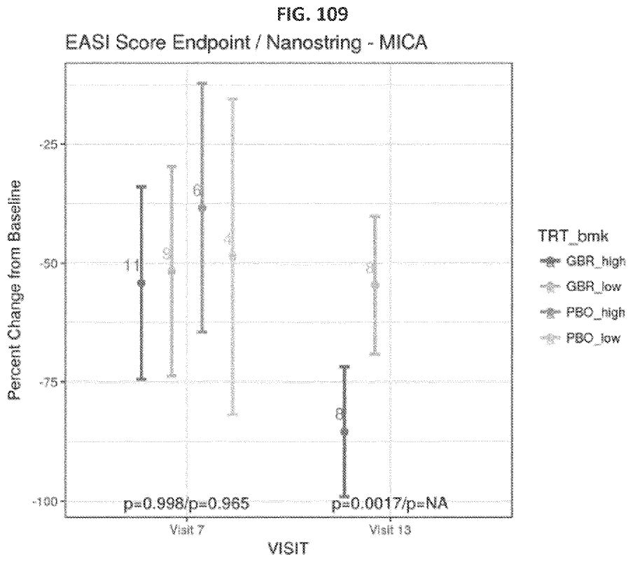

FIG. 109. Least squared means estimates for MICA (NanoString)

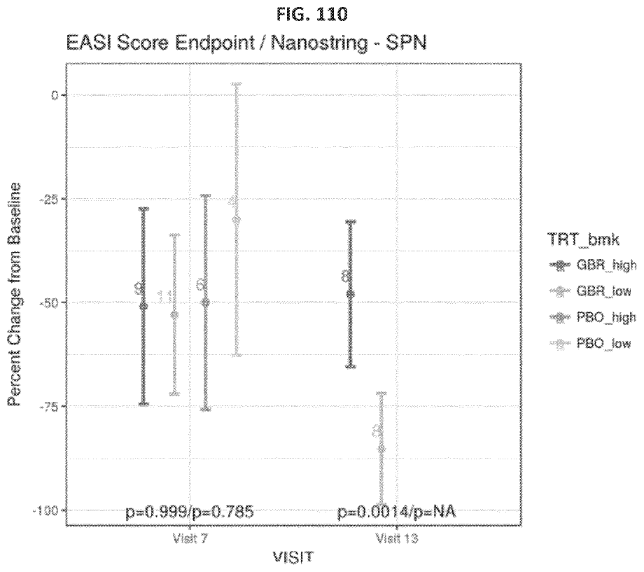

FIG. 110. Least squared means estimates for SPN (NanoString)

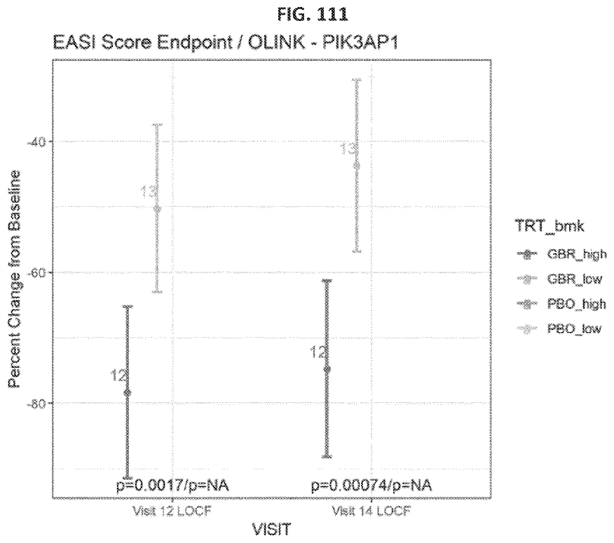

FIG. 111. Least squared means estimates for PIK3AP1 (OLINK)

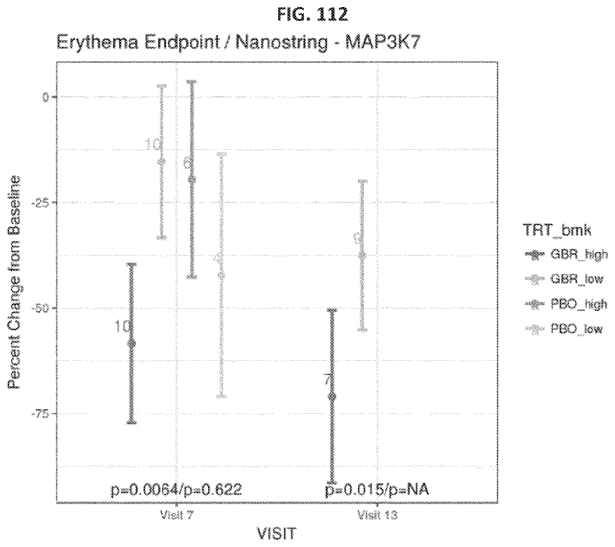

FIG. 112. Least squared means estimates for MAP3K7 (NanoString)

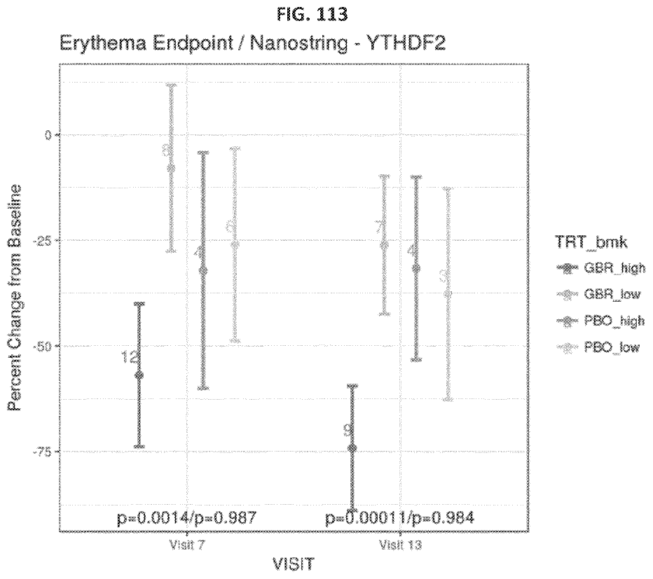

FIG. 113. Least squared means estimates for YTHDF2 (NanoString)

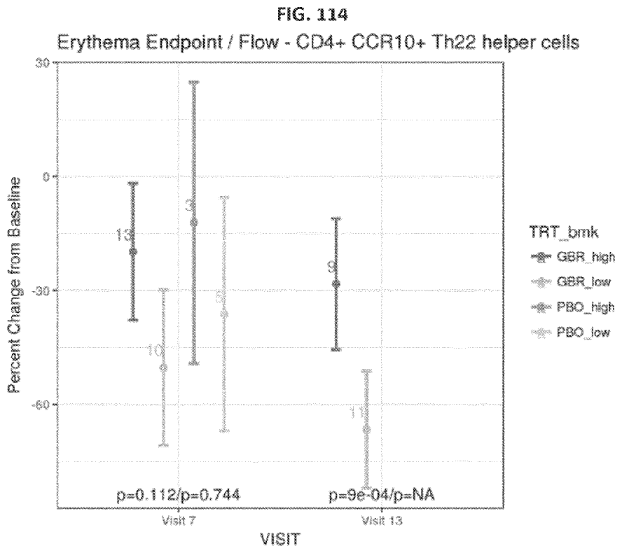

FIG. 114. Least squared means estimates for CD4+CCR10+Th22 helper cells (Flow)

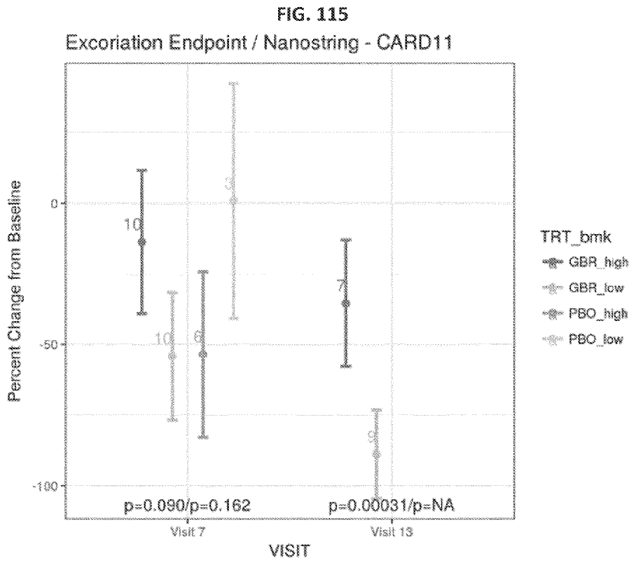

FIG. 115. Least squared means estimates for CARD11 (NanoString)

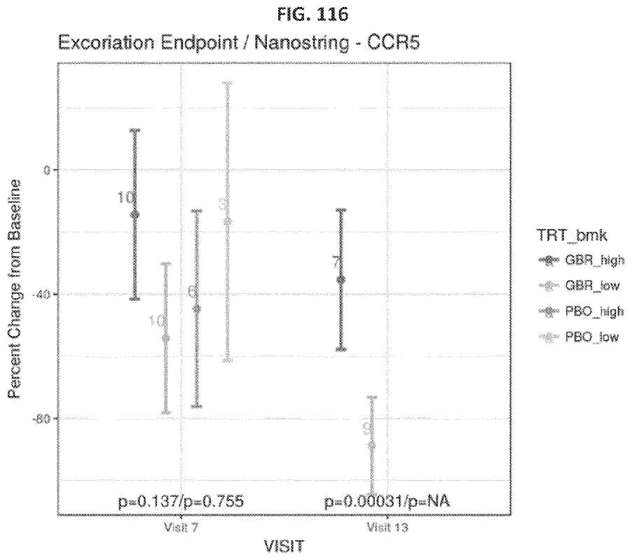

FIG. 116. Least squared means estimates for CCR5 (NanoString)

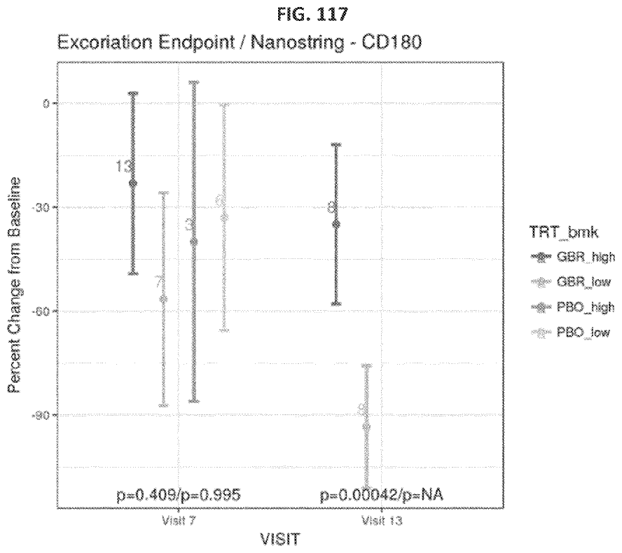

FIG. 117. Least squared means estimates for CD180 (NanoString)

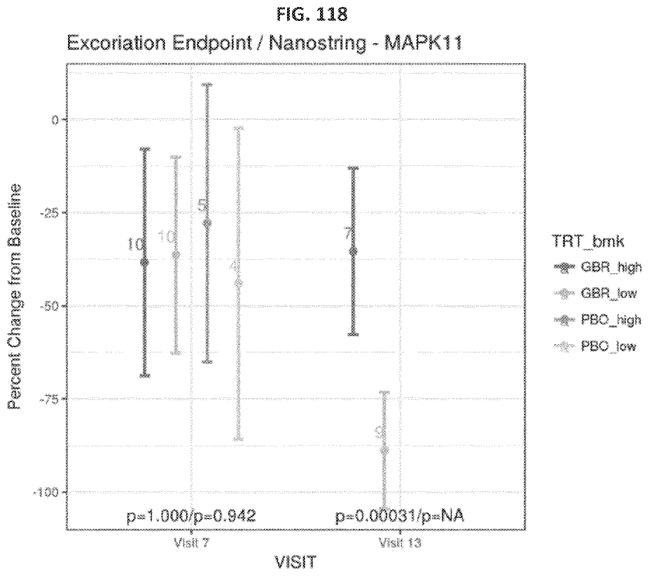

FIG. 118. Least squared means estimates for MAPK11 (NanoString

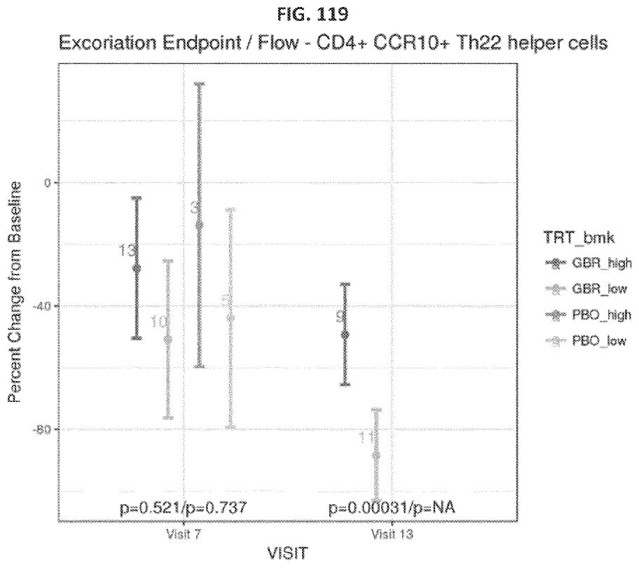

FIG. 119. Least squared means estimates for CD4+CCR10+Th22 helper cells (Flow)

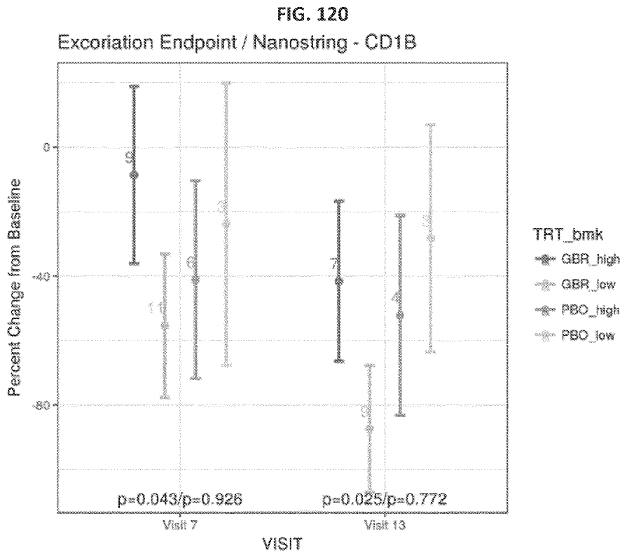

FIG. 120. Least squared means estimates for CD1B (NanoString)

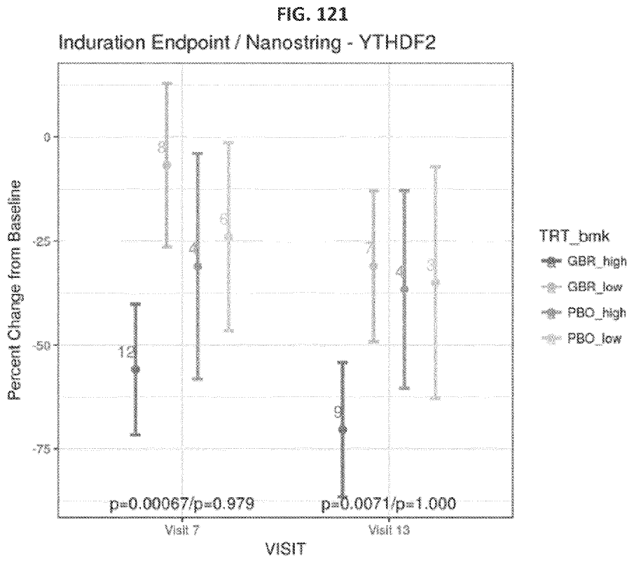

FIG. 121. Least squared means estimates for YTHDF2 (NanoString)

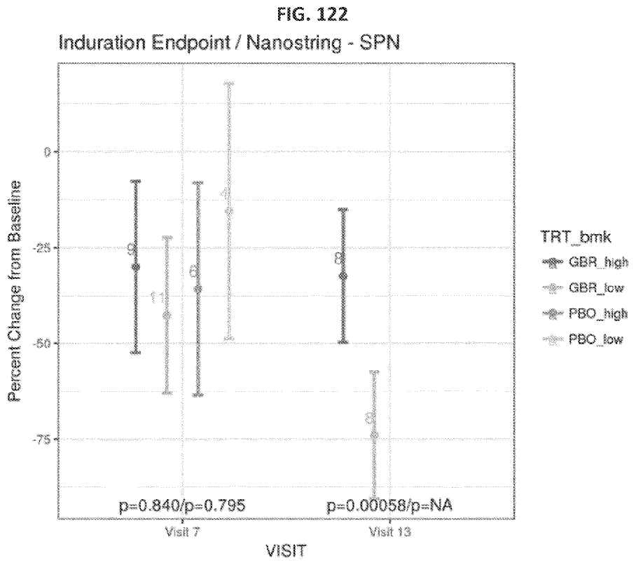

FIG. 122. Least squared means estimates for SPN (NanoString)

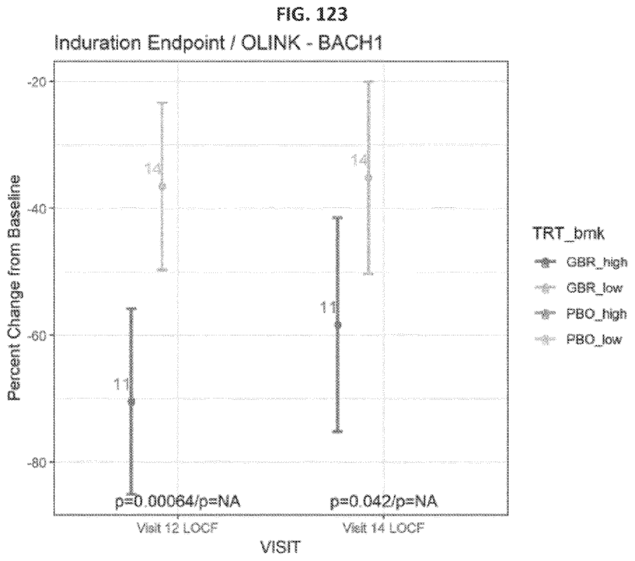

FIG. 123 Least squared means estimates for BACH1 (OLINK)

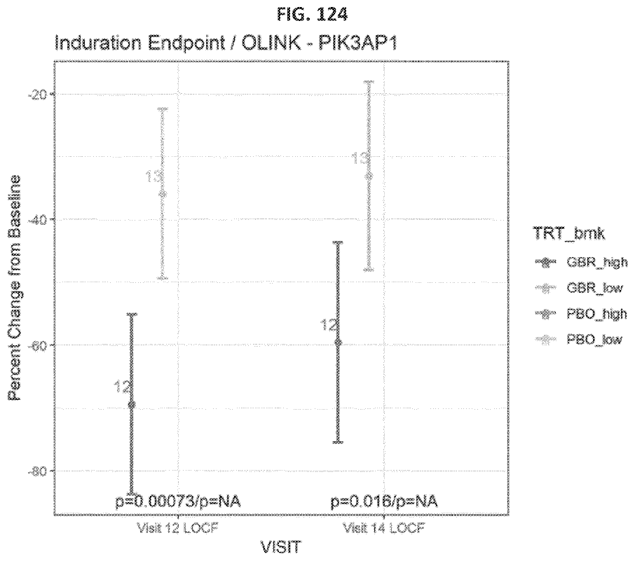

FIG. 124 Least squared means estimates for PIK3AP1 (OLINK)

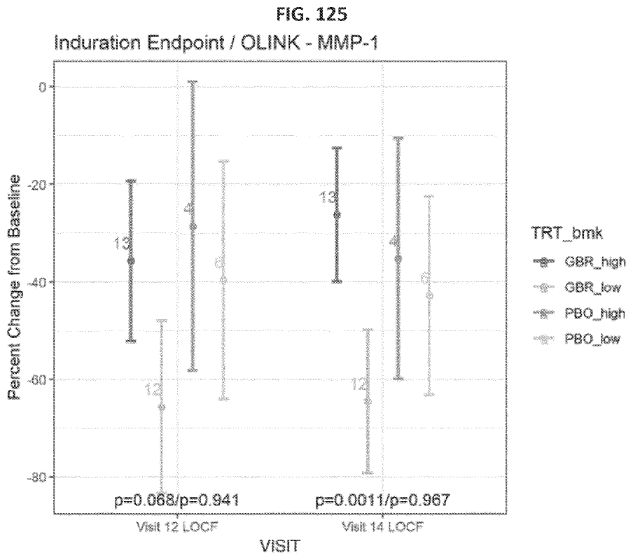

FIG. 125 Least squared means estimates for MMP-1 (OLINK)

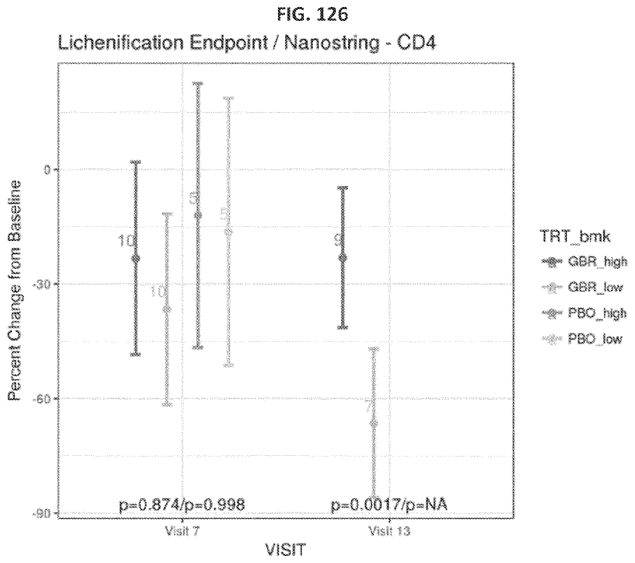

FIG. 126 Least squared means estimates for CD4 (NanoString)

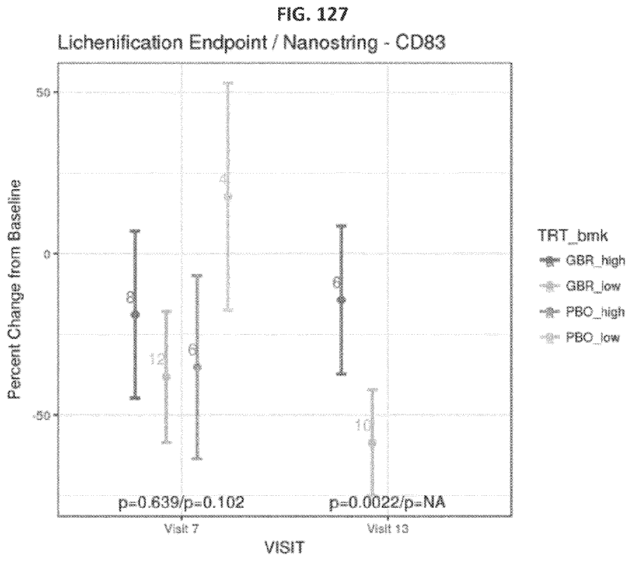

FIG. 127 Least squared means estimates for CD83 (NanoString)

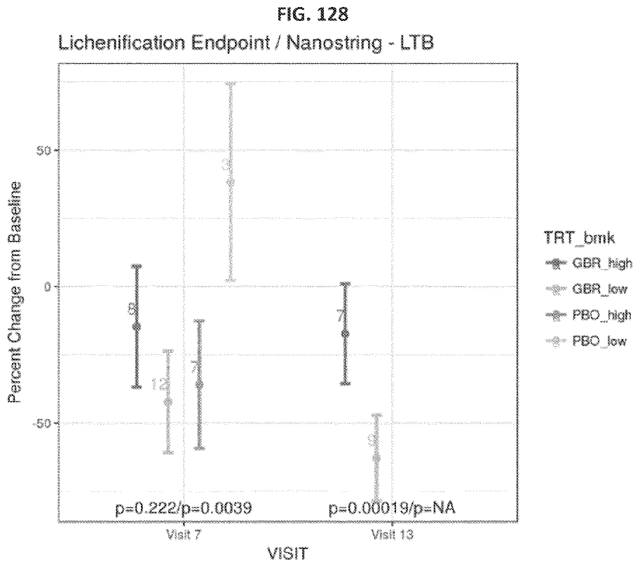

FIG. 128 Least squared means estimates for LTB (NanoString)

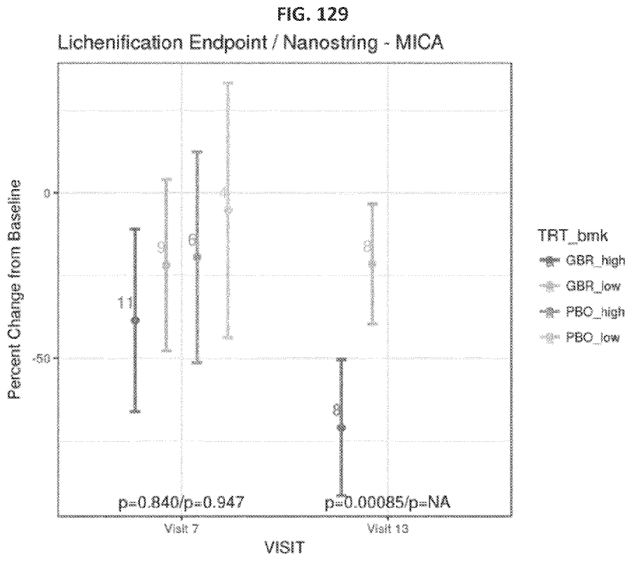

FIG. 129 Least squared means estimates for MICA (NanoString)

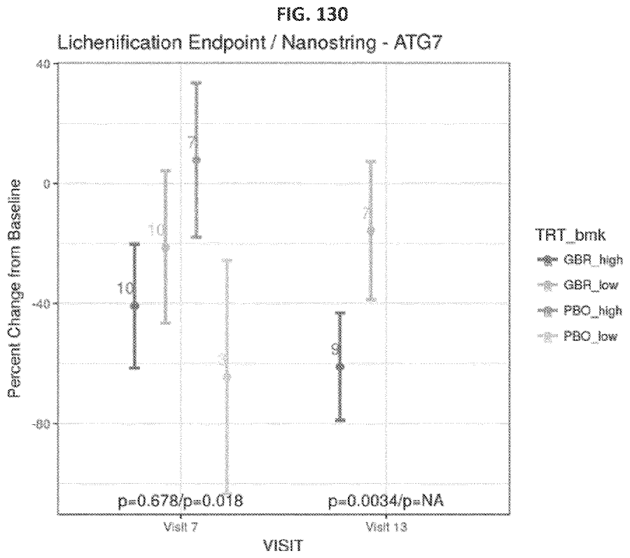

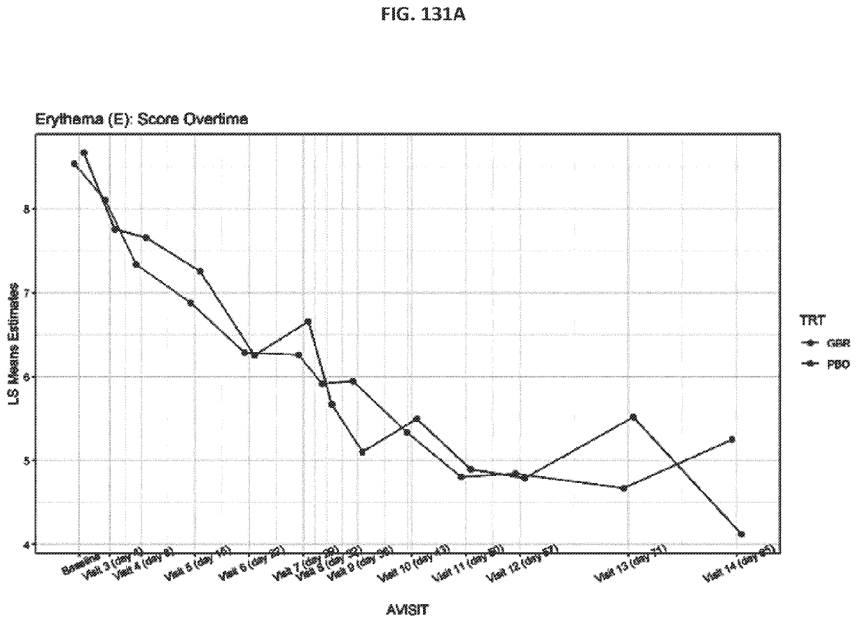

FIG. 130 Least squared means estimates for ATG7 (NanoString)

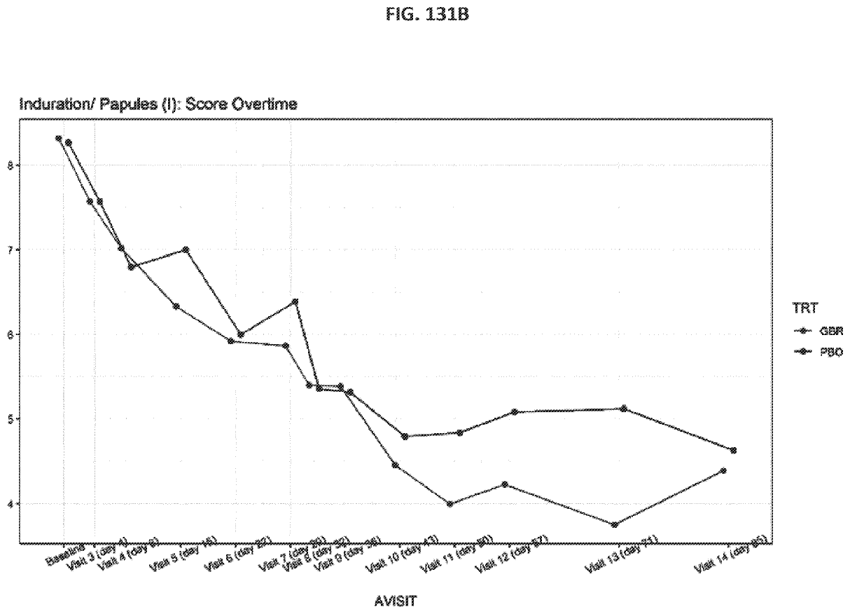

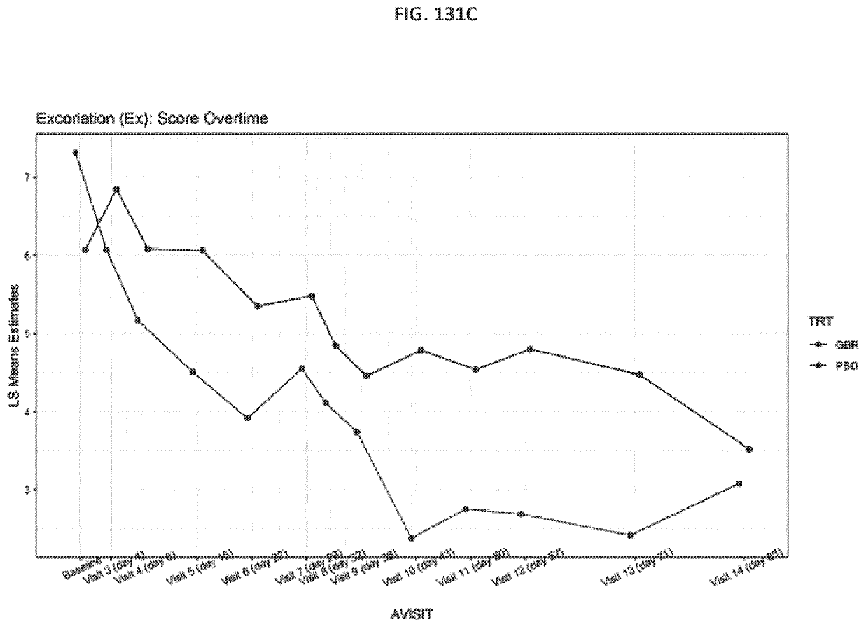

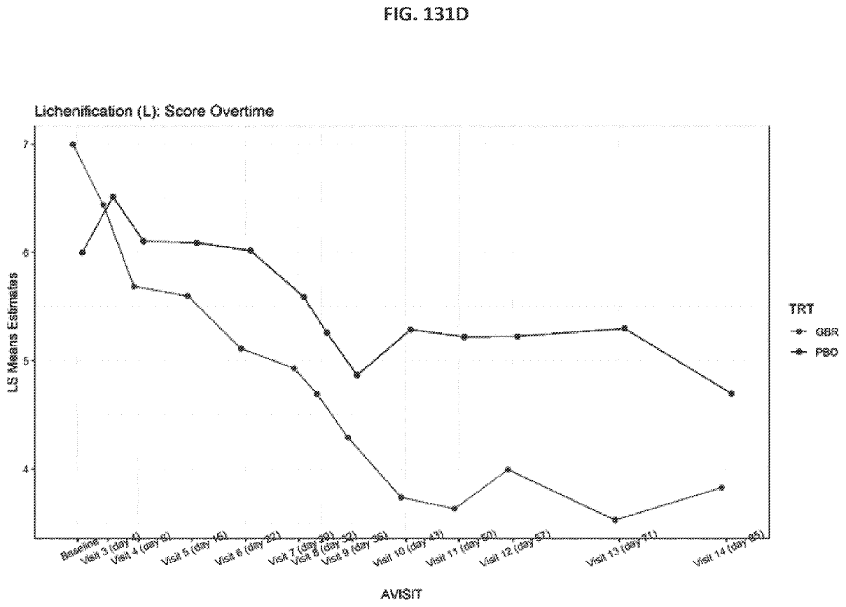

FIG. 131 Additional erythema, induration/papules, excoriation and lichenification results. Top-left panel: Erythema score change from baseline overtime; top-right panel: Induration score change from baseline overtime; bottom-left panel: excoriation score change from baseline overtime; bottom-right panel: Lichenification score change from baseline overtime; bottom-left panel: excoriation score change from baseline overtime

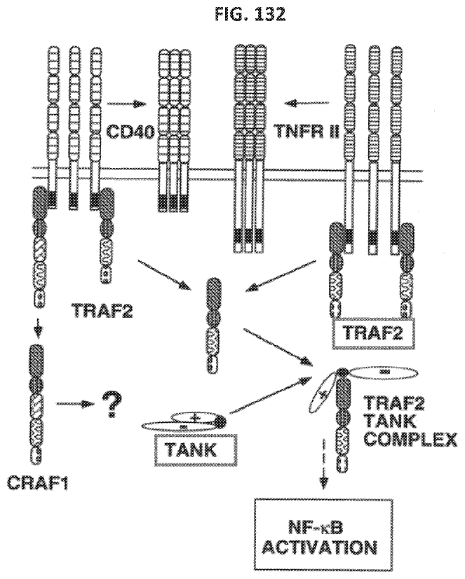

FIG. 132 Schematic pathway

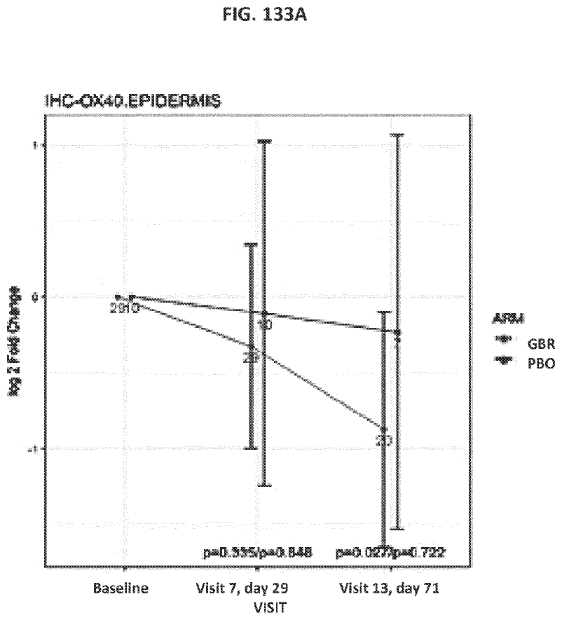

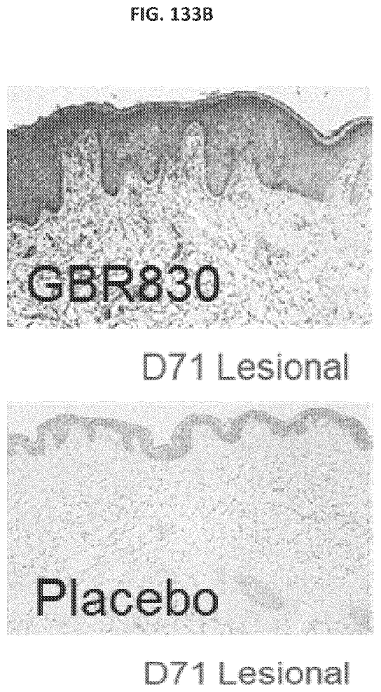

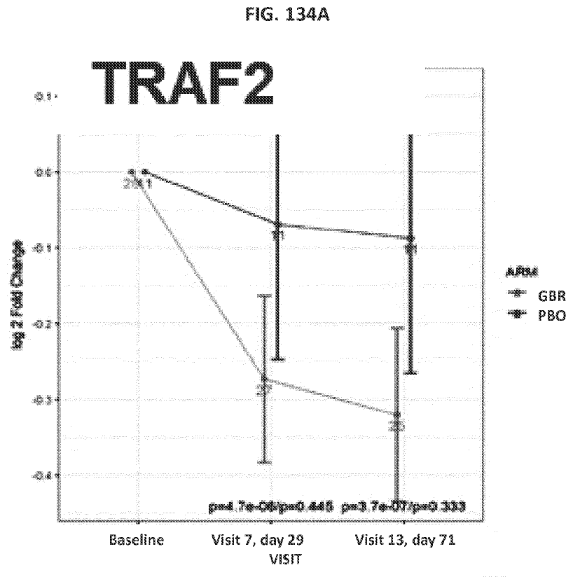

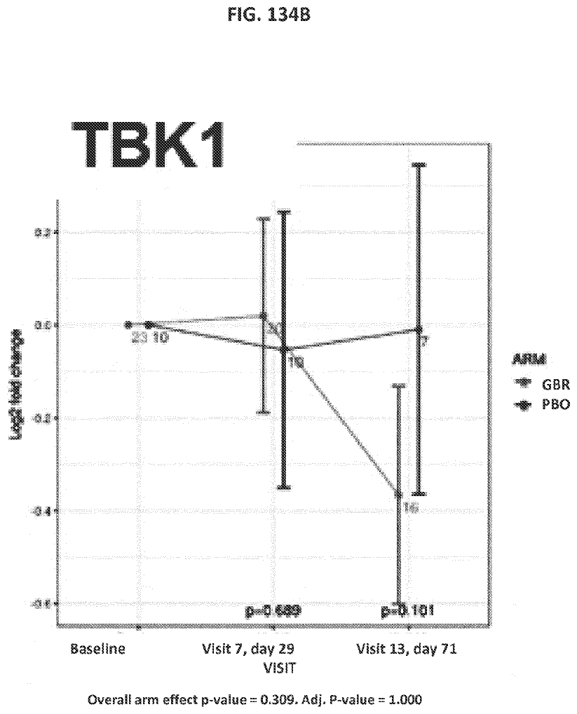

FIG. 133 IHC-OX40 epidermis. Quantification of OX40 in drug treated and placebo treated patients at day 29 and day 71.

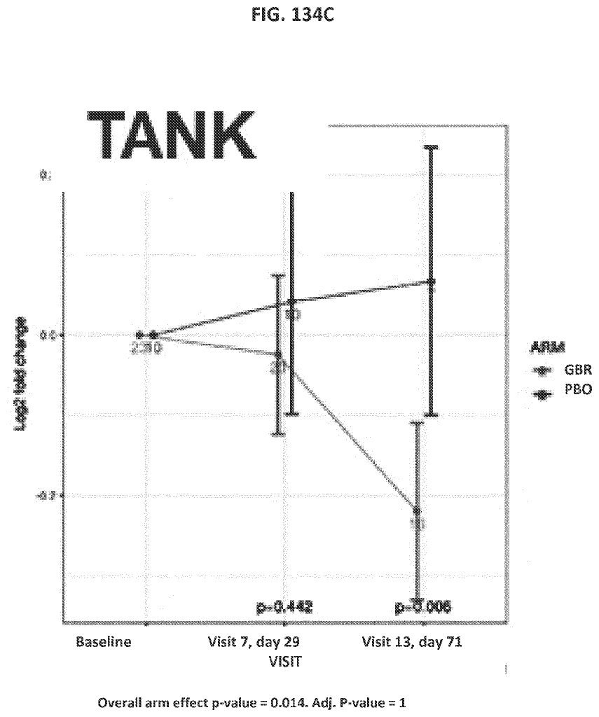

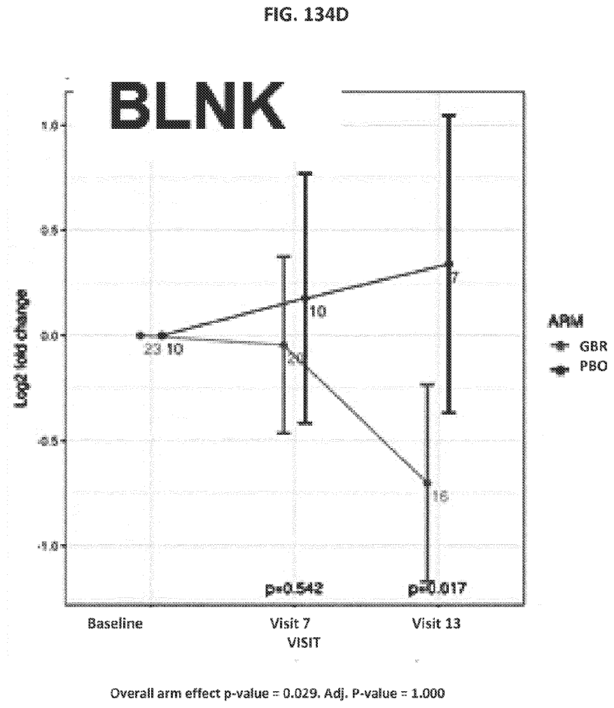

FIG. 134 TRAF2-TBK1-TANK-BLNK. Reduction of OX40 pathway components by GBR830.

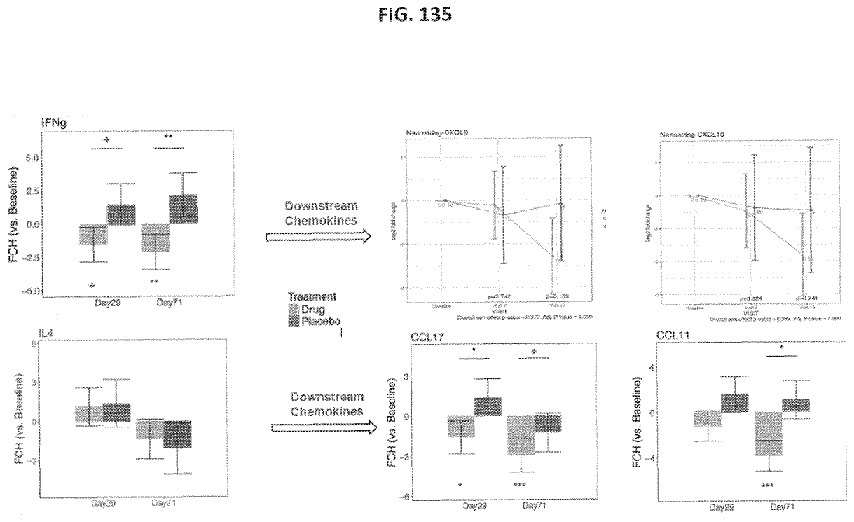

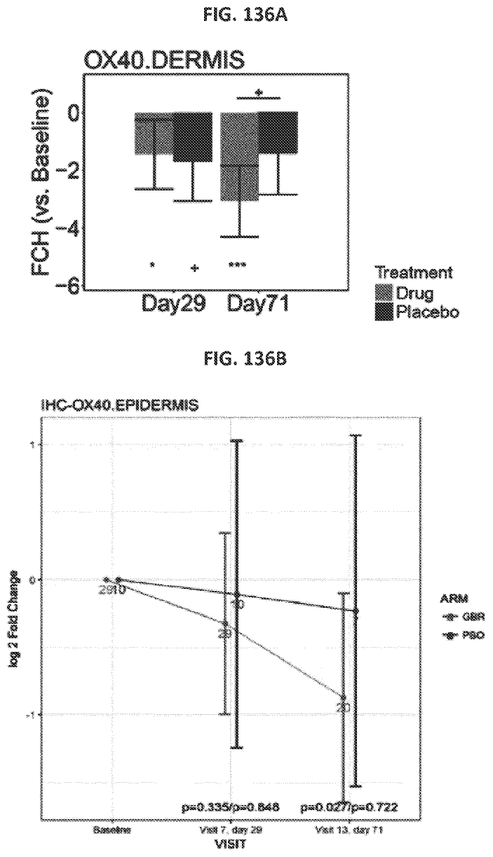

FIG. 135 Measurements of IFNg, CXCL9, CXCL10 (top panels) and of IL4, CCL17 and CCL11, for drug and placebo treated patients at day 29 and Day 71.

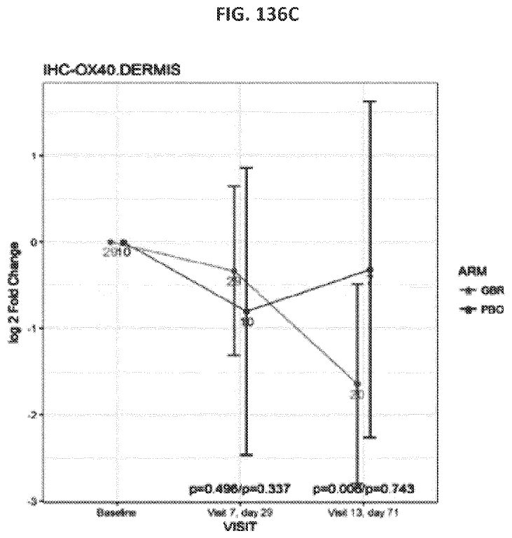

FIG. 136 OX40 reduction in dermis (left and right panels) and in epidermis (middle panel) measured in drug and placebo treated patients at day 29 and Day 71.

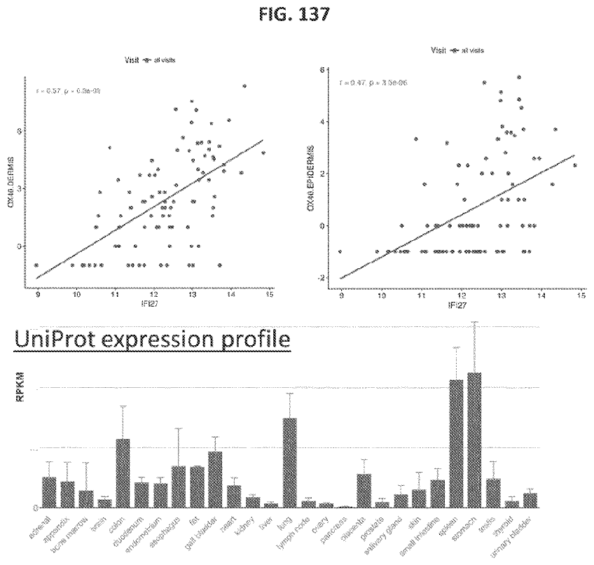

FIG. 137 Correlation between IFI27 and OX40 expression in dermis and epidermis.

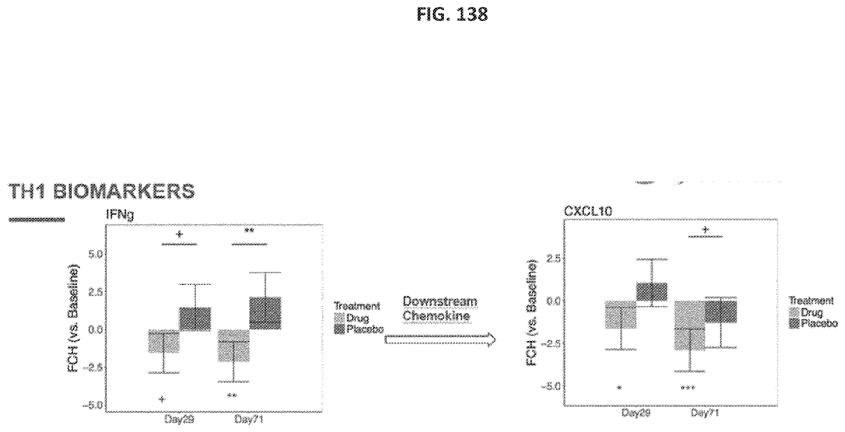

FIG. 138 Measurements of IFNg and CXCL10 for drug and placebo treated patients at day 29 and Day 71.

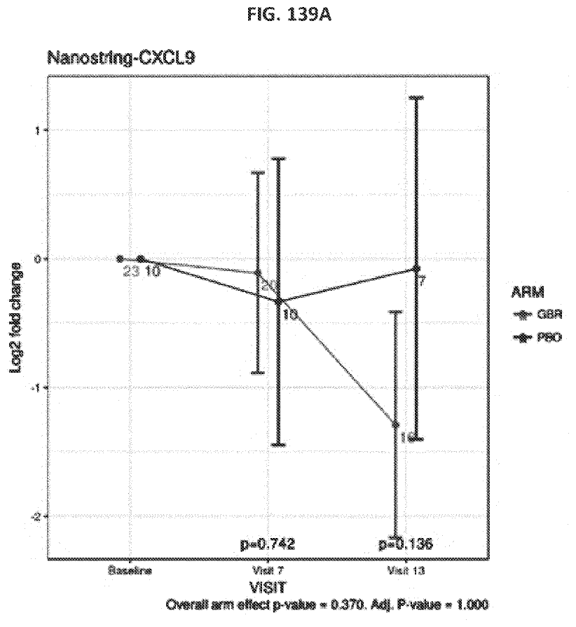

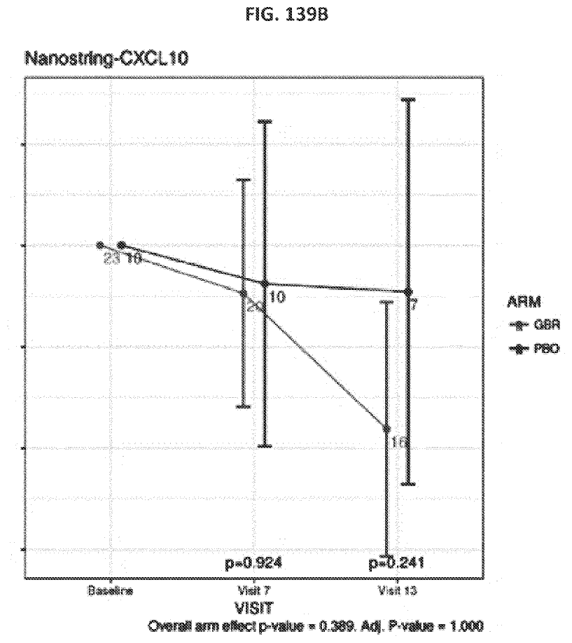

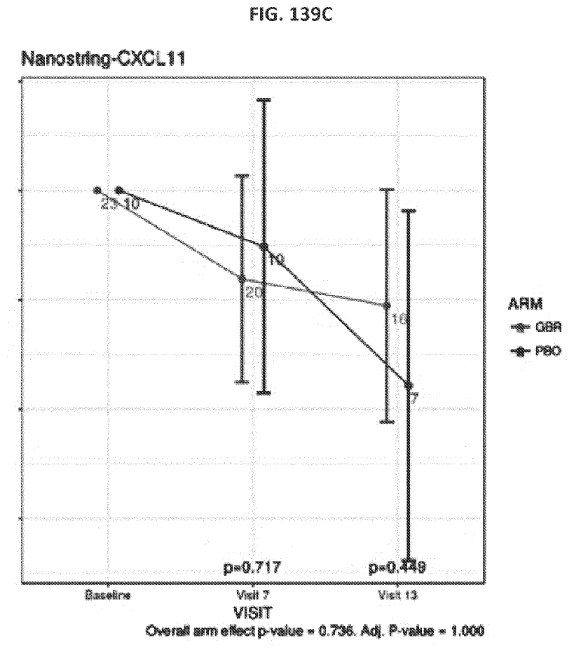

FIG. 139 Measurements of CXCL9, CXCL10, CXCL11 for drug and placebo treated patients at day 29 and Day 71.

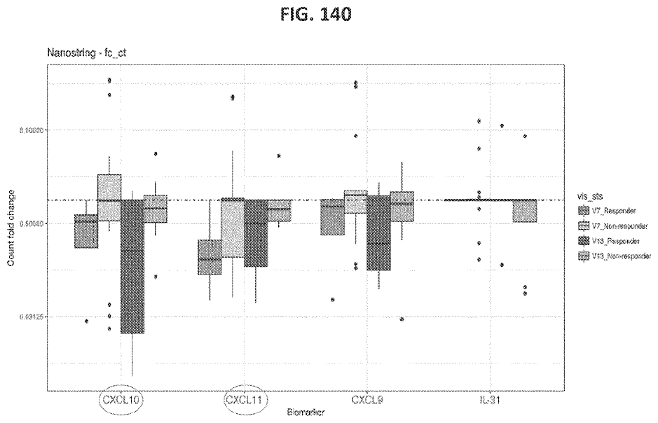

FIG. 140 Boxplot for CXCL10, CLCL11, CXCL9 and IL31

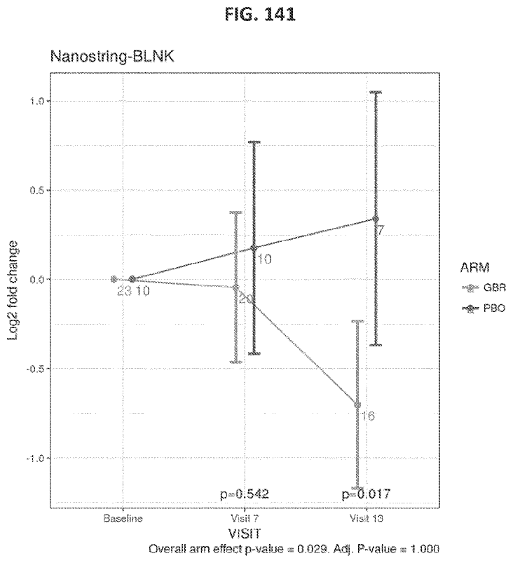

FIG. 141 Measurement of BLNK, for drug and placebo treated patients at day 29 and Day 71.

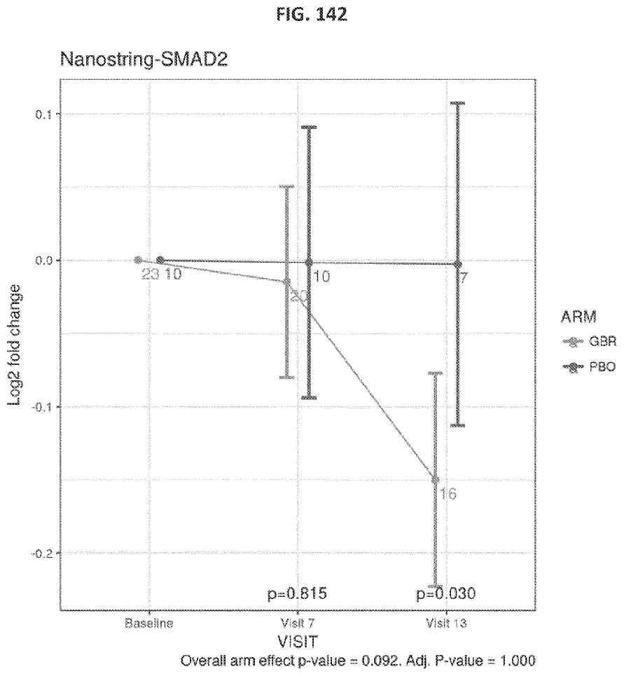

FIG. 142 Measurement of SMAD2, for drug and placebo treated patients at day 29 and Day 71.

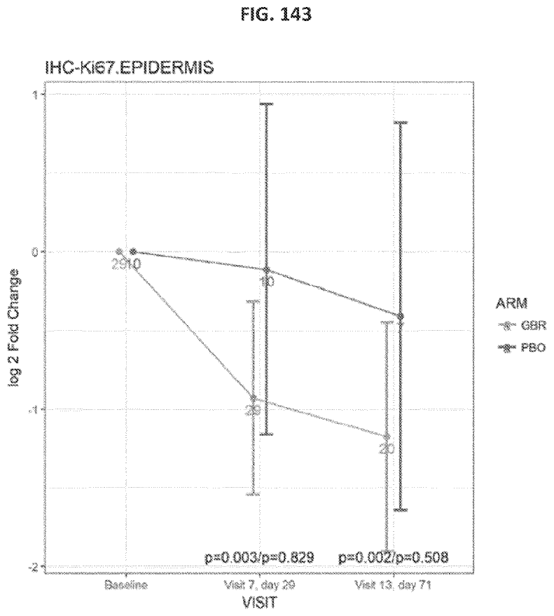

FIG. 143 IHC-Ki67 epidermis measurement for drug and placebo treated patients at day 29 and Day 71.

### Example 1: Clinical Protocol GBR830-201

### Study Objective(s)

### Primary Objective(s)

Safety and tolerability of repeated doses of GBR 830 in adult patients with AD
Effect of repeated doses of GBR 830 on biomarkers of disease activity in adult patients with AD.

### Secondary Objective(s)

Effect of GBR 830 on additional efficacy parameters in adult patients with AD.
PK of repeated doses of GBR 830 in adult patients with AD.
Immunogenicity of GBR 830 in adult patients with AD.

### Exploratory Objective(s)

Additional exploratory objectives to understand the mechanism of GBR 830 including effect on cellular infiltrates and immune pathways

### Study Design

### Study Type/Design

The study is a phase IIa, double-blind, randomized, placebo-controlled, repeated dose study to evaluate safety, biological activity and PK of GBR 830 in adult patients with AD. The study will be conducted in approximately 10 centers in US/Canada. The study will be conducted in three phases: screening phase, treatment phase and follow-up phase.

During the screening phase, after providing informed consent, all patients will be screened for eligibility prior to inclusion in the study and sufficient number of patients will be screened to ensure at least 40 patients meeting the eligibility criteria will be enrolled. At screening, patients will be assessed on EASI, IGA, SCORAD and BSA rating scales for AD. Patients will be withdrawn from use of other medication being used to control their AD as mentioned in prior and concomitant medication section. On Day 1, prior to dosing, patients will be reassessed on EASI, IGA, SCORAD and BSA rating scales for AD to ensure that they qualify for the study.

Approximately 40 patients will be randomized in a ratio of 3:1 to receive GBR 830 (10 mg/kg) or placebo, in a two-arm, parallel design study. Patients who meet eligibility criteria will undergo Day 1/baseline assessments, randomization, and then receive the first IV infusion of GBR 830 or placebo. Each patient will receive two doses of GBR 830 or placebo administered 4 weeks apart on Day 1 and Day 29. Patients will be closely monitored at the study site for 6 hours after the first infusion (Day 1/baseline) and for 3 hours after the next dose (Day 29). The study site will contact patients by telephone approximately 24 hours after each infusion (Days 2 and 30) for concomitant medications and procedures, and a general AE query. Skin punch biopsy samples for biomarker analysis will be collected at Day 1/baseline, Day 29 and Day 71. A gene/mRNA expression profiling will be performed to evaluate the effects of OX40 blockade on both lesional and non-lesional skin from patients with AD. Changes in gene expression in the AD transcriptome of lesional skin in comparison to a non-lesional molecular phenotype will be used to evaluate treatment-associated effects. In addition any correlation with improvements in disease activity and clinical outcomes will also be evaluated.

The end of the study will be the date of the last study visit for the last patient in the study. An overview of the study design is shown in FIG. 1.

### Screening Phase

Screening will occur between Day-30 and Day-1. The purpose of the Screening Visit is to obtain informed consent and to establish protocol eligibility. Informed consent will be obtained after the study has been fully explained to each patient and before the conduct of any screening procedures or assessments. The study participants must be adult male and female patients with AD. The Screening Disposition eCRF page must be completed to indicate whether the patient is eligible to participate in the study and to provide reasons for screen failure, if applicable. At screening, patients will be assessed on EASI, IGA, SCORAD, and BSA for AD. Patients will be withdrawn from use of other medication being used to control their AD as mentioned in prior and concomitant medication section. On Day 1, prior to dosing, patients will be reassessed on EASI, IGA, SCORAD and BSA for AD to ensure that they qualify for the study;

### Treatment Phase

The treatment phase consists of the 2 visits (Days 1 and Day 29) which correspond to the study drug dosing days. Study drug IV infusions will be given on these days. Patients will undergo baseline biopsies on Day 1 (pre-dose).

### Follow-Up

Apart from the dosing visits, patients will be seen in the clinic on Day 4, 8, 15, 22, 32, 36, 43, 50, 57, 71 and the end of study visit occurs on Day 85 (week 12) for study assessments and PK sample collection. Patients will undergo repeat biopsies on Day 29 (pre-dose) and Day 71.

### Discussion of Study Design, Including Choice of Control Groups

The main objective of this phase IIa signal search study is to evaluate the effect of repeated doses of GBR 830 on biomarkers of disease activity in adult patients with moderate to severe AD. The objectives are exploratory in nature to further understand the mechanism of GBR 830 with the help of biomarker data. Recently, improvements of the AD molecular signature were observed in patients after treatment with 4 weeks with Cyclosporine and Dupilumab (Guttman-Yassky E et al; 2014, Hamilton et al 2014), a targeted Th2 antagonist, and these changes occurred earlier and were larger than clinical endpoints, suggesting that these are valid endpoints for an exploratory study. Placebo control will provide internal validity for the clinical trial and will improve the sensitivity of the clinical trial for drug related changes and hence suited for an exploratory study.

### Study Endpoint(s)

### Primary Endpoint(s)

All TEAEs occurring in the study, in terms of nature, onset, duration, severity, relationship and outcome of AEs and SAEs, in adult patients with moderate-to-severe AD.
Effect of GBR 830 in adult patients with AD in terms of change from baseline in the active AD mRNA expression signature and the pathologic epidermal phenotype measures obtained from skin biopsies

### Secondary Endpoint(s)

Proportion of patients who achieve an IGA score of 0 or 1 at each study visit
Proportion of patients who achieve an EASI 50 and 75 response at each studyvisit
Percent improvement in clinical scores EASI, SCORAD, IGA, BSA, Pruritus NRS and DLQI from baseline to each visit
Changes from baseline AD activity as determined by changes in TEWL
PK of GBR 830 in adult patients with moderate to severe AD in terms of: Cₘₐₓ, AUC₀₋ₜₐᵤ, AUC_{0-∞}, and AUC₀₋ₜ, t_{1/2}, volume of distribution, clearance and other parameters assessed as relevant after the first and last doses
Anti-drug antibodies to GBR 830 to evaluate immunogenicity.

### Exploratory Endpoint(s)

Cytokines in serum: IL-13 and IL-22, CCL2, CCL3, CCL4, CCL5, CCL18, CCL20, CCL22, CCL13, CXCL9, CXCL10,CXCL11
Leukocyte sub-population cell counts (Total T, T helper, Cytotoxic T, T_{regs}, Memory T cells, OX40 T cells, GBR 830 T cells).
Cellular infiltrates (T-cells, Dendritic cells) as assessed by CD3, FcEpsilon RI, and OX40L.

Serum TARC, eotaxin-3, total IgE, and circulating eosinophil counts
Percentage OX40 receptor occupancy (RO)

### Appropriateness of Measurements

All clinical assessments are standard measurements commonly used in studies of AD. The safety assessments in this study are standard evaluations to ensure patient safety. The immunogenicity assessment is standard for a monoclonal antibody therapy.

### Patient Selection and Withdrawal Criteria

Approximately 40 patients will be randomized in approximately 10 sites in regions that include US/Canada. Patients who do not meet all of the inclusion criteria or who meet any of the exclusion criteria will not be eligible to receive investigational products.

Patient eligibility should be reviewed and documented by an appropriately qualified member of the Investigator's study team before patients are included in the study.

Patients, who fail screening on any single criterion, where there is the prospect of their subsequently becoming eligible, may be re-screened on 1 occasion only.

The eligibility of a patient with respect to laboratory criteria will be assessed according to the central laboratory result for the screening sample(s).

### Inclusion Criteria

Patients eligible for enrolment in the study must meet all of the following criteria:
1. Male or female patients, age >18 years at the time of informed consent with physician diagnosis of AD for >1 year; diagnosis of AD as defined by the Hanifin and Rajka (Hanifin et al, 1980) criteria for AD.
2. AD involvement of ≥10% body surface area prior to randomization
3. EASI score of ≥12 prior to randomization; SCORAD of ≥20 prior to randomization; baseline IGA score of ≥3 prior to randomization; and history of inadequate response to a stable (>1 month) regimen of class 3 or higher strength topical corticosteroids (TCS), or calcineurin inhibitors or for whom topical treatments are otherwise inadvisable (e.g., because of important side effects or safety risks)*
   *NOTE: For the purpose of this protocol, inadequate response represents failure to achieve and/or maintain remission or a low disease activity state (e.g., IGA 0=clear to 2=mild) despite treatment with topical corticosteroids of medium to high potency (±topical calcineurin inhibitors as appropriate). Inadequacy of response will be determined based on failure to maintain a low disease activity state despite applications of topical medications on a less intensive maintenance schedule (i.e., 2 days per week). Important side effects or safety risks are those that outweigh the potential treatment benefits (e.g., hypersensitivity reactions, significant skin atrophy, systemic effects, etc., or imminence thereof), as assessed by the investigator or by patient's treating physician. Medical history provided by physician and/or patient will be used.
4. Patient's body mass index (BMI) should be within the range 18.5-35 kg/m² (inclusive); weight must be ≥50 kg.
5. Patients deemed fit to receive the study medication, as determined by medical history, vital signs, physical examinations, ECG, laboratory studies, and other tests performed within 30 days prior to drug administration, as judged by the Investigator.
6. Patients must agree to the following requirements during the study:
   a. Women of child-bearing potential and men with partners of child-bearing potential must ensure that two effective means of contraception are used, by them and/or their partners, for the period between signing of informed consent and a minimum of 180 days after the last dose of study drug. Acceptable forms of effective contraception include:
      i. Established use of oral, injected, or implanted hormonal methods of contraception.
      ii. Tubal ligation
      iii. Placement of an IUD or IUS.
      iv. Barrier methods only when used consistently with spermicidal foam/gel/film/cream or suppository. Acceptable barrier methods include the following:
         (1) Male or female condom.
         (2) Occlusive cap (diaphragm or cervical/vault caps).
      v. Male sterilization (with post-vasectomy documentation of the absence of sperm in the ejaculate) (For female patients on the study, the vasectomized male partner should be the sole partner for that patient)
      vi. Maintenance of abstinence when this is in line with the preferred and usual lifestyle of the patient (i.e., not periodic abstinence, such as during ovulation) judged reliable by the Investigator.
      Patients in relation with a same-sex partner do not need to use contraception, if he/she is the sole partner for that patient). Of the acceptable forms of effective contraception, at least one method needs to be a barrier method.
   b. Male patients should agree not to donate sperm until 180 days after administration of the last dose of the study medication. Female patients should not donate eggs for 180 days following investigational product administration.
   c. All female patients must be non-pregnant and non-lactating and test negative for pregnancy at the time of screening and prior to randomization.
7. Female patients of non-child-bearing potential (i.e., are postmenopausal or permanently sterilized [bilateral oophorectomy, hysterectomy, bilateral salpingectomy]). Such patients will not be required to use contraception. Postmenopausal is defined as at least 1 year post cessation of menses (without an alternative medical cause) with follicle stimulating hormone (FSH) ≥40.0 mlU/mL.
8. Provide written informed consent
9. Willing and able to comply with all aspects of the protocol including willingness to undergo 3×2-on study skin biopsies

### Exclusion Criteria

Patients meeting any of the following criteria must not be enrolled in the study:
1. Patients with a history of drug or other allergy considered clinically significant in the opinion of the Investigator, which contraindicates participation or a previous history of hypersensitivity to murine proteins.
2. Patients who have had a live vaccination within 12 weeks before randomization, or intend to have a live vaccination during the course of the study, or have participated in a vaccine clinical trial within 12 weeks prior to randomization
3. History of a serious local infection, systemic infection, or gastrointestinal infection within 12 weeks of baseline; infections requiring systemic antibiotic/anti-viral/anti-parasitic/anti-fungals within 4 weeks of baseline; evidence of clinically significant active infection, or fever ≥38.0° C. (100.4° F.) within one week prior to randomization.
4. Patients who have evidence of active or latent tuberculosis as documented medical history, or test positive for QuantiFERON Gold Blood TB Test
5. Patients with evidence of skin conditions at screening that would interfere with evaluations of the study drug
6. Treatment with systemic corticosteroids within 4 weeks before randomization, and topical steroids/tacrolimus and or/pimecrolimus within 1 week before the randomization (except emollients, and mild steroids (class 6 or 7)
7. Treatment with systemic therapy for AD (such as psoralen and ultraviolet A light therapy, cyclosporine, methotrexate, mycophenolate mofetil, thioguanine, hydroxyurea, sirolimus, azathioprine), or phototherapy (including ultraviolet B or self-treatment with tanning beds or therapeutic sunbathing) within the 4 weeks before randomization or other drugs with potential for immunosuppression such as cytotoxic agents or cyclophosphamide taken within 4 weeks prior to randomization.
8. Previous use of biological mAb therapies within 3 months or five half-lives (whichever is longer) of the drug prior to randomization or have previously used biological therapies or allergen immunotherapy for the treatment of AD.
9. Patients who are immunocompromised, have had a recent (within 3 months before randomization) or current serious systemic or local infection (including infectious mononucleosis-like illness or herpes zoster) suggestive of immunocompromise
10. Patients who have current or a history of lymphoproliferative disease or history of malignant disease; or signs or symptoms suggestive of possible lymphoproliferative disease, including lymphadenopathy or splenomegaly; or active primary or recurrent malignant disease.
11. Patients with history or presence of other inflammatory or auto-immune disease or rheumatological or joint diseases other than AD.
12. History of parasitic infections within 1 year before randomization
13. Patients with a history or current evidence of alcohol abuse including a drinking habit of more than 28 units (males) or more than 21 units (females) of alcohol per week or who have a significant history of alcoholism (one unit of alcohol equals half pint [285 mL] of beer or lager, one glass [125 ml] of wine, or 1/6 gill [25 mL] of spirits).
14. Patients with a history of drug abuse or who test positive for drugs of abuse at screening or prior to randomization considered clinically significant in the opinion of the Investigator or which meets DSM V criteria for addiction or abuse.
15. Patients who test positive for disease markers of HIV, Hepatitis B or Hepatitis C.
16. Patients with an abnormal ECG (including a QTc>450 msec for men, >460 msec for women) considered clinically significant in the opinion of the Investigator at screening and/or prior to randomization. (QTc calculated based on Fridericia's formula).
17. Patients with lab values, which are significantly different from normal reference ranges and/or judged clinically significant by the Investigator, including but not limited to:
   Patients with eGFR <60 mL/min/1.73m2 as determined by the MORD method.
   ALT or AST ≥2.5 times ULN, and/or serum total bilirubin ≥1.5 times ULN, at screening.
   Hb value less than 9 g/dl at screening.
   Absolute neutrophil count ≤1,500/μl or absolute lymphocyte count ≤800/μl or platelet count ≤150,000/μL or any abnormal evaluations judged clinically significant by the Investigator at screening and prior to randomization.
18. Patients with any evidence of organ dysfunction or any clinically significant medical history, or findings in physical examinations or investigations or have a clinical condition or receiving therapy that, in the opinion of the Investigator, would make the patients unsuitable for study.
19. Patients with a history of current or previous psychiatric illnesses or previous psychiatric events that would either put the patient at undue risk or interfere with study procedures according to the investigator.
20. Receipt of an experimental treatment for any disease within 12 weeks prior to randomization

### Study Termination, Patient Discontinuation/Withdrawal Criteria

### Study Termination Criteria

If, in the opinion of the Investigator, the clinical observations in the study suggest that it may be unwise to continue, the Investigator may terminate his participation in the study, after consultation with the Sponsor.

In addition, the Sponsor may terminate part of, or the entire study, for safety or administrative reasons. A written statement fully documenting the reasons for study termination will be provided to the Institutional Review Board (IRB)/Independent Ethics Committee (IEC) and the Regulatory authorities.

### Patient Discontinuation/Withdrawal Criteria

A patient may voluntarily discontinue study participation at any time after giving informed consent and before the completion of the follow-up visit (Visit 14-Day 85). The Investigator may also discontinue the patient's study participation at any time at his/her discretion and for any reason.

The reasons for patient withdrawal will be recorded and may include, but are not limited to:
1. Withdrawal of consent by the patient to continue in the study
2. Development of a serious or intolerable AE that necessitates discontinuation at the discretion of the Investigator (AE section of eCRF must be completed; includes SAE, death)
3. At the discretion of the Investigator, when he/she believes continued participation is not in the best interest of the patient
4. At the discretion of the Investigator, when the patient does not adhere to the study procedures or restrictions
5. Protocol deviation that, in the opinion of the Sponsor and Investigator, warrants discontinuation from the study
6. A positive pregnancy test.
7. A patient requires concomitant medications, which may interfere with the PK of the study drug. Note: withdrawal in such cases will be discussed and mutually agreed by the Investigator and the Sponsor.
8. Patients requiring rescue medications or interventions
9. The patient for any reason requires treatment with another therapeutic agent that has been demonstrated to be effective for the treatment of AD. In this case, discontinuation from the study should occur prior to introduction of the new agent.
10. Disease progression/exacerbation, which in the opinion of the Investigator would require interruption of treatment or premature termination of follow up.

11. Patients randomized in the study who withdraw their consent for the first post-baseline skin biopsies.

The decision to discontinue dosing in a patient due to adverse drug effects will be made on the basis of clinical severity and relatedness to study drug. Except in cases of emergency, it is recommended that the Investigator consult with the Sponsor's medical monitor (MM) before removing the patient from the study. In case of premature discontinuation, the reason and their cause must be documented. The Investigator (or designee) must document the reason for withdrawal in the End of Study section of the eCRF. All Follow-up assessments of Visit 14 (Day 85) should be conducted at the Early Withdrawal Visit. Patients discontinued from the study at any stage will be considered for safety and PK analysis. Patients, who are permanently discontinued from study drug due to reasons other than an AE and before the first post baseline skin biopsies or before receiving two doses of study drug, will be replaced.

### Prior and Concomitant Medication(s)

### Prohibited Prior Medication:

Treatment with systemic corticosteroids within 4 weeks before randomization, and topical steroids/tacrolimus and/or pimecrolimus within 1 week prior to randomization except emollients, and mild steroids (class 6 or 7), applied other than on target area that will be the site for the skin biopsies. Nasal and inhaled corticosteroids use is allowed during study.
Treatment with systemic therapy for AD (such as psoralen and ultraviolet A light therapy, cyclosporine, methotrexate, mycophenolate mofetil, thioguanine, hydroxyurea, sirolimus, azathioprine), or phototherapy (including ultraviolet B or self-treatment with tanning beds or therapeutic sunbathing) within 4 weeks before randomization
Other drugs with potential for immunosuppression such as cytotoxic agents or cyclophosphamide taken within 4 weeks prior to randomization.
Previous use of biological mAb therapies within 3 months or five half-lives (whichever is longer) of the drug prior to randomization or have previously used biological therapies or allergen immunotherapy for the treatment of AD.
Complementary or alternate therapies for the treatment of AD/inflammatory conditions. However patients can be included subsequent to an adequate wash-out period (14 days or five half-lives of the complementary or alternate therapy prior to randomization, whichever is longer).

### Prohibited Concomitant Medication:

All restrictions on the medications listed above in the “prior medication” are applicable for the entire duration of the study

Other concomitant medications that the patient receives on a regular basis may continue if in the opinion of the investigator it does not put the patient at undue risk or nor interfere with the study evaluations. Patients should be stable on allowed concomitant medication for at least 3 months prior to study. All concomitant medications taken by the patient shall be recorded in the patient diary and Prior & Concomitant Medication Forms of eCRF.

### Rescue Medication(s)

In case a patient has a severe flare of disease or severe infection that are deemed by the investigator as necessitating withdrawal from the study and instituting rescue medications, the study medication will be permanently discontinued and the subject will be placed on alternative treatment as soon as possible according to the medical need. These patients will be followed for the whole period of study follow-up (week 12) in order to obtain protocol-specified safety information. Efficacy evaluations will not be performed during this safety follow-up period. If the patient dropped out after Day 29 treatment and biopsies, he will be considered as an evaluable patient for the study.

Lifestyle and/or Dietary Restrictions

Women of child-bearing potential and men with partners of child-bearing capacity must ensure that two highly effective means of contraception are used, by them and their partners, for the period between signing of informed consent and a minimum of 180 days after dosing.

### Contraception

Women of child-bearing potential and men with partners of child-bearing potential must ensure that two highly effective means of contraception are used, by them and/or their partners, for the period between signing of informed consent and a minimum of 180 days after dosing.

Acceptable forms of effective contraception include:
Established use of oral, injected, or implanted hormonal methods of contraception.
Tubal ligation.
Placement of an IUD or IUS.
Barrier methods only when used consistently with spermicidal foam/gel/film/cream or suppository. Acceptable barrier methods include the following:
   male or female condom
   occlusive cap (diaphragm or cervical/vault caps)
Male sterilization (with post-vasectomy documentation of the absence of sperm in the ejaculate) (For female patients on the study, the vasectorized male partner should be the sole partner for that patient).
Maintenance of abstinence when this is in line with the preferred and usual lifestyle of the patient (i.e., not periodic abstinence, such as during ovulation) in a patient judged reliable by the Investigator.

Of the acceptable forms of effective contraception, at least one method needs to be a barrier method. Notes:
Female patients not of childbearing potential (i.e. are postmenopausal or permanently sterilized [bilateral oophorectomy, hysterectomy, bilateral salpingectomy]). Such patients will not be required to use contraception.
Postmenopausal is defined as at least one year post cessation of menses (without an alternative medical cause) with concentrations of FSH ≥40 mlU/mL.
Male patients should not donate sperm for 180 days following investigational product administration. Female patients should not donate eggs for 180 days following investigational product administration.

### TREATMENT OF PATIENTS

### Treatments Administered

GBR 830 is provided as liquid filled vial formulation available in 10 mL volumes containing GBR 830 at concentrations of 10 mg/mL. GBR 830 solution for infusion will be prepared in normal saline.
   Placebo is provided as liquid filled vial formulation available in 10 mL volumes containing the formulation buffer. Placebo solution for infusion will be prepared in normal saline.
The GBR 830 dose will be 10 mg/kg.
   Administration: investigational product will be administered by continuous slow IV infusion over 60 mins using a commercially available, validated, infusion pump.

### Administration

Appropriate aseptic technique should be used while preparing and administering infusions. GBR 830 is provided as liquid filled vial formulation available in 10 mL volumes containing GBR 830 at concentrations of 10 mg/mL.

The investigational product will be diluted with normal saline and administered after normalizing for body weight by continuous slow IV infusion over 60 minutes (+/−5 mins) using commercially available volumetric or syringe infusion pumps. In the event of an infusion reaction, for the purposes of patient safety, the rate of infusion may be decreased and the duration extended at the Investigator's discretion. The pharmacist or designee under the direction of the investigator will dispense study drug for each patient according to the protocol and the randomization number assigned through Interactive voice response system/Interactive web response system (IVRS/IWRS). The diluted investigational product should be used within 24 hours and must be stored at 2 to 8° C. prior to use. Details of the volume of investigational product required, the concentration to be made, the volume of final infusion to be administered, the infusion sets, and material to be used will be described in a pharmacy manual.

### Identity of Investigational Product(s)

### Placebo/Control/Comparator

Placebo will be formulation buffer, diluted in normal saline and administered as IV infusion over 60 mins.

### Packaging and Labelling of Investigational Product(s)

GBR 830 drug product (DP) is formulated as a sterile, clear to slightly opalescent, isotonic, colorless to slightly yellowish, aqueous solution containing no preservatives and buffered to a pH of 6.25 for IV administration after dilution in saline, GBR 830 will be supplied in 10-mL single use vials containing 100.0 mg of GBR 830 (nominal 10 mg/mL). In addition, each unit dose vial contains 15 mM Histidine, 150 mM NaCl, pH 6.25, and 0.01% Tween 80. The GBR 830 solution for IV infusion will be prepared in normal saline (commercially available normal saline [0.9% sodium chloride]. The placebo for infusion is formulation buffer for IV administration after dilution in saline and will be supplied in 10-mL single use vials. Each unit dose vial contains 15 mM Histidine, 150 mM NaCl, pH 6.25, and 0.01% Tween 80. The placebo solution for IV infusion will be prepared in normal saline (commercially available normal saline [0.9% sodium chloride]).

The investigational product vials must be stored refrigerated (2 to 8° C.) and protected from light and moisture in a restricted access room at the clinical site. The vials must be allowed to warm to room temperature prior to dispensing. The DPs will be labeled in accordance with text that is in full regulatory compliance with each participating country and as necessary translated into the required language(s) for each of those countries.

### Allocation to Treatment Groups

At either a separate consent visit or screening visit, potential study patients will be assigned a screening number. Following confirmation of eligibility, patients will be assigned a randomization number through IVRS/IWRS. The randomization scheme and identification for each patient will be included in the final clinical study report (CSR) for this study. The randomization list will be generated using SAS Version 9.1.3 or higher. All eligible patients entering the study will be randomized to the two treatment arms. If a patient discontinues from the study, the patient number will not be re-used and the patient will not be allowed to re-enter the study. Patients will be randomly assigned to receive either GBR 830 or placebo in a 3:1 ratio. A randomization number that uniquely identifies each patient and the patient's treatment will be assigned on Day 1. Randomization numbers will be allocated from the schedule in strict chronological order. A replacement patient will be given the patient number corresponding to the person he/she is replacing plus 100 (e.g. Patient 1101 replaces Patient 1001 etc.) and will receive the same treatment. Randomization will be done using IVRS/IWRS software.

### Blinding and Unblinding Procedures

The study will be conducted in a double-blind manner. The sponsor will be blinded to the identity of the investigational product and all study data. In the event of a medical emergency when management of a patient's condition requires knowledge of the trial medication, IVRS/IWRS will be used to determine the nature of the trial medication dispensed. If possible, such emergencies should be discussed with the study monitor and the Sponsor prior to disclosure of the treatment allocation. Reasons for breaking a code must be clearly explained and justified in the eCRF. The date on which the code was broken together with the identity of the person responsible must also be documented.

The following controls will be employed to maintain the double-blind status of the study:
The placebo will be identical in appearance to the GBR 830 infusion
The Investigator and other members of staff involved with the study will remain blinded to the treatment randomization code during the assembly procedure
Any interim data will be provided in a blinded manner

With the exception of the statistician (who is not a study team member) preparing the randomization and personnel involved in packaging, all clinical and non-clinical staff will remain blinded to the treatment allocation until after the database is locked unless there is a medical event that requires a code break.

### Timing of Study Procedures and Assessments

The visit windows are mentioned below.

Window for collection of samples for GBR-830 PK analysis:
V2 and V7:
   Pre-dose: 15 minutes before administration of each dose
   Post-dose:
      immediately at the end of each infusiont ±10 mins;
      1.5 hrs±10 mins estimated from the start time of infusion
      2 hrs±10 mins estimated from the start time of infusion
      4 hrs±10 mins estimated from the start time of infusion
V3 and V8: 72 hrs±24 hrs estimated from the start time of infusion
V4 and V9: 168 hrs±24 hrs estimated from the start time of infusion
V5 and V10: 336 hrs±24 hrs estimated from the start time of infusion
V6 and V11: 504 hrs±48 hrs estimated from the start time of infusion
V12, V13, V14: 1344 hrs (Day 57)±48 hrs, 1680 hours (Day 71)±48 hrs, and 2016 hours (Day 85)±48 hrs estimated from the start time of first infusion

### Window for Vital Signs:

V2 and V7:
   Pre-dose: 30 minutes before administration of each dose
   Post-dose:
      Every half hour during infusion ±10 mins;
      0.5 hrs±10 mins estimated from the end time of infusion
      1 hr±10 mins estimated from the end time of infusion
      2 hrs±10 mins estimated from the end time of infusion
      3 hrs±10 mins (In V2 only) estimated from the end time of infusion
      6 hrs±10 mins (In V7 only) estimated from the end time of infusion
V3 and V8: 72 hrs±24 hrs estimated from the start time of infusion
V4 and V9: 168 hrs±24 hrs estimated from the start time of infusion
V5 and V10: 336 hrs±24 hrs estimated from the start time of infusion
V6 and V11: 504 hrs±48 hrs estimated from the start time of infusion
V12, V13. V14: 1344 hours (Day 57)±48 hrs, 1680 hours (Day 71)±48 hrs, and 2016 hours (Day 85)±48 hrs estimated from the start time of infusion

### For All Other Safety and Pharmacodynamics/Biomarker/Immunogenicity Assessments:

V2 and V7:
   Pre-dose: 60 minutes before administration of each dose
   Post-dose: =30 mins estimated from the end time of infusion
V3 and V8: 72 hrs±24 hrs estimated from the start time of infusion
V4 and V9: 168 hrs±24 hrs estimated from the start time of infusion
V5 and V10: 336 hrs±24 hrs estimated from the start time of infusion
V6 and V11: 504 hrs±48 hrs estimated from the start time of infusion
V12, V13. V14: 1344 hours (Day 57)±48 hrs, 1680 hours (Day 71)±48 hrs, and 2016 hours (Day 85)±48 hrs estimated from the start time of first infusion
   in the event that assessments are planned for the same scheme time, the order of the assessments should be arranged in such a way that PK blood sampling will be performed first, followed by ECG and vital signs, with blood sampling exactly on time. Samples collected outside the window period will be reported as protocol deviations and the actual time point of sampling will be recorded

### Screening: Visit 1

Patients will go to the site for a screening visit up to 30 days prior to study drug administration Day 1. Informed consent must be obtained at this visit prior to any study procedures are performed. A screening log will be kept to record patients who sign the informed consent form (ICF) and who are screened. For those patients who are screen failures, a reason for the failure will be documented. Prior to performing any procedures or assessments, the nature of the study and the potential risks associated with the study must be explained to the patient and written informed consent must be obtained. Once informed consent has been obtained, the following procedures and evaluations will be performed and recorded. Patient will be trained on the use of diary.

Patient will be instructed to enter the data every morning at a designated time and how to record them. Patient must enter data into the diary every day from start of screening period to end of study visit.
Demographic information (Date of birth and/or age, race and ethnicity, gender)
Height and body weight (measured while wearing indoor clothing and no shoes)
BMI, calculated as weight (kg)/height (m)²
Smoking status/intake of tobacco in any other form (including current and historical use of tobacco)
Alcohol and drug abuse status/intake in any form
Medical and surgical history
Prior and concomitant medications, and AEs
Detailed physical examination
Vital signs (pulse, BP-blood pressure, temperature)
Determine inclusion/exclusion criteria
12-lead ECG
Clinical laboratory tests (collected under fasting conditions; blood sample for hematology, biochemistry tests and urine sample for urinalysis).
Viral serology (hepatitis B surface antigen [HBsAg], anti-hepatitis B core antigen [Anti-HBc], hepatitis C antibody, human immunodeficiency virus [HIV-1and HIV-2])
Evidence of active or latent tuberculosis to be documented by medical history or QuantiFERON Gold Blood TB Test. A chest X-ray is not mandatory. QuantiFERON Gold Blood TB Test will be performed in all patients
Serum Pregnancy test for females (Beta-HCG), serum FSH test for post-menopausal women
EASI, SCORAD, IGA, BSA measurements

One re-test will be allowed at screening for investigations other than viral serology at the discretion of the investigator in order to confirm findings for clinical conditions that are considered to be acute, reversible, and non-serious.

### Dosing Visits (Visits 2 and 7)

The visit 2 will be the baseline visit. Patients will arrive at the study site on the day of dosing (Day 1, Visit 2 and Day 29+1, Visit 7) and will be closely monitored at the study site for 6 hours after the first injection (Day 1/baseline, Visit 2) and for 3 hours after the next dose (Day 29, Visit 7). The following procedures will be conducted and recorded:
Eligibility criteria and randomization procedures including pre-study restrictions (Visit 2 only)
Pre-dose drug and alcohol screen
Body weight and BMI
Pre-dose brief physical examination of major body systems
Pre- and post-dose vital signs: includes supine measurements BP and pulse; and body temperature. Vitals have to be monitored every half hour during infusion
Pre- and post-dose 12-lead ECG
Pre- dose blood samples for routine hematology, biochemistry and urine sample taken for routine urine analysis
Pre-dose blood samples for serum pregnancy test for females (Beta-HCG)-(Visit 2 only)
Pre-dose urine pregnancy for females only
Administration of a single 1-hour IV infusion of IP administered under the supervision of trained study personnel
Pre-dose EASI, SCORAD, DLQI, IGA, NRS, BSA measurements
Blood sample for pre-and post-dose GBR-830 PK analysis
Pre-dose skin biopsies
RO assay, TEWL, total IgE, eosinophil count
Pre-dose blood samples for immunogenicity, cytokine and exploratory analysis
Concomitant medications and AEs

### Follow-Up Visits 3 and 8 (Days 4±1 and 32±1)

Vital signs (pulse, BP, temperature)
12-lead ECG
Clinical laboratory tests (blood sample for hematology, biochemistry and urine sample for urinalysis)
EASI, SCORAD, DLQI, IGA, NRS, BSA measurements
Concomitant medications and AEs
Blood sample for GBR-830 PK analysis

### Follow-Up Visits 4 and 9 (Days 8±1 and 36±1)

Vital signs (pulse, BP, temperature)
12-lead ECG
Clinical laboratory tests (blood sample for hematology, biochemistry; and urine sample for urinalysis)
EASI, SCORAD, DLQI, IGA, NRS, BSA measurements
Concomitant medications and AEs
Blood sample for GBR-830 PK analysis
RO assay, TEWL, total IgE, eosinophil count

### Follow-Up Visits 5 and 10 (Days 15±1 and 43±1)

Brief physical examination of major body systems
Vital signs (pulse, BP, temperature)
EASI, SCORAD, DIQI, IGA, NRS, BSA measurements
Concomitant medications and AEs
Blood sample for GBR-830 PK analysis
Blood samples for immunogenicity analysis in visit 5 (Day 15)
RO assay, TEWL, total IgE, eosinophil count

### Follow-Up Visits 6 and 11 (Days 22±2 and 50±2)

Vital signs (pulse, BP, temperature)
EASI, SCORAD, DLQI, IGA, NRS, BSA measurements
Concomitant medications and AEs
Blood sample for GBR-830 PK analysis

### Follow-Up Visits 12 and 13 (Days 57±2 and 71±2)

Vital signs (pulse, BP, temperature)
12-lead ECG
Clinical laboratory tests (blood sample for hematology, biochemistry and urine sample for urinalysis)
EASI, SCORAD, DLQI, IGA, NRS, BSA measurements
Concomitant medications and AEs
Blood sample for GBR-830 PK analysis
Blood samples for immunogenicity analysis in visit 12 (Day 57)
Blood samples for cytokine analysis and exploratory analysis on visit 13 (Day 71)
Skin biopsies on visit 13 (Day 71)
RO assay, TEWL, total IgE, eosinophil count
   Follow-Up Visits (end of study) assessments on Day 85 (±2) include the following:
Body weight and BMI
Brief physical examination of major body systems
Vital signs (pulse, 8P, temperature)
12-lead ECG
Clinical laboratory tests (blood sample for hematology, biochemistry and urine sample for urinalysis)
Blood sample for serum pregnancy tests
EASI, SCORAD, DLQI, IGA, NRS, BSA measurements
Concomitant medications and AEs
Blood sample for GBR-830 PK analysis
Blood samples for cytokine and exploratory analysis
Blood samples for immunogenicity analysis
RO assay, TEWL, total IgE, eosinophil count

### Early Withdrawal Visit

The Early Withdrawal Visit will be performed as applicable. The end of study assessments will be performed for all patients receiving study drugs who withdraw prematurely from the study. In addition, for patients who will be monitored for entire duration of study, the following safety and PK parameters will be evaluated.
Brief physical examination of major body systems
Vital signs (pulse, BP, temperature)
12-lead ECG
Clinical laboratory tests (blood sample for hematology, biochemistry and serum pregnancy tests and urine sample for urinalysis)
Concomitant medications and AEs
Blood sample for GBR-830 PK analysis
Blood samples for immunogenicity analysis

### Telephone Monitoring

For patients who do not make scheduled study visits or are lost to follow-up a telephone follow up has to be done to evaluate the reason for non-compliance. The study site will contact patients by telephone approximately 24 hours after each infusion of study drug for concomitant medications and procedures and general AE query. Written documentation must be maintained for all such communications with the patient.

### Study Procedures and Assessments

### Demographic and Other Pretreatment Assessments

### Demography

Patient demography information will be collected at the Screening visit. Demographic information includes date of birth (or age), gender, race/ethnicity, height and weight.

### Medical History and Physical Examinations at Screening

Medical and surgical history and current medical conditions will be recorded at the Screening Visit. Smoking, alcohol and drug abuse history will also be recorded. All relevant medical and surgical history must be noted in the Medical and Surgical History eCRF form.

Screening Physical examinations will be comprehensive and documentation of the physical examination will be included in the source documentation at the site. Significant findings at the Screening Visit will be recorded on the Medical and Surgical History eCRF form.

### QuantiFERON Gold Blood TBTest

A whole blood sample will be collected from each patient at the screening visit for the QuantiFERON Gold Blood TB Test. Detailed instructions for blood sample collection, preparation, and shipping are provided in the central laboratory manual.

In addition, eligibility criteria will be assessed at baseline prior to randomization.

### Efficacy Assessments

### EASI

The EASI is a validated measure used in clinical practice and clinical trials to assess the severity and extent of AD. Four AD disease characteristics will be assessed for severity by the Investigator or designee on a scale of “0” (absent) through “3” (severe). In addition, the area of AD involvement will be assessed as a percentage by body area of head, trunk, arms, and legs and converted to a score of 0 to 6 (Hanifin, 2001).

### SCORAD

The SCORAD is a validated tool used in clinical research and clinical practice that was developed to standardize the evaluation of the extent and severity of AD (Dermatology 1993). The extent of AD is assessed as a percentage of each defined body area and reported as the sum of all areas, with a maximum score of 100% (assigned as “A” in the overall SCORAD calculation). The severity of 6 specific symptoms of AD is assessed using the following scale: none (0), mild (1), moderate (2), or severe (3) (for a maximum of 18 total points, assigned as “B” in the overall SCORAD calculation). Subjective assessment of itch and sleeplessness is recorded for each symptom by the patient or relative on a visual analogue scale (VAS), where 0 is no itch (or sleeplessness) and 10 is the worst imaginable itch (or sleeplessness), with a maximum possible score of 20. This parameter is assigned as “C” in the overall SCORAD calculation. The SCORAD is calculated as: A/5+7B/2+C (Kunz et al, 1997).

### IGA

The IGA is an assessment scale used in clinical studies to determine severity of AD and clinical response to treatment based on a 5-point scale ranging from 0 (clear) to 5 (severe/very severe). The proportion of patients who achieve an IGA 0 or 1 score is another key secondary endpoint, which will be included in the primary analysis.

### NRS

Pruritus Numerical rating scale (NRS): Patients will record once daily and respond to the following question, “On a scale of 0-10, with 0 being no itch and 10 being the worst itch imaginable, how would you rate your worst degree of itch during the previous 24 hours?” Patient compliance on the pruritus NRS will be followed at each clinic visit.

### DLQI

Dermatology Life Quality Index (DLQI): The DLQI is a simple, patient-administered, 10-question, validated, quality-of-life questionnaire that covers 6 domains including symptoms and feelings, daily activities, leisure, work and school, personal relationships, and treatment. Response categories include “not at all,” “a lot,” and “very much” with corresponding scores of 1, 2, and 3 respectively and unanswered (“not relevant”) responses scored as “0.” Totals range from 0 to 30 (less to more Impairment) and a 5-point change from baseline is considered clinically relevant (Basra et al, 2008; Finlay et al, 1994).

### Pharmacokinetic, Pharmacodynamic, Biomarker and Pharmacogenomic Assessments

### PharmacokineticAssessments

Blood samples will be collected as per routine phlebotomy procedures. Briefly, blood samples (1×3.5 ml each) will be collected during the course of the study through indwelling cannula placed in forearm veins or alternatively, by a fresh dean venipuncture using a disposable sterilized syringe and a needle. The cannulae will be maintained patent as per local practice. Do not use heparin. The minute of collection of each blood sample will be recorded. In any case actual time points will be used during PK calculations. The details of sample collection, processing and storage will be outlined in a separate lab manual. The samples will be shipped to the specified bioanalytical lab. Serum concentrations of GBR 830 will be quantified using a validated Enzyme-linked immunosorbent assay (ELISA) method.

### Immunogenicity Assessments

Blood samples will be collected to evaluate anti-drug antibodies to GBR 830, as per procedures similar to collection of PK samples. Antibodies to GBR 830 will be detected and confirmed using a validated ELISA method. The details of sample collection, processing and storage will be outlined in a separate lab manual. The samples will be shipped to the specified bioanalytical lab

### Biomarker Assessments

Flow cytometry/Receptor Occupancy Assay

Blood samples will be collected at appropriate time points.

### Cytokine

Blood samples will be collected at appropriate time points.

### TARC (CCL17)

Thymus and activation-regulated chemokine is a chemokine, shown to be strongly associated with disease severity in AD, and may be involved in pathogenesis of the disease. Baseline TARC levels will be assessed for potential predictive value for treatment response. Post-treatment samples will be evaluated for effects of GBR 830 on TARC.

### Eotaxin-3 (CCL26)

Eotaxin-3 is a chemokine, shown to be associated with disease severity in AD, and may be involved in pathogenesis of the disease. Baseline eotaxin-3 levels will be assessed for potential predictive value for treatment response. Post-treatment samples will be evaluated for effects of GBR 830 on eotaxin-3.

### Total IgE

Patients with AD often have elevated IgE. Total IgE levels have been found to modestly correlate with AD severity and may be involved in the pathogenesis of the disease. Changes in total IgE reflects not only on AD, but atopy in general. Baseline IgE levels will be assessed for potential predictive value for treatment response. Post-treatment samples will be evaluated for effects of GBR 830 on total IgE.

### Transepidermal Water Loss

Transepidermal water loss is a skin barrier function test that measures perspiration or water loss through the skin. This procedure involves the non-invasive application of a probe on the surface of the skin on the arm or leg. Affected and non-affected areas of skin will be tested. This procedure will only be performed at specified study centers. The detailed procedure for TEWL will be provided in the Study Reference Manual.

### Immunohistochemistry (IHC)

Two punch biopsies (1 from LS and 1 from NLS) will be collected. For LS, biopsy should be taken from a target lesion initially and always taken from the same lesion or comparable lesion thereafter. A 4.5 mm punch biopsy should be taken from the most involved chronic active erythematous, scaly lesions. For NLS, a 4.5 mm sample should be collected from the most normal appearing skin in a relative proximity to the LS biopsy site, at least 5 cm away from the lesion (at least 1 cm away, if 5 cm is not possible). Full details of sample collection, processing and storage will be outlined in a separate lab manual.

### RT-PCR and Gene Microarray

Skin biopsy samples, as collected and mentioned previously will also be used for RT-PCR and gene microarray. The detailed methodology will be outlined in the lab manual.

### Safety Assessments

Safety assessments will consist of monitoring and recording all AEs and SAEs; regular monitoring of hematology, blood chemistry, and urinary laboratory values; periodic measurement of vital signs and ECGs; and performance of physical examinations. At the end of the study another clinical assessment consisting of a physical examination and all laboratory tests performed at the time of screening (except viral serology, and FSH) will be performed. Dosing will be based on evaluations performed by physicians/Investigator. Additional assessments can be integrated into the protocol further to investigator judgment.

### Data Analysis and Statistical Methods

A Statistical Analysis Plan (SAP) will be written to provide details of the analysis, along with specifications for tables, listings, and figures to be produced. The SAP will be finalized prior to the database lock at the latest. Any changes from the analyses planned in the SAP will be justified in the CSR. All analyses will be performed using SAS® Version 9.1.3 or above. Prior to database lock, a final blinded data review meeting will be held to allow a review of the clinical study data and to verify the data that will be used for analysis set classification. A meeting to determine analysis set classifications may also be held prior to database lock. In general, all data will be summarized with descriptive statistics (number of patients, mean, standard deviation, minimum, median and maximum) for continuous endpoints, and frequency and percentage for categorical endpoints. The results of the study will be reported in CSR in accordance with the ICH guidance.

### Sample Size

No formal sample size calculation will be performed for this study. The sample size chosen is based on experience from previous studies of similar nature. Patients, who are permanently discontinued from study drug due to reasons other than an AE and before the first post baseline skin biopsies (Visit 7) or before receiving two doses of study drug (Visit 7), will be replaced. The sample size of 40 adult patients with AD randomized in ratio of 3:1 (GBR 830 vs placebo) is considered to be sufficient to provide descriptive information on the PK, safety, tolerability and potential efficacy of GBR 830.

### Analysis Sets

Detailed criteria for analysis sets will he documented in the SAP and the allocation of patients to analysis sets will be determined prior to database hard-lock.

### Full Analysis Set (FAS)

The Full Analysis Set (FAS) will consist of all patients who are randomized and 1 dose of IP and have at least 1 post baseline gene expression assessment. The primary analyses will be based on FAS

### Safety Analysis Set (SAF)

The Safety Analysis Set (SAF) consists of all patients who took at least 1 dose of study medication, and will be used for safety analyses.

### Pharmacokinetic Analysis Set (PKAS)

The Pharmacokinetic Analysis Set (PKAS) consists of the subset of the SAF population for which sufficient serum concentration data is available to facilitate derivation of at least 1 PK parameter and for whom the time of dosing on the day of sampling is known. Additional patients may be excluded from the PKAS at the discretion of the pharmacokineticist. Any formal definitions for exclusion of patients or time-points from the

PKAS will be documented in the SAP.

### Patient Disposition

Data on patient disposition (number of patients enrolled, number of drop-outs, and reasons for drop-out), demographics (gender, age, height, weight, BMI), and other baseline characteristics will be summarized. The safety, tolerability, PK, and other data from each part of the study will be listed and summarized descriptively.

The number (percentage) of patients who were screened for the study (Enrolled Patients, i.e., those who signed informed consent) and reasons for screen failure will be described.

### Demographic and Other Baseline Characteristics

Demographics and other baseline characteristics will be summarized by treatment group. Descriptive statistics will include number of patients, mean, standard deviation, minimum, median and maximum for continuous variables, and frequency and percentage for categorical variables. Continuous demographic and baseline variables include age, height and body weight, and BMI; categorical variables include gender, race, and ethnicity.

### Efficacy Analyses

Analysis will be conducted on the FAS. The interpretation of results from statistical tests will be based on the FAS.

### Analysis of Primary Efficacy Endpoint(s)

### Primary Analysis

All continuous efficacy variables will be analyzed using an analysis of covariance (ANCOVA) model with treatment as the fixed effects, and using the relevant baseline value as a covariate. Differences between treatment groups and confidence intervals will be estimated within the framework of ANCOVA. In the event that the model assumptions are not warranted, the rank-based ANCOVA will be used.

### Analysis of Secondary Efficacy Endpoint(s)

Categorical analyses will be performed on responders (e.g., percentage of patients with responding rates of 50% at the end of week 12). Comparisons between GBR 830 treatment and placebo groups will be done using a Cochran-Mantel-Haenszel test. For a patient, the efficacy data will be set to missing after prohibited medication is used. The last observation carried forward (LOCF) method will be used to impute missing values.

### Analysis of Exploratory Efficacy Endpoint(s)

All exploratory efficacy analyses will be performed on the FAS, and no multiplicity adjustment is planned. Analyses of exploratory endpoints will be provided in the SAP.

### Pharmacokinetic, Pharmacodynamic, Biomarker, and Pharmacogenomic/Pharmacogenetic Analyses

### Pharmacokinetic Analyses

Pharmacokinetic parameters will be summarized in tabular and graphic form. Cmax, Tmax, AUC0-∞, AUC0-tau, and AUC0-t, will be estimated after the first and last dose administrations. Parameters like t1/2, volume of distribution, clearance and other relevant parameters may be assessed after the first and/or last dose administrations, if possible depending on the data. Pharmacokinetic parameters will be calculated using Phoenix™ WinNonlin® Version 6.3 (Pharsight Corporation). Results of exploratory analyses will be summarized. Details will be discussed in the SAP for this study.

### Immunogenicity Analyses

Percentage of patients with positive and negative anti-drug antibody titers will be tabulated by treatment and time point. The neutralizing antibody status would also be reported where applicable.

### Biomarker Analyses

Informal exploratory biomarker analyses may be performed while the study is ongoing. No one involved in the day-to-day conduct of the study will have access to biomarker data before the database is locked for this study. The analysis of biomarker data will not impact any decisions regarding study conduct. All exploratory efficacy analyses will be performed on the FAS, and no multiplicity adjustment is planned. Analyses of exploratory endpoints will be provided in the SAP.

### Safety Analyses

All safety analyses will be performed on the Safety Analysis Set.

### Extent of Exposure

40 adult patients with AD randomized in a ratio of 3 active: 1 placebo

### Adverse Events

Adverse events will be coded using the Medical Dictionary for Regulatory Activities (MedDRA). The number and percentage of AEs, SAEs, AEs leading to discontinuation, and AEs related to investigational product will be summarized by system organ class, preferred term and treatment group. Patients will be counted only once for each preferred term, system organ class, and by the highest severity of an event. The number and percentage of AEs by severity will also be summarized. All AEs will be displayed in listings.

### Laboratory Values

For quantitative laboratory measurements descriptive statistics will be used to summarize results and change from baseline by treatment group and time point. Shifts in laboratory tests relative to normal ranges from baseline to each time point during treatment will also be tabulated. All laboratory data will be displayed in listings.

### Vital Signs

Descriptive statistics will be used to summarize vital sign results and changes from baseline by treatment group and time. Values of potential clinical significance will be tabulated. All vital signs data will be displayed in listings. Shift tables will present changes from baseline (categorized as normal; abnormal, not clinically significant; and abnormal, clinically significant) to end of treatment (or end of phase or by visit).

### Electrocardiograms

All ECG variables will be presented by visit. Descriptive statistics for ECG parameters and changes from baseline will be presented by treatment group.

Shift tables will present changes from baseline in ECG interpretation (categorized as normal; abnormal, not clinically significant; and abnormal, clinically significant) to end of treatment (or end of phase or by visit).

### Physical Examination

Descriptive statistics will be used to summarize findings of potential clinical significance and will be listed. Investigator should inform the Institution where applicable, and the Investigator/Institution should promptly inform the Sponsor and the IRB/IEC and provide the Sponsor and the IRB/IEC with a detailed written explanation of the termination or suspension. Study records must be retained as noted above.

### Example 2: GBR830 in Adult Patients with Moderate-to-Severe AD

### Study Summary

The following study has been carried out according to the protocol described in Example 1. In particular:

### Study Design

This was a phase 2a, randomized, double-blind, placebo-controlled, repeated dose study (NCT02683928), conducted in 17 centers in US/Canada from March 2016 to June 2017 to evaluate safety and biological activity of GBR830 in adult subjects with AD. The study protocol and informed consent were approved by local institutional review boards. The study was conducted in accordance with Good Clinical Practice and the Declaration of Helsinki and all subjects provided written informed consent prior to entering the study. The study consisted of a screening phase (up to 30 days) followed by treatment (Day 1 [baseline] and Day 29) and follow-up (through Day 85).

### Participants

Adult subjects (≥18 years) with moderate-to-severe AD who met the following criteria were included in the study: physician diagnosis of moderate-to-severe atopic dermatitis for >1 year; affected body surface area (BSA) ≥10%; Eczema Area and Severity Index (EASI) score ≥12; Scoring of Atopic Dermatitis (SCORAD) ≥20; investigator's global assessment (IGA) score ≥3 (5-point scale); and history of inadequate response, defined as a failure to achieve and/or maintain remission, or low disease activity to a stable regimen (>1 month) of class ≥3 strength topical corticosteroids or calcineurin inhibitors and inadvisable for topical treatments.

### Randomization, Treatment, and Blinding

Eligible subjects were randomized 3:1 to receive intravenous GBR830 (10 mg/kg) or corresponding placebo using a computer-generated scheme reviewed and approved by an independent statistician. Subjects received two intravenous infusions of GBR830 or placebo on Days 1 and 29, FIG. 1). Subjects were closely monitored at the sites for 6 hours after the first infusion and for 3 hours after the Day 29 dose. Study drugs were identical in appearance.

### Study Endpoints

Primary endpoints included treatment-emergent adverse events (TEAEs) and change from baseline in epidermal hyperplasia and in the active AD mRNA expression signature, measured from lesional skin biopsies. Secondary endpoints included: percent improvement from baseline in SCORAD, IGA, BSA, EASI score; EASI50 and EASI75 responses, defined as ≥50% and ≥75% score improvement from baseline, respectively; and IGA score of 0 or 1. Pruritus Numerical Rating Scale (NRS) and Dermatology Life Quality Index (DLQI) changes from baseline to each visit were also assessed. Safety assessments included vital signs, physical examinations, laboratory evaluations, and electrocardiograms.

### Statistical Analyses

This study was not powered to detect differences between treatment groups. Clinical efficacy was analyzed in the intent-to-treat (ITT) population, defined as all randomized subjects who received ≥1 partial or full study treatment dose. Biomarker analyses of disease activity obtained from skin biopsies were based on the Biological Activity Set (BAS) population, which consisted of ITT subjects who received both doses of study drug and provided one baseline and ≥1 post-baseline skin biopsy. The safety population included all subjects with ≥1 partial or full study treatment dose. The number of samples at different time points for each population is detailed in Table 1. Percent changes in the severity scores (SCORAD) of the ITT subjects were estimated over time using a mixed model repeated measure (MMRM) approach, with Time and Treatment as fixed factors and a random intercept for each subject.

TABLE 1
Number of samples at different time points.

| | Subjects with Biopsies (BAS) | | | | Subjects with Clinical Data (ITT) | | | |
|---|---|---|---|---|---|---|---|---|
| Subjects, n (%) | GBR830 | | Placebo | | GBR830 | | Placebo | |
| Day 1 (baseline) | 29 | (100) | 11 | (100) | 46 | (100) | 16 | (100) |
| Day 29 | 29 | (100) | 11 | (100) | 39 | (84.7) | 15 | (93.7) |
| Day 71 | 21 | (72.4) | 7 | (63.6) | 26 | (56.5) | 8 | (50.0) |

BAS, Biological Activity Set;
ITT, intent-to-treat.

### Biomarker Analysis of Skin Biopsies

Biopsies were obtained from lesional skin on Days 1, 29, and 71 and non-lesional (>10 cm from active lesions) at Day 1. Baseline biopsies (Day 1) were obtained prior to the first dose. Immunohistochemistry staining was performed on frozen sections using purified mouse anti-human monoclonal antibodies (Table 2). Epidermal thickness and cell counts were quantified with Image V1.42 (National Institutes of Health, Bethesda, MD). RNA was extracted and quantitative real-time polymerase chain reaction (RT-PCR) was used to assess mRNA expression (primers are listed in Table 3). Immunohistochemistry and RT-PCR data in the BAS population were log2-transformed prior to statistical analysis. To account for missing data points between baseline and end of treatment, data was analyzed using a linear mixed effect model with Time, Tissue, and Treatment as fixed factors and a random intercept for each subject. Hypothesis testing was conducted with contrasts under the general framework for linear models in the R nime package. Results are presented as fold changes (FCHs) between post-treatment biopsy timepoints (Day 29 and 71) and baseline (Day 1).

TABLE 2
Purified mouse anti-human monoclonal antibodies for immunohistochemistry staining

| Antibody | Vendor | Clone | Isotype | Dilution |
|---|---|---|---|---|
| Ki67 | Santa Cruz Biotechnology | MIB-1 | Mouse IgG_{1K} | 1:25 |
| OX40L | R&D Systems | 159403 | Mouse IgG_{1K} | 1:50 |
| OX40 | BD Biosciences | ACT35 | Mouse IgG_{1K} | 1:50 |

TABLE 3
Primers

| Gene Symbol | Sequence |
|---|---|
| hARP | Forward CGCTGCTGAACATGCTCAA |
| | Reverse TGTCGAACACCTGCTGGATG |
| | Probe 6-FAM-TCCCCCTTCTCCTTTGGGCTGG- |
| | 2TAMRA |
| | Assay ID |
| K16 | Hs00955082_g1 |
| IL-8 | Hs00174103_m1 |
| IL-23p19 | Hs00900828_g1 |
| IL-31 | Hs01098710_m1 |
| IFNγ | Hs00989291_m1 |
| S100A9 | Hs00610058_m1 |
| S100A12 | Hs00942835_g1 |
| CCL17 | Hs00171074_m1 |
| CCL11 | Hs00237013_m1 |
| CXCL10 | Hs01124251_g1 |
| TSLPR | Hs00845692_m1 |

### Exclusion Criteria

Patients were excluded from participation for any of the following reasons: history of drug or other allergy considered clinically significant by the Investigator; live vaccination within 12 weeks before randomization; history of serious infection, including latent or active tuberculosis; skin conditions at screening that would interfere with study drug evaluations; immunocompromised or current serious systemic or local infection suggestive of immunocompromise; history of lymphoproliferative, malignant, inflammatory, auto-immune, or rheumatological disease; and prior treatment with systemic corticosteroids, topical steroids/tacrolimus and/or pimecrolimus, phototherapy, immunosuppressive drugs (i.e., cytotoxic agents, cyclophosphamide), cell-depleting agents (i.e., rituximab), biologics, and/or allergen immunotherapy.

### Sample Size Determination

No formal sample size calculations were performed. The same size was chosen based on experience from previous studies of similar nature. A sample size of 40 with a 3:1 GBR830-to-placebo randomization ratio was considered sufficient to provide descriptive information on the safety, tolerability, and preliminary efficacy of GBR830.

### Statistical Methods

The proportion of subjects who achieved an IGA score of 0 (clear) or 1 (almost clear) were summarized at Day 29 and Day 71, as were EASI50 response (250% reduction from baseline in EASI score) and EASI75 response (≥75% reduction). Percent improvements from baseline to Day 29 and Day 71 were summarized for

SCORAD and BSA affected. Mean changes from baseline to Week 4 and Week 10 in pruritus NRS score were also analyzed. All safety and clinical outcomes were analyzed descriptively.

### Demographics and Baseline Characteristics

64 eligible AD subjects were randomized in a 3:1 ratio to receive either GBR830 or corresponding placebo. Two subjects assigned to GBR830 withdrew informed consent prior to dosing. Therefore, only 62 subjects received treatment: GBR830, n=46; placebo, n=16 (ITT; FIG. 2). 40 AD subjects in the ITT population completed treatment and provided at least 2 biopsies: one at Day 1 before treatment (baseline) and at least another one after treatment (Day 29 or 71). Those 40 subjects who received two treatments and provided a biopsy (before and once after treatment) formed the Biological Activity Set (BAS: GBR830, n=29; placebo, n=11). Demographic characteristics were generally similar between treatment groups in both the ITT and the BAS population (Table 4). Analysis of baseline disease characteristics showed similarity between placebo and GBR830 subjects for EASI, SCORAD, and NRS. GBR830 subjects had higher baseline DIQI and IGA scores relative to placebo subjects (Table 4).

TABLE 4
Subject demographics and baseline disease characteristics (ITT and BAS population)

| | ITT | | BAS | | | | | |
|---|---|---|---|---|---|---|---|---|
| | GBR830 | | Placebo | | GBR830 | | Placebo | |
| | (n = 46) | | (n = 16) | | (n = 29) | | (n = 11) | |
| Demographics | | | | | | | | |
| Age, years | | | | | | | | |
| Mean ± SD | 36.2 ± 13.4 | | 40.4 ± 15.1 | | 34.1 ± 12.2 | | 40.7 ± 14.7 | |
| Median (min, max) | 34 | (18, 66) | 41 | (19, 59) | 33 | (18, 61) | 42 | (19, 59) |
| Sex, no. (%) | | | | | | | | |
| Male | 21 | (45.7) | 11 | (68.8) | 16 | (55.2) | 8 | (72.7) |
| Female | 25 | (54.3) | 5 | (31.2) | 13 | (44.8) | 3 | (27.3) |
| Race, no. (%) | | | | | | | | |
| Asian | 5 | (10.9) | 2 | (12.5) | 4 | (13.8) | 2 | (18.2) |
| Black or African | 9 | (19.6) | 3 | (18.7) | 5 | (17.2) | 1 | (9.1) |
| American | | | | | | | | |
| White | 31 | (67.4) | 11 | (68.8) | 19 | (65.5) | 8 | (72.7) |
| Other | 1 | (2.2) | 0 | | 1 | (3.4) | 0 | |
| Body mass index, | 26.1 ± 4.1 | | 26.2 ± 3.7 | | 25.7 ± 3.7 | | 26.1 ± 3.9 | |
| mean ± SD, kg/m² | | | | | | | | |
| Baseline disease | | | | | | | | |
| characteristics | | | | | | | | |
| BSA affected, mean | 38.6 ± 23.4 | | 39.3 ± 21.5 | | 38.6 ± 24.0 | | 38.4 ± 21.6 | |
| ± SD, % | | | | | | | | |
| SCORAD | | | | | | | | |
| Mean ± SD | 61.8 ± 14.3 | | 55.5 ± 10.4 | | 61.6 ± 15.8 | | 54.7 ± 10.8 | |
| Median (min, max) | 61.0 | (36.6, 94.4) | 56.4 | (37.8, 81.1) | 59.9 | (36.6, 94.4) | 54.0 | (37.8, 81.1) |
| EASI | | | | | | | | |
| Mean ± SD | 25.1 ± 12.3 | | 23.3 ± 9.4 | | 25.4 ± 13.7 | | 22.2 ± 9.6 | |
| Median (min, max) | 21.0 | (12.4, 65.0) | 19.9 | (14.1, 47.5) | 20.1 | (12.7, 65.0) | 18.9 | (14.1, 47.5) |
| DLQI, mean ± SD | 14.5 ± 6.7 | | 9.4 ± 5.7 | | 14.6 ± 6.9 | | 7.9 ± 5.1 | |
| IGA, no. (%) | | | | | | | | |
| Moderate | 26 | (56.5) | 11 | (68.8) | 16 | (55.2) | 8 | (72.3) |
| Severe/very severe | 20 | (43.5) | 5 | (31.2) | 13 | (44.8) | 3 | (27.3) |

| Epidermal thickness, | | | | | | |
|---|---|---|---|---|---|---|
| μm | | | | | | |
| Lesional | | | | | | |
| Mean ± SD | NA | NA | 140.6 ± 57.6 | | 125.0 ± 47.0 | |
| Median (min, max) | NA | NA | 130.2 | (58.4, 287.4) | 136.9 | (60.8, 187.1) |
| Non-iesional | | | | | | |
| Mean ± SD | NA | NA | 63.3 ± 25.2 | | 59.0 ± 21.5 | |
| Median (min, max) | NA | NA | 56.8 | (29.5, 155.6) | 54.2 | (33.1, 96.0) |

| | | | | |
|---|---|---|---|---|
| Pruritus NRS, mean | 6.4 ± 2.5 | 4.1 ± 2.6 | 6.3 ± 2.2 | 4.3 ± 1.9 |
| ± SD | | | | |

BAS, Biological Activity Set;
BSA, body surface area;
DLQI, Dermatology Life Quality Index;
EASI, Eczema Area and Severity Index;
IGA, investigator's global assessment (5-point scale);
ITT, intent-to-treat;
NA, not applicable;
NRS, numeric rating scale;
SCORAD, Scoring of Atopic Dermatitis;
SD, standard deviation.

### Safety

Safety and tolerability were assessed in all subjects who received at least one dose of treatment (ITT, n=62). 62.9% of subjects experienced at least 1 TEAE, with similar incidence between treatment groups during the 5 whole study period up to day 85 (Table 5). The most commonly reported TEAE was headache, with no clinically meaningful differences between GBR830 (13.0%) and placebo (25.0%). Most TEAEs were mild or moderate in intensity. Only 1 subject had a serious TEAE (coronary artery occlusion), which was not considered related to study treatment (GBR830) by the Investigator. No deaths occurred. No clinically meaningful differences in laboratory values, vital signs, physical examinations, and electrocardiograms were noted, and data were 10 similar between treatment groups. These data suggest that IV administration of GBR830 four weeks apart was safe and well tolerated in this proof-of-concept study.

TABLE 5
Treatment-emergent adverse events (ITT population)

| | GBR830 | | Placebo | |
|---|---|---|---|---|
| Adverse Events, n (%) | (n = 46) | | (n = 16) | |
| Deaths | 0 | | 0 | |
| Any TEAE | 29 | (63.0) | 10 | (63.0) |
| Any serious AE | 1 | (2.2) | 0 | |
| Discontinuation due to AEs | 2 | (4.3) | 1 | (6.3) |
| Common TEAEs | | | | |
| (≥5% of subjects in the GBR830 group) | | | | |
| Headache | 6 | (13.0) | 4 | (25.0) |
| Dermatitis atopic | 6 | (13.0) | 2 | (12.5) |
| Nasopharyngitis | 4 | (8.7) | 2 | (12.5) |
| Upper respiratory tract infection | 4 | (8.7) | 2 | (12.5) |

| | | | |
|---|---|---|---|
| Post-procedural infection | 4 | (8.7) | 0 |
| Myalgia | 3 | (6.5) | 0 |

AE, adverse event;
ITT, intent-to-treat;
TEAE, treatment-emergent adverse event.

### Biomarker Analyses

### GBR830 Hits Target and Reduces OX40 and OX40L in Lesional Skin

Changes with GBR830 in the direct target OX40, and its corresponding ligand OX40L, were assessed in pre- and post-treatment lesional skin. As seen with representative GBR830 and placebo subject images (FIG. 3, A and B), significant decreases from baseline in OX40⁺ T-cell and OX40L⁺ DC cellular staining in lesional skin were only found with GBR830 treatment at Day 29 (p<0.05) and Day 71 (p<0.001), with drug versus placebo trending on significance at Day 71 for both markers (FIG. 3, C and D). These data suggest that GBR830 successfully hits its target and inhibits the OX40/OX40L pathway.

### GBR830 Reduces Measures of Epidermal Hyperplasia and Proliferation

Changes in epidermal hyperplasia were measured via epidermal thickness of hematoxylin and eosin (H&E) sections (FIG. 4, A and D) and immunohistochemistry for two measures of epidermal proliferation, keratin 16 (K16; FIG. 4 B) and Ki67 (FIG. 4, C and F). K16 mRNA expression was evaluated using RT-PCR (FIG. 4 E). Representative subject images are shown in FIG. 4, A-C. Subjects treated with GBR830 demonstrated significant reductions from baseline in epidermal thickness at Day 29 (p<0.01) and Day 71 (p<0.001), with no significant changes in placebo-treated subjects (FIG. 4 D). K16 mRNA expression was significantly reduced from baseline at Days 29 and 71 (p<0.01 for both) in GBR830-treated subjects only (FIG. 4 E), with a significance difference between GBR830 and placebo at Day 71 (p<0.01). Larger, more significant reductions in Ki67⁺ cells were seen in GBR830-treated group, as compared to placebo at both Day 29 and Day 71 (p<0.001 In drug and p<0.05 in placebo; FIG. 4 F). Overall, the significant changes in measures of epidermal hyperplasia with drug suggest that GBR830 has beneficial effects on ameliorating the AD-associated epidermal pathology.

### OX40 Blockade Modulates Expression of Polar Immune Mediators

Expression of a selected number of immune genes previously linked with disease pathogenesis and/or upregulated in AD were measured via RT-PCR (FIG. 5, FIG. 6). The Th1 axis genes were significantly modulated only by GBR830, which induced downregulation of IFNγ at Day 29 (trend toward significance) and 71 (p<0.01 versus baseline), while upregulations were seen in placebo (FIG. 5A; p<0.01 at Day 71 for drug versus placebo). GBR830 also significantly modulated the IFNγ-induced chemokine CXCL10 at Days 29 and 71 (p<0.05 and p<0.001 versus baseline, respectively), with results approaching significance for GBR830 compared to placebo at Day 71 (FIG. 5 B).

Additionally, GBR830 induced progressive, significant downregulation at both days (with p<0.001 at Day 71) of several key Th2 markers including IL-31 and Th2-attracting chemokines, (CCL11, CCL17, and TSLPR; FIG. 5C-F), with significance achieved versus placebo (p<0.05) in CCL11 at Day 71 (FIG. 5 D) and in CCL17 at Day 29 (FIG. 5 E), with a trend toward significance versus placebo in CCL17 at Day 71 (FIG. 5 E). In fact, upregulations in these markers were seen with placebo at Day 29 and in CCL11 also at Day 71, with smaller and non-significant reductions in placebo-arm in IL-31 and CCL17 at Day 71. GBR830 also induced significant downregulations of Th17/Th22-related genes, including IL-23p19, IL-8, and S100As (FIG. 5 G-J). GBR830induced significant, progressive down-regulations towards Day 71 of these markers, with significance versus baseline (p<0.001) at both days for the IL-17/IL-22-regulated $100A9/S100A12 and at Day 71 for IL-23p19 and IL-8, with significance or trending towards significance versus placebo at both timepoints for all markers but IL-8 (FIG. 5 G-J). Of note, primary Th2 cytokines (IL-4 and IL-13) and Th17/Th22 cytokines (IL-17 and IL-22) were not significantly modulated with GBR830 compared with placebo (FIG. 6). Overall, significant and progressive differences were found between GBR830 and placebo treatments in several Th1 (IFN), Th2 (CCL11, CCL17), and Th17/Th22 measures (S100A12).

### Clinical Efficacy

The clinical effects of GBR830 were assessed in all subjects who received treatment (ITT population). Changes in disease activity over time (SCORAD, IGA, BSA, DLQI, NRS, and EASI) were analyzed by descriptive assessment during the treatment period of Day 1 up to Day 71. During this time, two doses of GBR830 or corresponding placebo were given on Day 1 and Day 29. As compared to baseline there was a gradual and continuous improvement in EASI score starting at Day 4 (FIG. 7A), including for subjects with severe disease at baseline, defined as having SCORAD>50 (FIG. 7 B). Interestingly, beginning with Day 15 there was a continuous and consistent separation between placebo and GBR830, showing a greater decline in EASI score for GBR830-treated subjects compared to placebo-treated subjects (FIG. 7A). This greater decline in EASI score first reached a maximum 22 days after the first dose (p<0.05), with a second maximum starting at Day 57 and maintained until Day 71 (p<0.05), the last treatment period day of taking skin biopsies. At Day 71, the percentage change from baseline indicated greater mean improvement with GBR830 relative to placebo (56% and 38%, respectively). EASI scores between GBR830 and placebo showed even greater separation through Day 71 in severe subjects (SCORAD>50) (FIG. 7 B). Subjects with the greatest improvement in EASI had the greatest improvement in IGA (data not shown).

Consistent with the improvement over time in EASI and SCORAD, categorical measurements of IGA showed greater improvement in GBR830-treated subjects compared to placebo (Table 6). The number of subjects with IGA response (score of 0 or 1) at Day 71 was 6/26 (23.1%) for GBR830 compared to 1/8 (12.5%) for placebo. Notably, the proportion of subjects with severe/very severe IGA at baseline was higher in the GBR830 group (20/46; 43.5%) compared to placebo (5/16; 31.2%). This finding is in line with the observation that EASI improvement in subjects with severe disease at baseline (SCORAD>50) may benefit more compared to the ITT population.

Higher proportions of GBR830-treated subjects achieved EASI50 and EASI75 compared to placebo (Table 6). EASI50 was achieved by 43.6% (17/39) of GBR830 and 20.0% (3/15) of placebo subjects at Day 29, and 76.9% (20/26) and 37.5% (3/8) of subjects at Day 71, respectively. EASI75 was achieved by 12.8% (5/39) of GBR830 and 6.7% (1/15) of placebo subjects at Day 29, and 42.3% (11/26) and 25.0% (2/8) of subjects at Day 71, respectively. Interestingly, all S subjects who achieved EASI75 at Day 29 maintained their improvement until Day 71, more than 42 days after their last dose (data not shown).

Changes is SCORAD, BSA, and pruritis NRS showed small numerical improvement with very high variability, making these data difficult to interpret in the ITT population. This variability was slightly lower in the BAS population. However, comparison of the efficacy endpoints between ITT and BAS population showed similarity in outcome measures between both populations (Table 6 and 7).

TABLE 6
Clinical endpoint outcomes (ITT population)

| | GBR830 | | Placebo | |
|---|---|---|---|---|
| Clinical Endpoint | (n = 46) | | (n = 16) | |
| IGA Response (score of 0 or 1), n/N (%) | | | | |
| Day 29 | 2/39 | (5.1) | 0 | |
| Day 71 | 6/26 | (23.1) | 1/8 | (12.5) |
| EASI50 (≥50% score reduction from baseline), n/N (%) | | | | |
| Day 29 | 17/39 | (43.6) | 3/15 | (20.0) |
| Day 71 | 20/26 | (76.9) | 3/8 | (37.5) |
| EASI75 (>75% score reduction from baseline), n/N (%) | | | | |
| Day 29 | 5/39 | (12.8) | 1/15 | (6.7) |
| Day 71 | 11/26 | (42.3) | 2/8 | (25.0) |

| | | |
|---|---|---|
| SCORAD, mean ± SD | | |
| Baseline | 62.2 ± 14.3 | 55.5 ± 10.4 |
| Day 29, % change from baseline | −28.2 ± 26.6 | −15.5 ± 21.7 |
| Day 71, % change from baseline | −45.4 ± 26.9 | −31.0 ± 16.9 |
| BSA affected, mean ± SD | | |
| Baseline | 38.6 ± 23.4 | 39.3 ± 21.5 |
| Day 29, % change from baseline | −28.5 ± 28.0 | −26.3 ± 30.7 |
| Day 71, % change from baseline | −47.1 ± 32.6 | −43.4 ± 34.9 |
| Pruritus NRS, mean ± SD | | |
| Baseline | 6.4 ± 2.2 | 4.9 ± 2.1 |
| Week 4, change from baseline | −1.6 ± 2.1 | −1.2 ± 1.6 |
| Week 10, change from baseline | −2.7 ± 2.5 | −1.5 ± 1.6 |

BSA, body surface area;
EASI, Eczema Area and Severity Index;
IGA, investigator's global assessment (5-point scale);
ITT, Intent-to-treat;
n, number of subjects who met response criterion;
N, number of subjects with non-missing values at the study visit;
NRS, numeric rating scale;
SCORAD, Scoring of Atopic Dermatitis;
SD, standard deviation.

TABLE 7
Clinical endpoint outcomes (BAS population)

| | GBR830 | | Placebo | |
|---|---|---|---|---|
| Clinical Endpoint | (n = 29) | | (n = 11) | |
| IGA Response (score of 0 or 1), n/N (%) | | | | |
| Day 29 | 2/29 | (6.9) | 0 | |
| Day 71 | 6/24 | (25.0) | 1/7 | (14.3) |
| EASI50 (≥50% score reduction | | | | |
| from baseline), n/N (%) | | | | |
| Day 29 | 15/29 | (51.7) | 3/11 | (27.3) |
| Day 71 | 18/24 | (75) | 3/7 | (42.9) |
| EASI75 (≥75% score reduction | | | | |
| from baseline), n/N (%) | | | | |
| Day 29 | 4/29 | (13.8) | 1/11 | (9.1) |
| Day 71 | 10/24 | (41.7) | 2/7 | (28.6) |

| | | |
|---|---|---|
| SCORAD, mean ± SD | | |
| Baseline | 61.5 ± 15.8 | 54.5 ± 10.8 |
| Day 29, % change from baseline | −30.5 ± 25.3 | −21.5 ± 17.4 |
| Day 71, % change from baseline | −44.0 ± 27.6 | −31.4 ± 18.2 |
| BSA affected, mean ± SD | | |
| Baseline | 38.6 ± 24.0 | 38.4 ± 21.6 |
| Day 29, % change from baseline | −32.2 ± 28.5 | −35.6 ± 30.9 |
| Day 71, % change from baseline | −45.6 ± 33.3 | −48.1 ± 34.7 |
| Pruritus NRS, mean ± SD | | |
| Baseline | 6.3 ± 2.2 | 4.3 ± 1.9 |
| Week 4, change from baseline | −2.0 ± 2.3 | −0.9 ± 1.4 |
| Week 10, change from baseline | −3.0 ± 2.6 | −1.3 ± 1.6 |

BAS, Biological Activity Set;
BSA, body surface area;
EASI, Eczema Area and Severity Index;
IGA, investigator's global assessment (5-point scale);
n, number of subjects who met response criterion;
N, number of subjects with non-missing values at the study visit;
NRS, numeric rating scale;
SCORAD, Scoring of Atopic Dermatitis;
SD, standard deviation.

### Conclusions

The present invention discloses the first clinical trial targeting a co-stimulatory molecule of immune regulation to treat moderate-to-severe AD patients. The present invention also provides the first evidence for the pathogenic role of the OX40 pathway in AD. The administration of two IV doses of the anti-OX40 antibody GBR830, 4 weeks apart, induced significant and progressive improvements in clinical severity scores and in the cutaneous molecular AD signature lasting until Day 71 (more than 42 days after the last dose). GBR830 was also well tolerated and showed an acceptable safety profile, with no clinically meaningful differences compared to placebo. While progressive clinical improvements (attaining significance at Day 71 compared to placebo) were observed with GBR830, we must remember that the study was not powered to detect clinical efficacy, but rather was designed primarily as a safety and mechanistic biomarker study.

The inhibition of OX40 pathway with GBR830 led to significant and progressive decreases in OX40⁺ T-cells as well as to changes in OX40L⁺ DCs that mark the “atopic DCs” in lesional skin. This indicates that GBR830 modulates the OX40-OX40L interaction, which is critical to the TSLP-mediated Th2 inflammation in atopic diseases. In addition to the modulation of key Th2 measures (IL-31, CCL11, CCL17, and TSLPR), OX40 antagonism also inhibited other immune pathways, which are also upregulated in AD, including Th1 (IFNγ, CXCL10) and Th17/Th22 (IL-23p19, IL-8, S100As). This effect may have particular value in AD, addressing the plasticity and diversity of disease endotypes. Furthermore, several subtypes, such as intrinsic, Asian, pediatric, and filaggrin⁺ AD subcategories were shown to have differential upregulations in Th17/Th22 or Th1 axes. Thus, Th1 and Th17/22 modulation in addition to Th2 may provide broader and/or more sustained therapeutic benefit. Interestingly, GBR830 did not have significant impact on mRNA expressions of the key Th2 cytokines (IL-4, IL-13), similar to dupilumab, and also did not show differential effects compared to placebo on the IL-22 and IL-17 cytokines. The observed clinical improvement was accompanied by significant improvements persisting up to Day 71 in hyperplasia measures (K16, Ki67, epidermal thickness) in lesional skin of GBR830-treated AD subjects, consistent with previous studies showing that clinical reversal by effective therapeutic intervention is also associated with improvement in the pathological epidermal hyperplasia characterizing active AD lesions.

The results of this study demonstrate for the first time in a clinical setting that targeting OX40 leads to downregulation of members of both the Th1 and Th2 dysregulated pathways and that these effects are associated with clinical improvement. Involvement of OX40-OX40L interaction has been demonstrated in several inflammatory conditions associated with atopy (i.e., asthma, allergic rhinitis, and allergic conjunctivitis). In preclinical in vivo mouse and non-human primate models of skin inflammation and asthma, blockade of OX401. significantly reduced the extent of Th2-mediated inflammation. Anti-OX40L antibodies also blocked TSLP-induced inflammation in the skin, as measured by decreased ear swelling responses and decreased cytokine mRNA expression levels (IL-4, IL-5, IL-13). In addition, inhibition of OX40-mediated signaling was also shown to reduce inflammation and ameliorate the severity of autoimmunity in pre-clinical models of multiple sclerosis (experimental autoimmune encephalomyelitis), asthma, and arthritis. In other diseases with a Th2 component, such as ulcerative colitis, OX40-OX40L has also been demonstrated to be increased and is now subject to clinical testing in ulcerative colitis patients (NCT02985593, NCT02647866).

In sum, targeting key switches of immune regulation, such as OX40, which regulates aberrant immune responses, may provide a novel therapeutic strategy for AD. Two doses of 4-weeks apart intravenous GBR830 were safe, well-tolerated, and induced significant and progressive clinical improvements, paralleled by molecular and cellular effects until Day 71 (more than 42 days post treatment). GBR830 may potentially provide a novel therapeutic paradigm for patients with moderate-to-severe AD, as it may induce durable disease control, ultimately reducing the frequency of drug administrations, perhaps similar to the IL-23-targeting strategies in psoriasis. This Invention showing that both the clinical and tissue disease pathology can be improved in an inflammatory human disease via OX40-targeting, coupled with the preclinical data of amelioration of inflammation through OX40/OX40L inhibition, has extended implications far beyond the skin, to other atopic or autoimmune conditions.

### Additional Data

Further experiments have been performed on the groups indicated in Table 8, see FIG. 8 to FIG. 11.

TABLE 8
Baseline patient characteristics in treatment and placebo groups

| | | Treatment | Placebo | |
|---|---|---|---|---|
| Characteristic | Parameter | Group | Group | p-value |
| Sex | Male | 21 | 11 | 0.15 |
| | Female | 25 | 5 | |
| Age | Total Range: 18-66 | 36.23 | 40.43 | 0.335 |
| | Asian | 5 | 2 | |
| Race | Black or African American | 9 | 3 | 1 |
| | Other | 1 | 0 | |
| | White | 31 | 10 | |
| Severity | SCORAD | 61.5 | 55.5 | 0.071 |
| Scores | EASI | 25.1 | 23.3 | 0.548 |
| H&E | Baseline, Non-Lesional | 60.39 | 56.13 | 0.556 |
| Thickness | Baseline, Lesional | 145.05 | 133.07 | 0.489 |

Results of the gene expression changes are presented in FIG. 12-FIG. 18. Results of Immunohistochemistry experiments are shown in FIG. 19-FIG. 28. Responders subanalysis (RT-PCR data) are shown in FIG. 29-FIG. 34 for Th17/Th22 related cytokines, in FIG. 35-FIG. 40 for Th2 specific chemokines, in FIG. 41-FIG. 42 for Th2 specific Cytokines, in FIG. 43-FIG. 47 for Th1/IFN related immune mediators, in FIG. 48-FIG. 49 for IHC data, in FIG. 50-FIG. 54 for Hyperplasia markers, and in FIG. 55-FIG. 57 are shown correlation heatmaps.

### Example 3: Biomarker Statistical Analysis

### Executive Summary

Biomarker were analyzed in blood and skin samples obtained from subjects enrolled in GBR830-201. Protein, mRNA and epigenetic analysis technologies were employed. Biomarker analysis of GBR830-201 provides evidence for GBR830 target engagement:

The expression of OX40, the pharmacologic target of GBR 830, was found to be reduced at visit 7 (treatment day 29) and 13 (treatment day 71). At visit 13, the expression change from baseline became nominally significant.

Reduction in TRAF2, TBK1, TANK, integral parts of Ox40/TNF-R pathway, was also found to be consistent with target engagement and provide evidence for the functional blockade of OX40. Interestingly, TRAF2 was downregulated only on the protein but not mRNA level and a regulation on the post-transcriptional level is consistent with published literature.

Furthermore a potential surrogate marker was identified as IFI27 gene expression sharply correlated with OX40 expression.

In a next step GBR830 mediated modulation of markers that relate to the pathogenesis of Atopic dermatitis were investigated:

While the acute phase of Atopic dermatitis is believed to be driven by Th2 cytokines, Th1 mediators are upregulated in the chronic phase of AD. GBR830 showed trends to suppress Th1 T cell derived cytokines such as IFNG and Th1 pathway biomarker CXCL9, CXCL10.

For Th2 pathway GBR 830 showed no trends to reduce in IL4. CCL11 showed trends to reduce at visit 13 on mRNA but not protein level. CCL17 showed trends to reduce at visit 13 for both NanoString and RT-PCR, however, same trends observed in the Placebo arm too.

KI67 which is found elevated in the skin of Atopic dermatitis patients due to the elevated proliferation of keratinocytes was found to be downregulated by GBR830, likely consistent with reduce epidermal proliferation and reduced epidermal thickening upon GBR830 treatment.

Biomarker for other pathways that are currently under evaluation for the treatment of Atopic dermatitis (e.g. IL31R, IL33R, TSLPR) did not show modulation by GBR 830.

Other biomarker that were found downregulated by GBR830 include BLNK, a B cell adapter protein and SMAD2, a component of TGFB signaling pathway. Interestingly TGFB/SMAD2 are recognized drivers of fibrotic processes and fibrosis Is also a recognized pathomechanistic pillar in chronic Atopic dermatitis.

### Biomarker Study Objectives

### PD Analysis Objectives

The PD analysis objectives of this biomarker study were:
Evaluate change from baseline in biomarkers at visit 7 and visit 13 by treatment arm
Evaluate change from baseline in biomarkers at visit 7 and visit 13 by response status in GBR 830 arm
Evaluate correlation between flow cytometry and Epiontis for the same cellular markers
Evaluate correlation between gene expression via NanoString and flow cytometry/Epiontis

### Clinical Endpoint Analysis Objectives

The clinical endpoint analysis objectives of this biomarker study were:
Evaluate association of baseline biomarker levels with EASI 75 response status at visit 7 and visit 13 respectively.
Evaluate association of baseline biomarker levels with EASI percentage change at visit 7 and visit 13 respectively
Evaluate association of baseline biomarker levels with EASI subscore at visit 7 and visit 13 respectively

### Clinical Study Design

### Overview of Clinical Study Design

This study was a Phase IIa, double-blind, randomized, Placebo-controlled, exploratory study to evaluate the safety, biological activity, and pharmacokinetics of GBR 830 In adult patients with moderate-to-severe atopic dermatitis (AD). The main objective of this study was to evaluate the effect of repeated doses of GBR 830 on biomarkers of disease activity in adult patients with moderate to severe AD. The objectives were exploratory in nature to further understand the mechanism of GBR 830 with the help of biomarker data. Placebo control was included to provide internal validity for the clinical trial and improved the sensitivity of the clinical trial for drug related changes and hence suited for an exploratory study.

### Study Treatments

In this study, the treatment was GBR 830. Subjects were randomized to the study drug, GBR 830 or Placebo in a 3:1 ratio.

### Design

Patients who meet eligibility criteria were to undergo Baseline (Day 1) assessments, randomization, and then receive the first IV infusion of GBR 830 or Placebo. Each patient received two doses of GBR 830 or Placebo, administered 4 weeks apart on Baseline (Day 1) and Visit 7 (Day 29). Patients were closely monitored at the study site for 6 hours after the first infusion on Day 1 and for 3 hours after the next dose on Visit 7 (Day 29). Patients returned for follow-up visits. Study sites contacted patients by telephone approximately 24 hours after each infusion (Day 2 and Day 30) to collect concomitant medication and procedure data, and a general AE query.

### During Treatment

The treatment phase consisted of the 2 visits (Day 1 and Day 29) which correspond to the study drug dosing days. Study drug IV infusions were to be administered on these days. Apart from the dosing visits, patients were seen in the clinic on Day 4, 8, 15, 22, 29, 32, 36, 43, 50, 57, 71 and the end of study visit, which occurs on Day 85 (week 12), for study assessments and PK sample collection. There was no extension phase planned for this study.

### Biomarker Study Design

### General Considerations

Patients were undergo baseline biopsy on Day 1 (pre-dose), repeat biopsy on Day 29 (pre-dose) and Day 71 (Visit 13).

There will be some degree of missing results due to sample availability in different biomarker assays.

TABLE 9
Sample Size by Visit

| Biomarker | Sample | | | Visit 7 | Visit 13 |
|---|---|---|---|---|---|
| Source | Matrix | Arm | Baseline | (Day 29) | (Day 71) |
| OLINK | Serum | GBR 830 | 28 | 27 | 25 |
| | | Placebo | 11 | 11 | 11 |
| RTPCR | RNA (Skin) | GBR 830 | 29 | 28 | 22 |
| | | Placebo | 11 | 11 | 7 |
| IHC | RNA (skin) | GBR 830 | 29 | 29 | 20 |
| | | Placebo | 11 | 10 | 7 |
| NanoString | Skin | GBR 830 | 23 | 23 | 18 |
| | | Placebo | 10 | 10 | 7 |
| Epiontis | Blood | GBR 830 | 23 | 23 | 20 |
| | | Placebo | 9 | 8 | 7 |
| Flow | Blood | GBR 830 | 25 | 23 | 20 |
| | | Placebo | 9 | 8 | 7 |
| ELISA | Blood | GBR 830 | 24 | 24 | 20 |
| | | Placebo | 9 | 9 | 7 |
| Epiontis | Blood | GBR 830 | 23 | 23 | 20 |
| | | Placebo | 9 | 8 | 7 |
| Flow | Blood | GBR 830 | 25 | 23 | 20 |
| | | Placebo | 9 | 8 | 7 |
| ELISA | Blood | GBR 830 | 24 | 24 | 20 |
| | | Placebo | 9 | 9 | 7 |

### Biomarker Analysis Sets

The following analysis sets were considered for biomarker efficacy evaluations:
Biomarker Analysis Set (BAS): subjects in the evaluable patient set as defined in the clinical protocol that have at least one biomarker sample at baseline.
Efficacy Analysis Set (EAS): BAS subjects who used prohibited medication, any clinical score on or after the date of first use of prohibited medication was set to missing.

### Biomarker Variables for PD Analysis

Flow Cytometry—cell count data—continuous varlabie
Flow Cytometry—cell percentage (of parent) data—continuous variable
NanoString gene expression data—continuous variable
Epiontis data—cell count data—continuous variable

### Biomarker Value Imputation

If biomarker data were missing due to LLOD or LLOQ, the missing values were imputed using half of LLOQ if appropriate.

Clinical Endpoints for Correlation of Biomarker Data with Efficacy

The EASI score is a validated measure used in clinical practice and clinical trials to assess the severity and extent of AD. Four AD disease characteristics were assessed for severity by the investigator or designee on a scale of “0” (absent) through “3” (severe). The biomarker analyses utilized the ADAM dataset used for the clinical analyses to ensure the same definitions of events for the various efficacy endpoints were being used. For correlation of biomarkers with clinical response, EASI-75 response was utilized. EASI subscores were analyzed in additional analyses.

### Statistical Methods

### General Considerations

Evaluations were performed based on the BAS. Due to the exploratory nature of this biomarker study, multiplicity adjustment was performed for the total number of statistical tests performed by biomarker type by objective. Multiplicity adjustment was performed to control the family-wise error rate (FWER) at 0.05 using Holm's approach (Holm, 1979).

### Subject Disposition

Subject disposition was summarized for each treatment arm and in total for all BAS subjects. The following subject disposition categories were included:
Subjects who were in the Biomarker Analysis Set (BAS)
Subjects who were in the Efficacy Analysis Set (EAS)

### Handling of Biomarker Data

### Overview of the Biomarker Data

### Flow Cytometry (17-Color Flow T-Regulatory/T-Helperpanel)

Precision's 17-color T-regulatory/T-helper panel was used to evaluate presence of cell populations of interest.

### Epiontis ID Data

Epiontis ID was based on cell type-specific, epigenetic biomarkers. These genomic biomarker regions were marked by the absence of CpG methylation in the respective cell types of interest, while all other cell types show complete methylation. Only demethylated biomarker regions reacted with bisulfite, a chemical used in the assay. Real time PCR was then employed to quantify the number of demethylated biomarker regions, and thus the precise number of the cell type of interest, in a wide range of sample matrices including whole blood, PBMCs, tissue or in isolated genomic DNA.

Five Epiontis assays used in this study included: Regulatory T cells, Overall T cells, Tfh Cells, TH17 cells and CD4 T cells.

### NonoString Gene Expression Data

The PanCancer Immune Panel enhanced with 14 gene custom code set was used.

PanCancer Immune Panel perform multiplex gene expression analysis with 770 genes from 24 different immune cell types, common checkpoint inhibitors, CT antigens, and genes covering both the adaptive and innate immune response. FIG. 58 showed brief descriptions of the immune cell types and selected genes included in the PanCancer Immune Profiling Panel. 14 additional genes were added to NanoString PanCancer Immune Panel, and they were: CD3, K16, ki67, MBP, BLIMP-1, Elafin/PI3, Filaggrin, hBD2, IL-23p40, IL-31, Loricrin, S100A9, TSLPR.

### Quality Control

### Flow Cytometry

Any percent parent frequency that were based on less than 50 cells in the parent population will be discarded from the data.

### Epiontis

The procedures for QC of Epiontis data were available in the corresponding product specifications “QMF 510-3e Product Information Sheet_Rev03 confidential.pdf”.

### NanoString

NanoString assay QC had two major metrics: binding density and Field of View (FOV) ratio. These two metrics could be retrieved in the Reporter Code Count (RCC) file generated by the NanoString nCounter digital analyzer. The binding density measures the number of optical features per square micron. Normally, the binding density was in the range of 0.05 to 2.25. If it was out of this range, the sample was flagged as failing to pass the imaging QC. FOV may indicate multiple issues during the imaging procedure. The NanoString digital analyzer reported the FOV counted which is the number of FOVs successfully imaged. If the ratio of FOV counted to FOV count (the number of FOVs attempted) was low, it might be indicative of an imaging issue. In this study, a sample will be flagged if its FOV ratio was less than 0.75. After QC, NanoString was normalized by housekeeping genes after determining the most stable set among housekeeping gene candidates.

### PD Analyses

### Impact of Drug Treatment on PD Profiles

Paired t-test was performed after biomarker data normalization comparing post-treatment (Visit 7 and Visit 13 respectively) and baseline by treatment group for all the biomarkers.

For the biomarker of interests, Mixed effect Model Repeat Measurement (MMRM) was perform to evaluate both post-treatment time biomarker expression change together, time effect by treatment arm and treatment effect were test by following MMRM model:

$${bmk}_{{cfbl}_{i}} = {bmk}_{{bl}_{i}} {β}_{{bmk}_{bl}} + {trt}_{i} {β}_{trt} + {vis}_{i} {β}_{vis} + {trt}_{i} * {vis}_{i} {β}_{trt * vis} + {X}_{i}^{T} {α}_{0} , \qquad ( 1 )$$

where for subject vv, bbbbkkccllii is biomarker baseline, bbbbkkccccccllii was biomarker expression change from baseline, ttttttii was the treatment effect and vvvvssii was the post-baseline visit time point (visit 7 and visit 13) and XXiiTT could be the covariates selected from demographic information or XXiiTT=0 when no covariates included in the model.

Covariance matrix will consider unstructured, but due to limited samples size and convergence issue, compound symmetry structure also was considered.

Contrast was constructed to evaluate treatment effect combining both post-baseline visits and corresponding p-value was reported.

Independent t-test was performed to biomarker data change from baseline comparing between EASI 75 response status in GBR-830 arm in visit 7 and visit 13 respectively.

For the biomarkers interests, least-squared means error bar figures or boxplots was generated to visualize the treatment effect on the biomarkers.

Biomarker expression considered in this analysis: normalized gene expression (NanoString), normalized cell counts data flow cytometry, and epigenetics (Epiontis).

Correlation between Flow Cytometry and Epigenetics

Evaluate both relative frequency and cell count correlation between flow cytometry and epigenetics in the same cellular marker by time point.

Pearson correlation coefficient (R) and corresponding p-value was reported. Visualization was generated for the cellular markers of interest.

Correlation between Gene Expression Data and Cell Count Data

Evaluated the correlation between the gene expression from NanoString and different cellular markers from flow cytometry and Epiontis by time point. Correlation between the gene expression changed from baseline and cellular markers changed from baseline was be evaluated.

### Biomarker Efficacy Analysis for EASI 75 Response

### Modeling Framework

Given the limited number of response in Placebo arm, only subjects in GBR-830 arm were considered. All analyses described in the following sections can be framed in the context of a generalized linear regression model (GLM) for the respective outcomes. For EASI 75 response outcomes the model was

$$logit ( p ( {Y}_{i} , {X}_{i} , {bmk}_{i} ) ) = f ( {bmk}_{i} ) {β}_{bmk} + {X}_{i}^{T} {α}_{0} \qquad ( 2 )$$

where for subject vv, YYii was the outcome of interest (response or not-response) in either visit 7 or visit 13, XXii was a vector of covariate values, bbbbkkii was baseline biomarker levels. The parameter ββbbbbkk represents the biomarker baseline effect and αα0 was a vector of parameters for any covariates included in the model.

Furthermore, ff(bbbbkkvv) was a function of biomarker that defines how biomarker was included in each model. Specifically, if biomarker was dichotomized at a cutoff c, then

$$f ( {bmk}_{i} ) = \{ \begin{matrix}1 & if {bmk}_{i} > c \\ 0 & if {bmk}_{i} \leq c\end{matrix} \qquad ( 3 )$$

where bmkᵢ=bmk_{%,i} or bmkᵢ=bmk_{cont,i} as indicated while

$$f ( {bmk}_{i} ) = {bmk}_{i} \qquad ( 4 )$$

if biomarker was a continuous variable treated as a continuous variable, or biomarker was a categorical variable treated as a categorical variable,

### Biomarker Effect

The EASI 75 response at visit 7 and visit 13 for biomarker effect analysis of this study evaluated respectively whether patients with certain biomarker characteristics at baseline show statistically significant difference on response status. This equated to the following hypotheses:

$${H}_{0} : {β}_{bmk} = 0 vs . {H}_{a} : {β}_{bmk} \neq 0 \qquad ( 3 )$$

(3) evaluated the biomarker effect. The hypothesis will be tested using the logistic model outlined in Equation (2). Likelihood ratio test was used for (3). Statistical significance in favor of rejecting the null hypothesis at an alpha level of 0.05 if the adjusted p-value for the test outlined in hypothesis (10) or (11) derived from the logistic regression model was less than 0.05. For categorical biomarker: the probability of getting response by the biomarker status along with the 95% CI was presented. Additionally, Odds Ratio (OR) comparing between biomarker statuses was presented.

### Programming Considerations

All tables, data listings, figures (TLFs), and statistical analyses was generated using SAS* Version 9.4 or higher or R Version 3.4 or higher.

### PD Analysis Results

### Results Summary for GBR 830 Targets

GBR 830 is a novel, antagonistic monoclonal antibody that was designed to selectively target OX40 receptors to reduce inflammation in atopic dermatitis. In visit 13 (Day71), there was a nominal significant reduction for both OX40 in the dermis and OX40 in the epidermis comparing to the baseline in GBR 830 arm. (FIG. 59 and FIG. 60). Correlation between IFI27 and OX40 expression (FIG. 61) showed potential new insights into OX40 mechanism and link to Type I interferon pathway. IFI27 expression showed a nominal significant change from baseline at visit 13. Table 10 shows overall results for these three biomarkers

TABLE 10
Drug Targeted Biomarkers Result from PD Analysis

| | | | Visit 7 | | | | Visit 13 | | | |
|---|---|---|---|---|---|---|---|---|---|---|
| | | | | Change | | p-value | | Change | | p-value |
| | | | | from | | GBR vs | | from | | GBR vs |
| Source | Biomarker | Arm | n | Baseline | p-value | PBO | n | Baseline | p-value | PBO |
| IHC | OX40.DERMIS | GBR | 29 | −0.335 | 0.4962 | 0.8282 | 20 | −1.644 | 0.0060 | 0.2470 |
| | | PBO | 10 | −0.805 | 0.3367 | | 7 | −0.321 | 0.7430 | |
| | OX40.EPIDERMIS | GBR | 29 | −0.326 | 0.3347 | 0.7439 | 20 | −0.874 | 0.0274 | 0.3997 |
| | | PBO | 10 | −0.109 | 0.8480 | | 7 | −0.233 | 0.7217 | |
| NanoString | IFI27 | GBR | 23 | −0.377 | 0.0458 | 0.5989 | 19 | −0.547 | 0.0090 | 0.2977 |
| | | PBO | 11 | −0.205 | 0.4464 | | 8 | −0.155 | 0.6205 | |

### Results Summary for Biomarkers in YNF-R Pathway

TNF receptors engage multiple signaling and adaptor proteins such as TRAF2 and TANK to activate and other kinases and NFkB is crucial for the induction of inflammatory mediators. Reduction in TRAF2, TANK was shown consistent with GBR830 target engagement and blockade of OX40 from OLINK using biopsy sample (FIG. 63 and FIG. 64). TANK expression from NanoString (FIG. 66) showed continuous reduction at visit 13, while TRAF2 expression from NanoString (FIG. 65) did not show a significant change from baseline.

TBK1 (FIG. 67) showed a significant reduction at visit 13, and suppression of TBK1 (TANK Binding Kinase 1) linked to augmented T-reg numbers and function. Table 11 shows more detailed information.

TABLE 11
Table 8.2-1 Results for TNF-R Pathway Biomarkers

| | | | Visit 7 | | | | Visit 13 | | | |
|---|---|---|---|---|---|---|---|---|---|---|
| | | | | Change | | p-value | | Change | | p-value |
| | | | | from | | GBR vs | | from | | GBR vs |
| Source | Biomarker | Arm | n | Baseline | p-value | PBO | n | Baseline | p-value | PBO |
| NanoString | TANK | GBR | 23 | −0.046 | 0.3210 | 0.4445 | 19 | −0.182 | 7e−04 | 0.0271 |
| | | PBO | 11 | 0.016 | 0.8077 | | 8 | 0.031 | 0.6969 | |
| | TBK1 | GBR | 23 | −0.002 | 0.9818 | 0.8196 | 19 | −0.311 | 0.0034 | 0.1223 |
| | | PBO | 11 | −0.040 | 0.7696 | | 8 | −0.017 | 0.9140 | |
| | TRAF2 | GBR | 23 | −0.025 | 0.7049 | 0.6237 | 19 | 0.041 | 0.5786 | 0.7526 |
| | | PBO | 11 | 0.033 | 0.7375 | | 8 | −0.002 | 0.9877 | |
| OLINK | TANK | GBR | 27 | −0.068 | 4.5e−05 | 0.6709 | 25 | −0.048 | 0.0045 | 0.8265 |
| | | PBO | 11 | −0.080 | 0.0017 | | 11 | −0.054 | 0.0310 | |
| | TRAF2 | GBR | 27 | −0.273 | 4.7e−06 | 0.0629 | 25 | −0.320 | 3.7e−07 | 0.0372 |
| | | PBO | 11 | −0.069 | 0.4447 | | 11 | −0.087 | 0.3329 | |

### Results Summary for Biomarkers In Th1 Pathway

IFNγγ and Th1 cytokines are upregulated in the chronic phase of AD. GBR 830 showed trends to suppress IFNγγ signature biomarkers such as CXCL9 and CXCL10 from NanoString (FIG. 68 and FIG. 69), CXCL10 from OLINK (FIG. 72), and CXCL10 from RT-PCR (FIG. 74). IFNγγ (FIG. 75) showed reduction trend over time In GBR 830 arm, but the change was not significant. Table 12 shows detailed results for CXCL9, CXCL10, and CXCL11.

TABLE 12
Th1 pathway Biomarkers Result from PD Analysis

| | | | Visit 7 | | | | Visit 13 | | | |
|---|---|---|---|---|---|---|---|---|---|---|
| | | | | Change | | p-value | | Change | | p-value |
| | | | | from | | GBR vs | | from | | GBR vs |
| Source | Biomarker | Arm | n | Baseline | p-value | PBO | n | Baseline | p-value | PBO |
| NanoString | CXCL9 | GBR | 23 | −0.271 | 0.4687 | 0.9826 | 19 | −1.540 | 4.2e−04 | 0.1372 |
| | | PBO | 11 | −0.257 | 0.6371 | | 8 | −0.390 | 0.5413 | |
| | CXCL10 | GBR | 23 | −0.699 | 0.1940 | 0.5577 | 19 | −2.228 | 3.3e−04 | 0.2573 |
| | | PBO | 11 | −0.145 | 0.8515 | | 8 | −1.001 | 0.2700 | |
| | CXCL11 | GBR | 23 | −1.000 | 0.0325 | 0.3617 | 19 | −1.437 | 0.0058 | 0.5495 |
| | | PBO | 11 | −0.262 | 0.8925 | | 8 | −1.991 | 0.0126 | |
| OLINK | CXCL9 | GBR | 27 | 0.062 | 0.8638 | 0.9081 | 25 | −0.104 | 0.4804 | 0.3074 |
| | | PBO | 11 | 0.031 | 0.8896 | | 11 | 0.172 | 0.4462 | |
| | CXCL10 | GBR | 27 | −0.034 | 0.7871 | 0.2895 | 25 | −0.340 | 0.0116 | 0.0324 |
| | | PBO | 11 | 0.218 | 0.2769 | | 11 | 0.183 | 0.3615 | |
| | CXCL11 | GBR | 27 | 0.170 | 0.1623 | 0.5609 | 25 | −0.012 | 0.9213 | 0.6304 |
| | | PBO | 11 | 0.039 | 0.8385 | | 11 | 0.097 | 0.8093 | |
| RT-PCR | CXCL10 | GBR | 27 | −0.516 | 0.1365 | 0.3383 | 22 | −1.280 | 0.0012 | 0.2859 |
| | | PBO | 11 | 0.098 | 0.8560 | | 7 | −0.454 | 0.4999 | |
| | IFNg | GBR | 27 | −0.520 | 0.1583 | 0.2590 | 22 | −0.669 | 0.1033 | 0.0693 |
| | | PBO | 11 | 0.258 | 0.6552 | | 7 | 0.877 | 0.2300 | |

### Results Summary for Biomarkers In Th2 Pathway

GBR 830 showed no trends to reduce in IL4 in NanoString or OLINK (FIG. 76 and FIG. 77). CCL11 showed trends to reduce at visit 13 for RT-PCR (FIG. 80), but no In NanoString or OLINK (FIG. 78 and FIG. 79). CCL17 showed trends to reduce at visit 13 for both NanoString and RT-PCR, however, same trends observed in the Placebo arm too. (FIG. 81 and FIG. 82)

TABLE 13
Th2 pathway Biomarkers Result from PD Analysis

| | | | Visit 7 | | | | Visit 13 | | | |
|---|---|---|---|---|---|---|---|---|---|---|
| | | | | Change | | p-value | | Change | | p-value |
| | | | | from | | GBR vs | | from | | GBR vs |
| Source | Biomarker | Arm | n | Baseline | p-value | PBO | n | Baseline | p-value | PBO |
| NanoString | CCL11 | GBR | 23 | −0.055 | 0.8503 | 0.4952 | 19 | −0.434 | 0.1786 | 0.2005 |
| | | PBO | 11 | −0.416 | 0.3368 | | 8 | 0.335 | 0.5014 | |
| | CCL17 | GBR | 23 | 0.277 | 0.5766 | 0.1067 | 19 | −1.269 | 0.0225 | 0.7077 |
| | | PBO | 11 | −1.144 | 0.1133 | | 8 | −1.644 | 0.0540 | |
| | IL4 | GBR | 23 | 0.284 | 0.0328 | 0.2271 | 19 | 0.032 | 0.8098 | 0.7311 |
| | | PBO | 11 | 0.002 | 0.9893 | | 8 | −0.052 | 0.7985 | |
| OLINK | CCL11 | GBR | 27 | −0.007 | 0.8946 | 0.3561 | 25 | −0.014 | 0.7832 | 0.2044 |
| | | PBO | 11 | 0.082 | 0.3115 | | 11 | 0.108 | 0.1806 | |
| | IL-4 | GBR | 27 | −0.005 | 0.5070 | 0.8464 | 25 | −0.008 | 0.3177 | 0.0418 |
| | | PBO | 11 | −0.002 | 0.8447 | | 11 | 0.023 | 0.0736 | |
| RT-PCR | CCL11 | GBR | 27 | 0.013 | 0.9761 | 0.6362 | 22 | −0.950 | 0.0458 | 0.2312 |
| | | PBO | 11 | 0.389 | 0.5598 | | 7 | 0.212 | 0.8003 | |
| | CCL17 | GBR | 27 | −0.393 | 0.2970 | 0.6147 | 22 | −1.058 | 0.0112 | 0.8186 |
| | | PBO | 11 | −0.033 | 0.9562 | | 7 | −0.867 | 0.2279 | |
| | IL-4 | GBR | 27 | 0.211 | 0.4906 | 0.4499 | 22 | −0.182 | 0.5879 | 0.3174 |
| | | PBO | 11 | 0.640 | 0.1839 | | 7 | −0.865 | 0.1472 | |

### Existing or Emerging AD Treatments Pharmacologic Target Results Summary

IL31RA from target of Nemolizumab, TSLPR and IL33R (IL1RL1) did not show reduction in GBR 830 arms (FIG. 83-FIG. 87), Table 14 shows more detailed information.

TABLE 14
Existing or emerging AD treatments pharmacologic target results summary

| | | | Visit 7 | | | | Visit 13 | | | |
|---|---|---|---|---|---|---|---|---|---|---|
| | | | | Change | | p-value | | Change | | p-value |
| | | | | from | | GBR vs | | from | | GBR vs |
| Source | Blomarker | Arm | n | Baseline | p-value | PBO | n | Baseline | p-value | PBO |
| NanoString | IL-31 | GBR | 23 | 0.111 | 0.5900 | 0.1736 | 19 | −0.263 | 0.2475 | 0.7531 |
| | | PBO | 11 | −0.395 | 0.1924 | | 8 | −0.395 | 0.2630 | |
| | IL1RL1 | GBR | 23 | 0.115 | 0.4587 | 0.6923 | 19 | −0.146 | 0.3929 | 0.2711 |
| | | PBO | 11 | 0.223 | 0.3212 | | 8 | −0.495 | 0.0650 | |
| | TSLPR | GBR | 23 | 0.017 | 0.9388 | 0.9856 | 19 | −0.486 | 0.0493 | 0.6167 |
| | | PBO | 11 | 0.010 | 0.9753 | | 8 | −0.262 | 0.4872 | |
| RT-PCR | IL31 | GBR | 27 | 0.233 | 0.4204 | 0.9599 | 22 | −0.506 | 0.1115 | 0.6166 |
| | | PBO | 11 | 0.205 | 0.8529 | | 7 | −0.181 | 0.7451 | |
| | TSLPR | GBR | 27 | −0.069 | 0.5519 | 0.7767 | 22 | −0.217 | 0.0870 | 0.5247 |
| | | PBO | 11 | −0.131 | 0.4771 | | 7 | −0.380 | 0.0871 | |

### Results Summary for Other Biomarkers of Interests

Ki67, BLNK (related to B cell adapter protein), and SMAD2 were downregulated by GBR 830 (FIG. 88-FIG. 90). Table 15 shows additional results summaries.

TABLE 15
Other biomarkers of interest results summary

| | | | Visit 7 | | | | Visit 13 | | | |
|---|---|---|---|---|---|---|---|---|---|---|
| | | | | Change | | p-value | | Change | | p-value |
| | | | | from | | GBR vs | | from | | GBR vs |
| Source | Biomarker | Arm | n | Baseline | p-value | PBO | n | Baseline | p-value | PBO |
| IHC | Ki67.EPIDERMIS | GBR | 29 | −0.930 | 0.0035 | 0.1892 | 20 | −1.174 | 0.0020 | 0.2889 |
| | | PBO | 10 | −0.113 | 0.8294 | | 7 | −0.410 | 0.5076 | |
| NanoString | BLNK | GBR | 23 | −0.039 | 0.8343 | 0.5442 | 19 | −0.678 | 0.0014 | 0.0049 |
| | | PBO | 11 | 0.158 | 0.5526 | | 8 | 0.410 | 0.1939 | |
| | SMAD2 | GBR | 23 | 0.003 | 0.9128 | 0.9695 | 19 | −0.116 | 9e−04 | 0.1755 |
| | | PBO | 11 | 0.001 | 0.9769 | | 8 | −0.032 | 0.5298 | |

### Difference in PD Profiles in Responders in GBR 830 Arm

This analysis focused on responders in GBR 830 arm at visit 7 and visit 13 respectively. Only NanoString, Flow Cytometry, Elisa, Epiontis biomarkers were considered. Top results are listed in

### Results Summary for GBR 830 Targets

IFI27 were correlated with OX40, but it (FIG. 91) showed no significant changes from baseline among the responder in GBR 830 arm

TABLE 16
Drug targeted biomarker paired T-test post-baseline vs baseline among responder in GBR 830 arm

| | | Visit 7 | | | Visit 13 | | |
|---|---|---|---|---|---|---|---|
| | | change from | | | change from | | |
| Source | Biomarker | baseline | change CI | p-value | baseline | change CI | p-value |
| NanoString | IFI27 | −0.465 | (−2.283, 1.353) | 0.475 | −0.661 | (−1.639, 0.317) | 0.154 |

### Results Summary for Biomarkers in TNF-R Pathway

TBK1 shows marginal significant reduction in expression at visit 13 among responders (FIG. 92)

TABLE 17
TNF-R pathway biomarker paired T-test post-baseline vs baseline among responder in GBR 830 arm

| | | Visit 7 | | | Visit 13 | | |
|---|---|---|---|---|---|---|---|
| | | change from | | | change from | | |
| Source | Biomarker | baseline | change CI | p-value | baseline | change CI | p-value |
| NanoString | TANK | −0.213 | (−0.510, 0.083) | 0.106 | 0.080 | (−0.229, 0.069) | 0.245 |
| | TBK1 | −0.080 | (−0.462, 0.301) | 0.550 | −0.107 | (−0.233, 0.019) | 0.084 |
| | TRAF2 | −0.065 | (−0.478, 0.347) | 0.649 | −0.023 | (−0.292, 0.246) | 0.847 |

### Results Summary for Biomarkers in Th1 Pathway

CXCL9, CXCL10, CXCL11 from Th1 pathway showed marginal significant change from baseline among responders at visit 13 (FIG. 93), and trend could be observed at visit 7.

TABLE 18
Th1 pathway biomarker paired T-test post-baseline vs baseline among responder in GBR 830 arm

| | | Visit 7 | | | Visit 13 | | |
|---|---|---|---|---|---|---|---|
| | | change from | | | change from | | |
| Source | Biomarker | baseline | change CI | p-value | baseline | change CI | p-value |
| NanoString | CXCL9 | −1.194 | (−4.490, 2.102) | 0.333 | −1.524 | (−3.083, 0.034) | 0.054 |
| | CXCL10 | −1.759 | (−5.470, 1.952) | 0.228 | −3.002 | (−5.880, −0.125) | 0.043 |
| | CXCL11 | −2.353 | (−5.197, 0.491) | 0.078 | −1.639 | (−3.227, −0.052) | 0.045 |

### Results Summary for Biomarkers in Th2 Pathway

CCL11, CCL12 and IL4 from Th2 pathway showed no significant change from baseline among responders at visit 7 or visit 13 (FIG. 94).

TABLE 19
Th2 pathway biomarker paired T-test post-baseline
vs baseline among responder in GBR 830 arm

| | | Visit 7 | | | Visit 13 | | |
|---|---|---|---|---|---|---|---|
| | | change | | | change | | |
| | | from | | | from | | |
| Source | Biomarker | baseline | change CI | p-value | baseline | change CI | p-value |
| NanoString | CCL11 | 0.000 | — | | −0.234 | (−1.221, 0.752) | 0.592 |
| | CCL17 | −0.038 | (−7.552, 7.476) | 0.988 | −0.825 | (−3.518, 1.868) | 0.492 |
| | IL4 | −0.009 | (−0.037, 0.019) | 0.391 | 0.307 | (−0.198, 0.812) | 0.194 |

### Existing or Emerging AD Treatments Pharmacologic Target Results Summary

None of the biomarkers showed significant change from baseline among responders (FIG. 95).

TABLE 20
Existing or emerging AD treatments pharmacologic target biomarker paired
T-test post-baseline vs baseline among responder in GBR 830 arm

| | | Visit 7 | | | Visit 13 | | |
|---|---|---|---|---|---|---|---|
| | | change | | | change | | |
| | | from | | | from | | |
| Source | Biomarker | baseline | change CI | p-value | baseline | change CI | p-value |
| NanoString | IL-31 | 0.000 | — | | 0.050 | (−1.285, 1.384) | 0.932 |
| | IL1RL1 | −0.043 | (−0.941, 0.854) | 0.888 | −0.008 | (−0.509, 0.493) | 0.970 |
| | TSLPR | −0.401 | (−2.901, 2.099) | 0.645 | −0.495 | (−1.562, 0.571) | 0.309 |

### Results Summary for Other Biomarkers of Interests

None of the biomarkers showed significant change from baseline among responders (FIG. 96

TABLE 21
Other biomarkers of interest paired T-test post-
baseline vs baseline among responder in GBR 830 arm

| | | Visit 7 | | | Visit 13 | | |
|---|---|---|---|---|---|---|---|
| | | change | | | change | | |
| | | from | | | from | | |
| Source | Biomarker | baseline | change CI | p-value | baseline | change CI | p-value |
| NanoString | BLNK | −0.069 | (−0.524, 0.385) | 0.660 | −0.224 | (−0.764, 0.316) | 0.359 |
| | SMAD2 | −0.232 | (−0.535, 0.070) | 0.092 | −0.127 | (−0.312, 0.058) | 0.149 |

### Efficacy Endpoint Analysis Results

Efficacy Analysis Set (EAS) was used for all the efficacy endpoint analysis.

EASI 75 Change from Baseline in EASI Score, and EASI Subscores

MMRM was performed using all post-baseline time points by each endpoint with heterogeneous first order autoregressive (ARH(1)) covariance structure.

EASI scores decreased over time, and the difference between GBR 830 arm and Placebo arm Increased over time (FIG. 97). Visit 13 shows marginal significant different between GBR 830 arm and Placebo arm with p-value=0.011 (Table 22)

TABLE 22
MMRM result with selected post-baseline time points - EASI score

| | GBR830 | | | Placebo | | | |
|---|---|---|---|---|---|---|---|
| | Change from Baseline (%) | | | Change from Baseline (%) | | | |
| | | | Conf. | | | Conf. | p-value |
| Visit | n | estimates | Interval | n | estimates | Interval | GBR vs PBO |
| Visit 3 (day 4) | 40 | −10.651 | (−16.4, −4.9) | 14 | −11.186 | (−21.0, −1.4) | 0.925 |
| Visit 7 (day 29) | 34 | −34.026 | (−46.7, −21.3) | 13 | −30.113 | (−50.9, −9.3) | 0.749 |
| Visit 12 (day 57) | 25 | −59.077 | (−70.0, −48.2) | 10 | −41.208 | (−58.8, −23.6) | 0.090 |
| Visit 13 (day 71) | 23 | −63.728 | (−72.9, −54.6) | 8 | −40.276 | (−55.5, −25.0) | 0.011 |

Subscores of EASI represent four symptoms: Erythema, Induration/Papules, Excoriation, and Lichenification. Each subscore was calculated by severity scores from four body parts: head, trunk upper extremities, and lower extremities. Each severity score ranges from 0 (none) to 3 (severe). As a result, each subscore of EASI range from 0 to 12.

Excoriation (FIG. 98) represents chronic symptoms. This shows significant difference between treatment groups at Visit 12 and 13 (Table 23).

TABLE 23
MMRM result with selected post-baseline time points - Excoriation score

| | GBR830 | | | Placebo | | | |
|---|---|---|---|---|---|---|---|
| | Change from Baseline (%) | | | Change from Baseline (%) | | | |
| | | | Conf. | | | Conf. | p-value |
| Visit | n | estimates | Interval | n | estimates | Interval | GBR vs PBO |
| Visit 3 (day 4) | 40 | −9.744 | (−15.9, −3.6) | 14 | 1.482 | (−9.0, 12.0) | 0.073 |
| Visit 7 (day 29) | 34 | −30.576 | (−45.2, −15.9) | 13 | −21.638 | (−45.5, 2.2) | 0.526 |
| Visit 12 (day 57) | 25 | −61.402 | (−74.7, −48.1) | 10 | −34.145 | (−55.6, −12.7) | 0.035 |
| Visit 13 (day 71) | 23 | −64.286 | (−76.4, −52.2) | 8 | −37.921 | (−58.2, −17.6) | 0.030 |

Lichenification (FIG. 99) represents chronic symptoms. This shows a marginal significant difference between treatment groups at Visit 13 (Table 24).

TABLE 24
MMRM result with selected post-baseline time points - Lichenification score

| | GBR830 | | | Placebo | | | |
|---|---|---|---|---|---|---|---|
| | Change from Baseline (%) | | | Change from Baseline (%) | | | |
| | | | Conf. | | | Conf. | p-value |
| Visit | n | estimates | Interval | n | estimates | Interval | GBR vs PBO |
| Visit 3 (day 4) | 40 | −0.952 | (−7.9, 6.0) | 14 | 6.296 | (−5.5, 18.0) | 0.295 |
| Visit 7 (day 29) | 34 | −23.619 | (−36.4, −10.9) | 13 | −8.377 | (−29.2, 12.4) | 0.218 |
| Visit 12 (day 57) | 25 | −38.821 | (−52.4, −25.2) | 10 | −19.140 | (−41.2, 2.9) | 0.134 |
| Visit 13 (day 71) | 23 | −43.790 | (−54.8, −32.8) | 8 | −16.856 | (−35.3, 1.6) | 0.015 |

Induration (swelling) (FIG. 100) represents acute symptoms. This shows a marginal significant difference between treatment groups at visit 13.(Table 25)

TABLE 25
MMRM result with selected post-baseline time points - Induration score

| | GBR830 | | | Placebo | | | |
|---|---|---|---|---|---|---|---|
| | Change from Baseline (%) | | | Change from Baseline (%) | | | |
| | | | Conf. | | | Conf. | p-value |
| Visit | n | estimates | Interval | n | estimates | Interval | GBR vs PBO |
| Visit 3 (day 4) | 40 | −8.038 | (−13.4, −2.7) | 14 | −7.374 | (−16.4, 1.7) | 0.899 |
| Visit 7 (day 29) | 34 | −28.713 | (−39.5, −17.9) | 13 | −20.829 | (−38.4, −3.2) | 0.449 |
| Visit 12 (day 57) | 25 | −50.125 | (−60.8, −39.4) | 10 | −33.122 | (−50.4, −15.9) | 0.099 |
| Visit 13 (day 71) | 23 | −54.454 | (−64.2, −44.7) | 8 | −34.096 | (−50.4, −17.8) | 0.036 |

Erythema (FIG. 101) represents acute symptoms. This shows no significant difference between treatment groups across all time points.(Table 26)

TABLE 26
MMRM result with selected post-baseline time points - Erythema score

| | GBR830 | | | Placebo | | | |
|---|---|---|---|---|---|---|---|
| | Change from Baseline (%) | | | Change from Baseline (%) | | | |
| | | | Conf. | | | Conf. | p-value |
| Visit | n | estimates | Interval | n | estimates | Interval | GBR vs PBO |
| Visit 3 (day 4) | 40 | −5.975 | (−11.5, −4.1e−01) | 14 | −9.132 | (−18.6, 3.7e−01) | 0.568 |
| Visit 7 (day 29) | 34 | −30.243 | (−40.9, −19.6) | 13 | −21.722 | (−39.1, −4.3) | 0.408 |
| Visit 12 (day 57) | 25 | −45.939 | (−55.3, −36.6) | 10 | −41.277 | (−56.4, −26.2) | 0.603 |
| Visit 13 (day 71) | 23 | −46.883 | (−57.3, −36.5) | 8 | −34.129 | (−51.4, −16.8) | 0.211 |

### Biomarker at Baseline Dichotomization

Biomarker at Baseline values were dichotomized based on median of each biomarker from all patients. Biomarker high group was defined as expression of the blomarker greater than or equal to median expression; and low group otherwise.

Results of Biomarkers Correlated with EASI 75 Response

Due to the limited number of responders in the Placebo arm, this analysis was performed on the GBR 830 patients only. Because of limited number of patients in response or non-response in either biomarker high group or low group, Fisher's exact test was used to test the relationship between biomarker groups and response status.

Top results were selected based on raw p-value <0.1 at visit 13 (or visit 14 LOCF). The top biomarkers were ranked by the minimum of raw p-value from visit 7 (or visit 12 LOCF) and visit 13 (or visit 14 LOCF). Top five biomarker results showed in Table 27.

TABLE 27
Top five biomarker results related to EASI 75 response status

| | | | | Sample Size | | Response Rate | | Odds Ratios | |
|---|---|---|---|---|---|---|---|---|---|
| | | | | | Non- | | Conf. | Low vs | |
| Source | Biomarker | Visit | Group | Resp. | responder | Estimates | Interval | High | p-value |
| OLINK | BACH1 | Visit 12 | high | 7 | 4 | [symbol] | (0.354, 0.848) | 0.052 | 0.0072 |
| | | LOCF | low | 1 | 13 | 0.071 | (0.004, 0.315) | 0.052 | 0.0072 |
| | | Visit 14 | high | 6 | 5 | 0.545 | (0.28, 0.787) | 0.152 | 0.0810 |
| | | LOCF | low | 2 | 12 | 0.143 | (0.04, 0.399) | 0.152 | 0.0810 |
| | CD-83 | Visit 12 | high | 2 | 12 | 0.143 | (0.04, 0.399) | 6.579 | 0.0810 |
| | | LOCF | low | 6 | 5 | 0.545 | (0.28, 0.787) | 6.579 | 0.0810 |
| | | Visit 14 | high | 1 | 13 | 0.071 | [symbol] | 19.408 | 0.0072 |
| | | LOCF | low | 7 | 4 | [symbol] | (0.354, 0.848) | 19.408 | 0.0072 |
| Flow | CD4+ | Visi 7 | high | 0 | 13 | 0.000 | (0, 0.228) | Inf | [symbol] |
| | CCR10+ | | low | 3 | 7 | 0.300 | (0.108, 0.603) | Inf | [symbol] |
| | Th22 helper | Visit 13 | high | 1 | 8 | 0.111 | [symbol] | 17.540 | [symbol] |
| | cells | | low | 8 | 3 | 0.727 | [symbol] | 17.540 | [symbol] |
| NanoString | CD209 | Visit 7 | high | 0 | 10 | 0.000 | (0, 0.278) | Inf | 0.0867 |
| | | | low | 4 | [symbol] | 0.400 | [symbol] | Inf | 0.0867 |
| | | Visit 13 | high | 1 | 7 | 0.125 | (0.006, 0.471) | 31.607 | 0.0101 |
| | | | low | 7 | 1 | 0.875 | (0.529, 0.994) | 31.607 | 0.0101 |
| | SPN | Vielt 7 | high | 0 | [symbol] | 0.000 | (0, 0.299) | Inf | 0.0941 |
| | | | low | 4 | 7 | 0.364 | [symbol] | Inf | 0.0941 |
| | | Visit 13 | high | 1 | 7 | [symbol] | (0.006, 0.471) | 31.607 | 0.0101 |
| | | | low | 7 | 1 | [symbol] | [symbol] | 31.607 | 0.0101 |

[symbol]

indicates data missing or illegible when filed

In the OLINK assay, the time points of Interest for EASI 75 response status were Visit 12 LOCF and Visit 14 LOCF. The odds ratios between low group and high group were relatively consistent as the number of responder only change minimally between Visit 12 LOCF and Visit 14 LOCF. (FIG. 102 and FIG. 103). For Flow and NanoString, the time points of interest were visit 7 and visit 13 respectively. The number of responders increased from visit 7 to visit 13 in combining both low and high biomarker groups. Some of the Odds Ratios on the table showed Infinite. This was due to zero responders in the high biomarker group at visit 7. The top biomarkers (FIG. 104-FIG. 106) showed a larger difference at visit 13 between biomarker groups, however, the trend between biomarkers could be observed at visit 7.

### Model Information for Continuous Efficacy Endpoints

Sample size of the biomarker group and treatment arm were evaluated for each biomarker. Table 28. n1-n4 represents the number of patients in each biomarker group and treatment arm combination.

TABLE 28
Sample size for each biomarker

| Biomarker/ARM | GBR | PBO |
|---|---|---|
| High | n1 | n2 |
| Low | n3 | n4 |

If all n′s are greater than or equal to 3., the following model with interaction term was used:

endpoint cfbl˜endpoint bl+ARM+Biomarker+ARM*Biomarker,

where cfbl-change from baseline and bl-baseline.

Otherwise, following model was used within the arm where the sample size of high and low group is greater than or equal to 3,

endpoint cfbl˜endpoint bl+Biomarker

Results of Biomarkers Correlated with EASI Score

Biomarker group effect in GBR 830 arm measured the difference between biomarker high and low group in GBR 830 arm. Top results were selected based on nominal p-value<0.05 at visit 13 (or visit 14 LOCF). The top biomarkers were ranked by the minimum of raw p-value from visit 7 (or visit 12 LOCF) and visit 13 (or visit 14 LOCF). Top five biomarker results showed in Table 29. The missing values from the table were due to small sample size in Placebo arm.

TABLE 29
Top five biomarker results related to EASI change from baseline

| | | | | GBR 830 | | | | Placebo | | | |
|---|---|---|---|---|---|---|---|---|---|---|---|
| | | | | Change from Baseline (%) | | | | Change from Baseline (%) | | | |
| | | | | | | | p-value | | | | p-value |
| | | | | | | Conf. | high vs | | | Conf. | high vs |
| Source | Biomarker | Visit | Group | n | estimatas | Interval | low | n | estimates | Interval | low |
| NanoString | LTB | Visit 7 | high | [symbol] | −52.0 | [symbol] | 0.9999 | 7 | −55.1 | (−77.6, −32.5) | 0.1754 |
| | | | low | 12 | −53.0 | (−70.3, −35.6) | | 3 | −12.5 | (−46.9, 22.0) | |
| | | Visit 13 | high | 7 | −44.4 | (−62.1, −26.7) | 4.0e−04 | [symbol] | — | — | — |
| | | | low | 9 | −83.7 | (−95.7, −71.8) | | 2 | — | — | |
| OLINK | PIK3AP1 | Visit 12 | high | 12 | [symbol] | (−91.5, −65.3) | 0.0017 | 8 | — | — | |
| | | LOCF | low | 13 | −50.2 | (−63.0, −37.4) | | 2 | — | — | |
| | | Visit 14 | high | 12 | −74.8 | (−88.2, −61.3) | 7e−04 | 8 | — | — | |
| | | LOCF | low | 13 | −43.7 | (−56.8, −30.5) | | 2 | — | — | |
| NanoString | SPN | Visit 7 | high | 9 | −50.9 | (−74.8, −27.4) | 0.9992 | [symbol] | −50.0 | (−75.8, −24.2) | 0.7851 |
| | | | low | 11 | −52.9 | [symbol] | | 4 | −30.0 | (−62.7, 2.6) | |
| | | Visit 13 | high | 8 | −48.0 | (−65.5, −30.5) | 0.0014 | 5 | — | — | — |
| | | | low | [symbol] | −85.3 | (−98.7, −71.9) | | 2 | — | — | |
| | MICA | Visit 7 | high | 11 | −54.2 | (−74.5, −34.0) | 0.9982 | [symbol] | −38.4 | (−64.5, −12.3) | [symbol] |
| | | | low | [symbol] | −51.7 | (−73.7, −29.7) | | 4 | −48.7 | (−81.8, −15.6) | |
| | | Visit 13 | high | [symbol] | −85.4 | (−99.1, −71.8) | 0.0017 | 5 | — | — | — |
| | | | low | [symbol] | −54.7 | (−69.2, −40.2) | | 2 | — | — | |
| | CD83 | Visit 7 | high | 8 | [symbol] | (−71.0, −21.2) | [symbol] | 6 | −53.5 | [symbol] | 0.4894 |
| | | | low | 12 | −56.1 | (−73.9, −38.4) | | 4 | −24.9 | (−55.6, 5.8) | |
| | | Visit 13 | high | [symbol] | −43.0 | (−63.3, −22.6) | 0.0017 | [symbol] | — | — | — |
| | | | low | 10 | [symbol] | (−93.0, −69.0) | | 2 | — | — | |

[symbol]

indicates data missing or illegible when filed

For Nanostring, the time points of interest were visit 7 and visit 13 respectively. The top biomarkers (FIG. 107-FIG. 110) showed a larger difference at visit 13 between biomarker groups, however, no difference was observed between high and low group at visit 13. OLINK top biomarker (FIG. 111) showed a consistent difference between the high and low group at visit 12 LOCF and visit 14 LOCF.

Results of Biomarkers Correlated with EASI Subscore

EASI subscore included four scores: Erythema, Excoriation, Induration, and Lichenification. Top results for each subscore were selected based on raw p-value <0.05 biomarker group effect at visit 13 (or visit 14 LOCF) in GBR 830 arm. The top biomarkers were ranked by the minimum of raw p-value from visit 7 (or visit 12LOCF) and visit 13 (or visit 14 LOCF). Top five biomarker results showed Table 30, Table 9.2-5, Table 9.2-7, and Table 9.2-8 for each of the subscores. The missing values from the table were due to small sample size in Placebo arm.

### Erythema Score

TABLE 30
Top five biomarker results related to Erythema score change from baseline

| | | | | GBR830 | | | | Placebo | | | |
|---|---|---|---|---|---|---|---|---|---|---|---|
| | | | | Change from Baseline (%) | | | | Change from Baseline (%) | | | |
| | | | | | | | p-value | | | | p-value |
| | | | | | | Conf. | high vs | | | Conf. | high vs |
| Source | Biomarker | Visit | Group | n | estimates | Interval | low | n | estimates | Interval | low |
| NanoString | YTHDF [symbol] | Visit 7 | high | 12 | [symbol] | (−73.8, −40.0) | 0.0014 | 4 | −32.1 | (−60.0, −4.2) | 0.9873 |
| | | | low | 8 | [symbol] | [symbol] | | [symbol] | −26.0 | (−48.8, −3.2) | |
| | | Visit 13 | high | 9 | −74.2 | [symbol] | 1.1e−04 | 4 | −31.6 | (−53.2, −10.0) | 0.9840 |
| | | | low | 7 | [symbol] | (−42.4, −9.6) | | 3 | −37.7 | (−62.7, −12.7) | |
| Flow | CD4+ | Visit 7 | high | 13 | [symbol] | (−37.8, −1.8) | 0.1117 | 3 | −12.2 | (−49.2, 24.8) | 0.7437 |
| | CCR10+ | | low | 10 | −50.3 | [symbol] | | 5 | −36.2 | (−67.0, −5.5) | |
| | Th22 helper | Vist 13 | high | 9 | −28.3 | (−45.5, −11.1) | 9e−04 | 2 | — | — | — |
| | cells | | low | 11 | −66.6 | (−82.0, −51.3) | | 5 | — | — | |
| NanoString | MAP3K7 | Vist 7 | high | 10 | −58.4 | (−77.2, −39.6) | 0.0064 | 6 | −19.6 | (−42.8, 3.6) | 0.6222 |
| | | | low | 10 | −15.4 | (−33.3, 2.6) | | 4 | −42.3 | (−70.9, −13.6) | |
| | | Visit 13 | high | 7 | −71.0 | (−91.5, −60.6) | 0.0147 | 5 | — | — | — |
| | | | low | 9 | −37.6 | (−55.2, −20.0) | | 2 | — | — | |
| | CD47 | Visit 7 | high | 10 | [symbol] | [symbol] | 0.0302 | 6 | −40.4 | [symbol] | 0.6143 |
| | | | low | 10 | −13.3 | (−34.4, 7.7) | | 4 | −15.4 | (−45.7, 14.9) | |
| | | Visit 13 | high | 8 | −70.1 | [symbol] | 0.0103 | 5 | — | — | — |
| | | | low | 8 | −29.3 | (−51.0, −7.6) | | 2 | — | — | |
| | TLR6 | Visit 7 | high | 12 | −39.8 | (−59.5, −20.2) | 0.9310 | 4 | −18.7 | (−52.7, 15.4) | 0.8563 |
| | | | low | 8 | −29.5 | (−56.0, −3.0) | | 6 | −36.5 | (−64.2, −8.7) | |
| | | Visit 13 | high | 10 | −54.7 | (−82.0, −47.4) | 0.0184 | 2 | — | — | — |
| | | | low | [symbol] | −25.2 | [symbol] | | 5 | — | — | |

[symbol]

indicates data missing or illegible when filed

MAP3K7 and YTHDF2 (FIG. 112 and FIG. 113) showed difference at both visit 7 and visit 13 between biomarker groups in GBR 830 arm. Th22 helper cells (FIG. 114) showed large difference at visit 13 between biomarker groups, and the trend of difference showed up at visit 7 but not significant.

### Excoriation Score

TABLE 31
Top five biomarker results related to Excoriation score change from baseline

| | | | | GBR 830 | | | | Placebo | | | |
|---|---|---|---|---|---|---|---|---|---|---|---|
| | | | | Change from Baseline (%) | | | | Change from Baseline (%) | | | |
| | | | | | | | p-value | | | | p-value |
| | | | | | | Conf. | high vs | | | Conf. | high vs |
| Source | Biomarker | Visit | Group | n | estimates | Interval | low | n | estimates | Interval | low |
| Flow | CD4+ | Visit 7 | high | 13 | −27.8 | (−50.5, −5.1) | 0.5212 | 3 | −13.8 | [symbol] | 0.7371 |
| | CCR10+ | | low | 10 | [symbol] | [symbol] | | 5 | −44.0 | (−79.3, −8.7) | |
| | Th22 helper | Visit 13 | high | [symbol] | −49.3 | (−65.8, −33.0) | 3.1e−04 | 2 | — | — | — |
| | cells | | low | 11 | [symbol] | (−103.2, −73.7) | | 5 | — | — | |
| | CARD11 | Visit 7 | high | 10 | −13.7 | (−30.1, 11.7) | [symbol] | 6 | [symbol] | (−82.9, −24.2) | [symbol] |
| | | | low | 10 | −54.2 | [symbol] | | 3 | 0.7 | (−40.8, 42.3) | |
| | | Visit 13 | high | 7 | −35.4 | (−57.8, −13.0) | 3.1e−04 | 5 | — | — | — |
| | | | low | [symbol] | −88.9 | (−104.5, −73.2) | | 2 | — | — | |
| | CCR5 | Visit 7 | high | 10 | −14.5 | [symbol] | [symbol] | 6 | −44.7 | (−76.2, −13.3) | 0.7546 |
| | | | low | 10 | −54.2 | (−78.2, −30.2) | | 3 | −16.7 | [symbol] | |
| | | Visit 13 | high | 7 | −35.4 | (−57.8, −13.0) | 3.1e−04 | 5 | — | — | — |
| | | | low | [symbol] | −88.9 | (−104.5, −73.2) | | 2 | — | — | |
| NanoString | MAPK11 | Visit 7 | high | 10 | −38.3 | [symbol] | [symbol] | 5 | −27.9 | [symbol] | 0.9418 |
| | | | low | 10 | −36.4 | [symbol] | | 4 | −44.1 | [symbol] | |
| | | Visit 13 | high | 7 | −35.4 | (−57.8, −13.0) | 3.1e−04 | 5 | — | — | — |
| | | | low | [symbol] | [symbol] | (−104.5, −73.2) | | 2 | — | — | |
| | CD180 | Visit 7 | high | 13 | −23.2 | (−49.2, 2.8) | 0.4086 | 3 | −40.0 | (−86.1, 6.1) | 0.0950 |
| | | | low | 7 | −58.5 | (−87.3, −25.8) | | 6 | [symbol] | [symbol] | |
| | | Visit 13 | high | [symbol] | −35.0 | [symbol] | 4.2e−04 | 2 | — | — | — |
| | | | low | [symbol] | [symbol] | (−111.1, −75.7) | | 5 | — | — | |

[symbol]

indicates data missing or illegible when filed

CARD11 (FIG. 115) showed a significant difference at visit 13 between biomarker groups in GBR 830 arm and marginal significant at visit 7. CRRS, CD180, MAPK11, and Th22 helper cells (FIG. 116-FIG. 119) showed a significant difference between biomarker groups in GBR 830 at visit 13, but not visit 7.

Table 32 showed the biomarkers with an early (visit 7) difference between high and low group in GBR 830 arm, and the difference between high and low group increased at visit 13, and improvement could be observed in both biomarker groups in GBR 830 arm (FIG. 120)

TABLE 32
Top biomarkers results related to Excoriation score change from baseline (Visit 7)

| | | | | GBR 830 | | | | Placebo | | | |
|---|---|---|---|---|---|---|---|---|---|---|---|
| | | | | Change from Baseline (%) | | | | Change from Baseline (%) | | | |
| | | | | | | | p-value | | | | p-value |
| | | | | | | Conf. | high vs | | | Conf. | high vs |
| Source | Biomarker | Visit | Group | n | estimates | Interval | low | n | estimates | Interval | low |
| NanoString | CD1B | Visit 7 | high | 9 | −8.6 | (−36.1, 18.9) | 0.0434 | [symbol] | −41.2 | (−71.9, −10.4) | 0.9262 |
| | | | low | 11 | −55.4 | (−77.7, −33.2) | | 3 | −23.9 | (−67.7, 19.9) | |
| | | Visit 13 | high | 7 | −41.6 | (−66.5, −16.8) | 0.0247 | 4 | −52.2 | (−83.2, −21.3) | 0.7720 |
| | | | low | 9 | −87.5 | (−107.1, −67.9) | | 3 | −28.4 | (−63.6, 6.9) | |

[symbol]

indicates data missing or illegible when filed

### Induration Score

TABLE 33
Top five biomarker results related to Induration score change from baseline

| | | | | GBR830 | | | | Placebo | | | |
|---|---|---|---|---|---|---|---|---|---|---|---|
| | | | | Change from Baseline (%) | | | | Change from Baseline (%) | | | |
| | | | | | | | p-value | | | | p-value |
| | | | | | | Conf. | high vs | | | Conf. | high vs |
| Source | Biomarker | Visit | Group | n | estimates | Interval | low | n | estimates | Interval | low |
| NanoString | SPN | Visit 7 | high | 9 | −30.1 | (−52.4, −7.7) | 0.8402 | 6 | [symbol] | (−63.6, −8.1) | 0.7950 |
| | | | low | 11 | −42.7 | (−63.0, −22.4) | | 4 | −15.6 | (−48.9, 17.7) | |
| | | Visit 13 | high | 8 | −32.4 | (−49.8, −15.1) | 6e−04 | 5 | — | — | — |
| | | | low | 8 | −74.0 | (−90.5, −57.5) | | 2 | — | — | |
| OLINK | BACH1 | Visit 12 | high | 11 | −70.4 | (−85.1, −55.8) | 6e−04 | 8 | — | — | |
| | | LOCF | low | 14 | −36.5 | (−49.7, −23.4) | | 2 | — | — | |
| | | Visit 14 | high | 11 | −58.4 | (−75.2, −41.5) | 0.0422 | 8 | — | — | |
| | | LOCF | low | 14 | −35.2 | (−50.3, −20.0) | | 2 | — | — | |
| NanoString | YTHDF2 | Visit 7 | high | 12 | −55.9 | (−71.6, −40.2) | 7e−04 | 4 | −31.1 | (−58.2, −4.0) | 0.9792 |
| | | | low | 8 | −6.8 | (−26.4, 12.8) | | 6 | −24.0 | (−46.6, −1.4) | |
| | | Visit 13 | high | 9 | −70.4 | (−86.5, −54.2) | 0.0071 | 4 | [symbol] | (−60.4, −12.9) | [symbol] |
| | | | low | 7 | −31.0 | (−49.1, −13.0) | | 3 | −35.0 | (−62.9, −7.2) | |
| OLINK | PIK3AP1 | Visit 12 | high | 12 | −69.4 | (−83.8, −55.1) | 7e−04 | 8 | — | — | |
| | | LOCF | low | 13 | −35.9 | (−49.4, −22.4) | | 2 | — | — | |
| | | Visit 14 | high | 12 | −59.6 | (−75.5, −43.7) | 0.0161 | 8 | — | — | |
| | | LOCF | low | 13 | −33.0 | (−48.1, −18.0) | | 2 | — | — | |
| | MMP-1 | Visit 12 | high | 13 | −35.7 | (−52.1, −19.3) | [symbol] | 4 | −28.5 | [symbol] | 0.9410 |
| | | LOCF | low | 12 | −65.7 | (−83.4, −48.0) | | 6 | −39.7 | (−64.1, −15.3) | |
| | | Visit 14 | high | 13 | −26.3 | (−40.0, −12.6) | 0.0011 | 4 | −35.2 | [symbol] | [symbol] |
| | | LOCF | low | 12 | −64.5 | (−79.3, −49.8) | | 6 | −42.8 | (−63.2, −22.5) | |

[symbol]

indicates data missing or illegible when filed

YTHDF2 (FIG. 121), BACH1 (FIG. 123), and PIk3/AP1 (FIG. 124) showed a difference at visit 7 between biomarker groups in GBR 830 arm, and the difference continued at visit 13. MMP-1 (FIG. 125), showed a significant difference between biomarker group in GBR 830 arm at visit 13, but trends observed at visit 7. SPN (FIG. 122) showed a significant difference between biomarker groups in GBR 830 arm at visit 13, but not visit 7.

### Lichenification Score

TABLE 34
Top five biomarker results related to Lichenification score change from baseline

| | | | | GBR830 | | | | Placebo | | | |
|---|---|---|---|---|---|---|---|---|---|---|---|
| | | | | Change from Baseline (%) | | | | Change from Baseline (%) | | | |
| | | | | | | | p-value | | | | p-value |
| | | | | | | Conf. | high vs | | | Conf. | high vs |
| Source | Biomarker | Visit | Group | n | estimates | Interval | low | n | estimates | Interval | low |
| NanoString | LTB | Visit 7 | high | 8 | −14.7 | [symbol] | 0.2222 | 7 | [symbol] | (−59.4, −12.7) | 0.0039 |
| | | | low | 12 | −42.3 | (−60.9, −23.7) | | 3 | [symbol] | (2.2, 74.3) | |
| | | Visit 13 | high | 7 | −17.3 | [symbol] | [symbol] | [symbol] | — | — | — |
| | | | low | 9 | [symbol] | (−78.7, −47.1) | | 2 | — | — | |
| | MICA | Visit 7 | high | 11 | [symbol] | [symbol] | 0.8401 | [symbol] | −19.5 | (−51.4, 12.4) | [symbol] |
| | | | low | [symbol] | [symbol] | (−47.7, 4.0) | | 4 | −5.3 | (−43.8, 33.1) | |
| | | Visit 13 | high | [symbol] | −71.0 | [symbol] | 8e−04 | [symbol] | — | — | — |
| | | | low | [symbol] | −21.5 | [symbol] | | 2 | — | — | |
| | CD4 | Visit 7 | high | 10 | −23.3 | (−48.4, 1.9) | 0.8741 | 5 | −12.1 | [symbol] | [symbol] |
| | | | low | 10 | [symbol] | [symbol] | | 5 | −16.3 | [symbol] | |
| | | Visit 13 | high | [symbol] | −23.1 | (−41.4, −4.8) | 0.0017 | 2 | — | — | — |
| | | | low | 7 | [symbol] | (−86.0, −47.1) | | 5 | — | — | |
| | CD83 | Visit 7 | high | [symbol] | [symbol] | (−44.7, 7.0) | [symbol] | 6 | −35.2 | [symbol] | 0.1016 |
| | | | low | 12 | −38.2 | [symbol] | | 4 | 17.7 | [symbol] | |
| | | Visit 13 | high | [symbol] | −14.3 | [symbol] | 0.0022 | 5 | — | — | — |
| | | | low | 10 | −58.8 | (−75.0, −42.2) | | 2 | — | — | |
| | ATG7 | Visit 7 | high | 10 | −40.8 | (−61.5, −20.2) | 0.6776 | 7 | 7.9 | [symbol] | 0.0183 |
| | | | low | 10 | −21.2 | [symbol] | | 3 | [symbol] | (−103.4, −25.5) | |
| | | Visit 13 | high | [symbol] | −61.1 | (−78.9, −43.2) | 0.0034 | 5 | — | — | — |
| | | | low | 7 | −15.7 | (−38.7, 7.3) | | 2 | — | — | |

[symbol]

indicates data missing or illegible when filed

CD4, CD83, LTB, MICA, and ATG7 (FIG. 126-FIG. 130) showed significant difference between biomarker groups in GBR 830 arm at visit 13, but not at visit 7.

Additional erythema, induration/papules, excoriation and lichenification results are presented in FIG. 131.

### Discussion

Biomarker analysis of GBR830-201 was focused on GBR 830 target engagement, pharmacodynamics modulation of markers implicated in the pathogenies of Atopic dermatitis and of biomarker for pathways that are currently under therapeutic evaluation in Atopic dermatitis.

The expression of OX40, the pharmacologic target of GBR 830, was found to be reduced at visit 7 and 13. At visit 13, the expression change from baseline became nominally significant.

Reduction in TRAF2, TBK1, TANK, integral parts of Ox40/TNF-R pathway, was also found to be consistent with target engagement and provide evidence for the functional blockade of OX40. Furthermore a potential surrogate marker was identified as IFI27 gene expression sharply correlated with OX40 expression. In aggregate GBR830 reduced the expression of its pharmacologic target Ox40 and reduced the expression of several components of the Ox40/TNFR signaling pathway providing evidence for target engagement and functional modulation of OX40 by GBR 830.

While the acute phase of Atopic dermatitis is believed to be driven by Th2 cytokines, Th1 mediators are upregulated in the chronic phase of AD. GBR830 showed trends to suppress Th1 T cell derived cytokines such as IFNG and Th1 pathway biomarker CXCL9, CXCL10.

For Th2 pathway GBR 830 showed no trends to reduce in IL4. CCL11 showed trends to reduce at visit 13 on mRNA but not protein level. CCL17 showed trends to reduce at visit 13 for both NanoString and RT-PCR, however, same trends observed in the Placebo arm too.

KI67 which is found elevated in the skin of Atopic dermatitis patients due to the elevated proliferation of keratinocytes was found to be downregulated by GBR830, likely consistent with reduce epidermal proliferation and reduced epidermal thickening upon GBR830 treatment.

Altogether data point at a reduction of biomarkers implicated in the chronic phase of atopic dermatitis by GBR830

Biomarker for other pathways that are currently under evaluation for the treatment of Atopic dermatitis (e.g. IL31R, IL33R, TSLPR) did not show modulation by GBR 830 which potentially differentiates GBR830 from other currently tested exploratory treatment modalities.

Other biomarker that were found downregulated by GBR830 include BLNK, a B cell adapter protein and SMAD2, a component of TGFB signaling pathway. Interestingly TGFB/SMAD2 are recognized drivers of fibrotic processes and fibrosis is also a recognized pathomechanistic pillar in chronic Atopic dermatitis.

### Additional Data

### OX40 and Ox40 Pathway Components TRAF2, TANK and TBK1 Reduced by GBR830

The inventors have shown (FIG. 132-FIG. 134) that a reduction of OX40 and Ox40 pathway components TRAF2 and TANK is induced by GBR830, consistent with target engagement and impaired OX40 signaling. The suppression of TBK1 (TANK Binding Kinase 1) linked to augmented T reg numbers and function, this is prima facie evidence for target engagement and emerging Ox40 response signature.

### GBR830 Impacts on Multiple Teff Cytokines

The inventor have shown Reduction of multiple Th1, Th2, Th17 related cytokines and chemokines is induced by GBRB30 (FIG. 135).

Strong evidence particularly for IFN signaling and pathway components.

### GBR830 Dependent Decrease in OX40 in Epidermis and Dermis

Independent analysis (FIG. 136) confirms reduced expression of OX40 in the dermis and extends finding to epidermis.

Evidence for target engagement by GBR830 in both skin compartments.

### IFI27 (Interferon Alpha Inducible Protein 27) Correlates With OX40 Expression Across All Visits

IFI27 displays tight correlation with Ox40 expression at baseline and under GBR830 treatment (FIG. 137). Potential new insights into OX40 mechanism and link to Type I interferon pathway. IFI27 is Induced by Type I and II interferons and has a broad expression pattern in human tissues although its function is currently unknown. Next steps: Link the identified correlation/info with the ongoing post-doctoral work within SLE.

### Summary (I)

It has been shown that there is a direct effect of GBR830 on pharmacologic target OX40/TNFRSF14. Three proximal components of canonic TNFRS signaling impacted by GBR830:
TRAF2 (mRNA, no effect on protein);
TANK (mRNA, protein not determined);
TBK1 (mRNA, protein not determined).

In addition IFI27 shows good correlation with OX40 expression across all visits.

Target engagement of GBR830 has an impact on OX40 signaling machinery.

TRAF2, TANK, TBK1 are potential new biomarker to determine PD in AD and other diseases, Further validation required.

IFI27 biology and link to Ox40, AD unknown.

### Th1 Biomarker Confirmed and Extended

IFNγ key Th1 cytokine are upregulated in chronic AD (FIG. 138). IFNγ signature suppressed by G8R830 represented by key chemokines i.e., CXCI9, 10 (FIG. 139).

Next steps will include an Investigation in the functional impact, such as a potential reduction of the receptor involved in CXCL9, 10, 11 signaling i.e., CXCR3. Further work will be undertaken to link the identified signature/info with within SLE.

### GBR830 Responder Display Reduced IFNγ Signature

GBR830 responder (solid red and green bars FIG. 140) at both, V7 and V13 display reduced CXCL9, 10, 11 expression. Correlative finding; supports PD effect of OX40 on IFNG/Th1 pathway.

### B Cell Adapter Protein Downregulated by GBR830 Treatment

GBR830 induces a reduction in B Cell Linker BINK Figure (FIG. 141). BLNK encodes a cytoplasmic linker or adaptor protein that plays a critical role in B cell development.

### Reduction of Smad2 by GBR830

GBR830 induces a roduction In SMAD Family Member 2 (Smad2) (FIG. 142). Smad2 signaling has been linked to inflammation and the development of chronic fibrogenesis.

Reduction of Ki67 in the epidermis by GBR830

GBR830 induces a reduction of Ki67 in the epidermis (FIG. 143).

### Example 4: Dosage Regimen

### Dosage Regimen

The dose regimen for this study has been determined considering all available safety and PK data from clinical experience with GBR 830 and nonclinical studies.

Pharmacokinetic and efficacy data in subjects with moderate-to-severe AD (Study GBR 830 201), PK and safety data after SC injection and IV infusion to healthy subjects (Study GBR 830-102), and PK and safety data after IV infusion up to 10 mg/kg (Study GBR 830-101), available safety data from the ongoing phase 1 study (Study GBR 830-103), and the receptor occupancy data of GBR 830 in activated human whole blood were considered in determination of the dosage regimen for the current GBR 830-204 study.

Based on the available results, GBR 830 is safe and well tolerated up to 40 mg/kg dose level and showed dose proportional PK across the evaluated dose range (0.3 mg/kg to 40 mg/kg). The absolute bioavailability of GBR 830 after SC injection is approximately 65%. The average ty of GBR 830 ranged from 10 to 15 days, and appeared to be independent of dose level or route of administration. Receptor occupancy experiment with GBR 830 in activated human whole blood indicated that maximum receptor occupancy (ROₘₐₓ) was achieved at a concentration of approximately 25 μg/ml of GBR 830 and a 50% receptor occupancy (RO₅₀) was achieved at a concentration of around 3 μg/mL of GBR 830. In study GBR 830-201, the average C_{trough} was maintained at around 30 μg/ml over the entire dosing interval, similar to the concentration required for ROₘₐₓ.

The dosing schedule for the current study includes a loading dose followed by maintenance dosing for the GBR 830 treatment arms (Groups 1, 2 and 3). The loading dose for each group is selected based on the corresponding maintenance dose and the dosing frequency in order to achieve steady state levels faster. The same regimen is followed for the placebo arm (Group 4) to maintain the blind.
The highest GBR 830 dosage regimen (Group 1) includes a 600 mg GBR 830 SC loading dose followed by 300 mg GBR 830 SC q2w maintenance dosing. At this dose level, the average steady state C_{trough} is anticipated to be approximately 46 μg/mL, which is slightly higher than what was achieved in the GBR 830-201 study. With this dosage regimen, the average steady state C_{trough} values is maintained above the concentration that elicited ROₘₐₓ in the in vitro experiments and therefore has been selected as the highest dose for the current study.
The middle GBR 830 dosage regimen (Group 2) includes a 600 mg GBR 830 SC loading dose followed by 300 mg GBR 830 SC q4w maintenance dosing. At this dose level, the average steady state C_{trough} is anticipated to be approximately 16 μg/mL, which is less than what is required for ROₘₐₓ, yet greater than the level that elicited RO₅₀ in the in vitro experiments and therefore has been selected as the middle dose.
The lowest GBR 830 dosage regimen (Group 3) includes a 150 mg GBR 830 SC loading dose followed by 75 mg GBR 830 SC q4w maintenance dosing. The average C_{trough} anticipated with this regimen is approximately 4.0 μg/ml, similar to the concentration required to elicit RO₅₀, in in vitro experiments.

With the above GBR 830 dosage regimens, an approximate 10-fold spread in the average steady state C_{trough}, and approximate 8-fold spread in average steady state AUC4week are expected and are considered adequate to meet the objectives of the study.

### Study Drug Materials and Management

A description of treatment groups and their respective dose regimens is provided in Table 35. All subjects receive a loading dose consisting of 2 SC injections, and maintenance dosing consisting of 1 SC injection per dose, to maintain the blind.

TABLE 35
Treatment Groups and Dose Regimens

| Treatment | | | Maintenance Dose Schedule |
|---|---|---|---|
| Group | Loading Dose (Day 1) | Maintenance Dose | (Starting on Week 2) |
| Group 1 | 2 GBR 830 SC injections | 1 GBR 830 SC injection | GBR 830: Weeks 2, 4, |
| | (2 × 2 mL of 150 mg/mL | (1 × 2 mL of 150 mg/mL | 6, 8, 10, 12, and 14 |
| | formulation) | formulation) | |
| Group 2 | 2 GBR 830 SC injections | 1 GBR 830 SC injection | GBR 830: Weeks 4, 8 |
| | (2 × 2 mL of 150 mg/mL | (1 × 2 mL of 150 mg/mL | and 12 |
| | formulation) | 1 placebo SC injection | Placebo: Weeks 2, 6, 10 |
| | | (1 × 2 mL) | and 14 |
| Group 3 | 2 GBR 830 SC injections | 1 GBR 830 SC injection | GBR 830; Weeks 4, 8 |
| | (2 × 2 mL of 37.5 mg/mL | (1 × 2 mL of 37.5 | and 12 |
| | formulation) | 1 placebo SC injection | Placebo: Weeks 2, 6, 10 |
| | | (1 × 2 mL) | and 14 |
| Group 4 | 2 placebo SC injections | 1 placebo SC injection | Placebo: Week 2, 4, 6, |
| | (2 × 2 mL) | (1 × 2 mL) | 8, 10, 12, and 14 |

### Investigational Product

GBR 830 is provided as lyophilized powder in a 10 mL glass vial. Each vial contains 192 mg of GBR 830, 160 mg of sucrose, 3.1 mg of histidine, and 0.4 mg of polysorbate 80, and is designed to deliver 150 mg of GBR 830 in 1.0 ml injection after reconstitution with 1.1 ml of sterile water for injection.

### Placebo

Placebo is supplied as 200 mg of sucrose, 4 mg of histidine, and 0.5 mg of polysorbate 80. Each vial of GBR 830 placebo is designed to be reconstituted with 1.3 mL of sterile water for injection to yield corresponding placebo.

1. A method of treating or preventing an OX40-mediated disorder comprising administering to a subject in need thereof an anti-OX40 antagonist antibody, wherein said antagonist antibody induces modulation of Th1 and/or Th2 and/or Th17/Th22 markers.

2. The method of claim 1, wherein the Th1 markers which are modulated are selected from the group consisting of IFNγ and CXCL10; the Th2 markers which are modulated are selected from the group consisting of IL-31, CCL11, CCL17, and TSLPR; and said Th17/Th22 markers which are modulated are selected from the group consisting of IL-23p19, IL-8, and S100As.

3. The method of claim 2, wherein the Th1 and/or Th2 and/or Th17/Th22 markers are downregulated.

4. The method of claim 1, wherein the OX40-mediated disorder is atopic dermatitis.

5. The method of claim 1, wherein the OX40-mediated disorder is moderate-to-severe atopic dermatitis.

6. The method of claim 1, wherein said antibody is administered intravenously at two doses of about 10 mg/Kg of the subject's body weight, around four weeks apart.

7. The method of anyone of claim 1, wherein the OX40-mediated disorder is selected from the group consisting of rheumatoid arthritis, autoimmune uveitis, multiple sclerosis, lupus, graft-versus-host disease (GVHD), scleroderma, hidradenitis, and ulcerative colitis.
